(19) United States
(12) Patent Application Publication
(10) Pub. No.: US 2021/0186954 A1
(43) Pub. Date: Jun. 24, 2021

(54) DRUG PRODUCTS FOR NASAL ADMINISTRATION AND USES THEREOF

(71) Applicant: Summit Biosciences Inc., Lexington, KY (US)

(72) Inventors: Gregory G. Plucinski, Nicholasville, KY (US); Kristi R. Sims, Versailles, KY (US); Bryan S. Abney, Richmond, KY (US); Meng Zhong, Westerville, OH (US); Myron Vance, Lexington, KY (US); Adrian T. Raiche, Lexington, KY (US)

(21) Appl. No.: 17/168,519

(22) Filed: Feb. 5, 2021

Related U.S. Application Data
(63) Continuation of application No. PCT/US2019/045300, filed on Aug. 6, 2019.
(60) Provisional application No. 62/743,401, filed on Oct. 9, 2018.
(60) Provisional application No. 62/715,080, filed on Aug. 6, 2018.

(51) Int. Cl. A61K 31/485; A61K 47/10; A61K 47/12; A61K 9/08; A61K 47/18; A61K 9/00

(52) U.S. Cl. CPC A61K 31/485; A61K 47/10; A61K 9/0043; A61K 9/08; A61K 47/183; A61K 47/12

(57) ABSTRACT

Provided herein are drug products adapted for nasal delivery comprising a device and a pharmaceutical composition comprising an opioid receptor antagonist, pharmaceutical compositions comprising an opioid receptor antagonist, and methods of use thereof.

### CROSS-REFERENCE TO RELATED APPLICATIONS

This application is a continuation of International Application Number PCT/US2019/45300 filed Aug. 6, 2019, which claims priority to U.S. Ser. No. 62/715,080 filed Aug. 6, 2018 and U.S. Ser. No. 62/743,401 filed Oct. 9, 2018, the contents of each of which are incorporated herein by reference.

### BACKGROUND OF THE INVENTION

Opioid receptors are G protein-coupled receptors (GPCRs) that are activated both by endogenous opioid peptides and by clinically important alkaloid analgesic drugs such as morphine. There are three principal types of opioid receptors: the δ-opioid receptor, the κ-opioid receptor, and the μ-opioid receptor. Opioids depress respiration, which is controlled principally through medullary respiratory centers with peripheral input from chemoreceptors and other sources. Opioids produce inhibition at the chemoreceptors via μ-opioid receptors and in the medulla via μ- and δ-opioid receptors. While there are a number of neurotransmitters mediating the control of respiration, glutamate and γ-aminobutyric acid (GABA) are the major excitatory and inhibitory neurotransmitters, respectively. This explains the potential for interaction of opioids with benzodiazepines and alcohol: both benzodiazepines and alcohol facilitate the inhibitory effect of GABA at the GABA_{A} receptor, while alcohol also decreases the excitatory effect of glutamate at NMDA receptors. Oxycodone and other opioid painkillers, as well as heroin and methadone are all implicated in fatal overdose. Heroin has three metabolites with opioid activity. Variation in the formation of these metabolites due to genetic factors and the use of other drugs could explain differential sensitivity to overdose. Metabolites of methadone contribute little to its action. However, variation in rate of metabolism due to genetic factors and other drugs used can modify methadone concentration and hence overdose risk. The degree of tolerance also determines risk. Tolerance to respiratory depression is less than complete, and may be slower than tolerance to euphoric and other effects. One consequence of this may be a relatively high risk of overdose among experienced opioid users. While agonist administration modifies receptor function, changes (usually in the opposite direction) also result from use of antagonists, for example, supersensitivity to opioids following a period of administration of antagonists such as naltrexone.

In the United States, mortality rates closely correlate with opioid sales. In 2008, approximately 36,450 people died from drug overdoses. At least 14,800 of these deaths involved prescription opioid analgesics. Moreover, according to the Substance Abuse and Mental Health Services Administration, the number/rate of Americans 12 years of age and older who currently abuse pain relievers has increased by 20 percent between 2002 and 2009. In New York City, between 1990 and 2006, the fatality rate from prescription opioids increased seven-fold, from 0.39 per 100,000 persons to 2.7. Drugs classed as prescription opioids in this study include both typical analgesics, such as OXYCONTIN® (oxycodone HCl controlled-release) and methadone (used in the treatment of dependence on other opioids such as heroin and also prescribed for pain), but the increase in the rate of drug overdose over the 16 years of the study was driven entirely by overdoses of typical analgesics. Over the same time period, methadone overdoses remained stable, and overdoses from heroin declined. Whites were more likely than blacks and Latinos to overdose on these analgesics, and deaths mostly occurred in neighborhoods with lower rates of poverty, suggesting differential access to doctors who can write painkiller prescriptions may be a driving force behind the racial disparity. (Cerda et al. “Prescription opioid mortality trends in New York City, 1990-2006: Examining the emergence of an epidemic,” Drug and Alcohol Dependence Volume 132, Issues 1-2, 1 Sep. 2013, 53-62.)

Naloxone is an opioid receptor antagonist that is approved for use by injection for the reversal of opioid overdose and for adjunct use in the treatment of septic shock. It is currently being used mainly in emergency departments and in ambulances by trained medical professionals. There have been efforts to expand its use by providing the drug to some patients with take-home opioid prescriptions and those who inject illicit drugs, potentially facilitating earlier administration of the drug. The UN Commission on Narcotics Drugs “encourages all Member States to include effective elements for the prevention and treatment of drug overdose, in particular opioid overdose, in national drug policies, where appropriate, and to share best practices and information on the prevention and treatment of drug overdose, in particular opioid overdose, including the use of opioid receptor antagonists such as naloxone.”

U.S. Pat. No. 4,464,378 describes a method for eliciting an analgesic or narcotic antagonist response in a warm-blooded animal, which comprises administering intranasally (IN) to said animal to elicit a narcotic antagonist response, a narcotic antagonist effective amount of naloxone. WO 82/03768 discloses a composition that contains 1 mg of naloxone hydrochloride per 0.1 ml of solution adapted for nasal administration used in the treatment of narcotic induced respiratory depression (overdose) at a dosage approximately the same as that employed for intravenous (IV), intramuscular (IM) or subcutaneous (SQ) administration. WO 00/62757 teaches pharmaceutical compositions for IN or oral (PO) administration which comprise an opioid antagonist, such as naloxone for application by spray in the reversal of opioid depression for treatment of patients suffering from opioid over-dosage, wherein the spray applicator is capable of delivering single or multiple doses and suitable dosage units are in the range of 0.2 to 5 mg.

The use of nasal naloxone is not without controversy. For instance, Loimer et al. (International Journal of Addictions, 29(6), 819-827, 1994) reported that the nasal administration of naloxone is as effective as the intravenous route in opiate addicts, however, Dowling et al. (Ther Drug Monit, Vol 30, No 4, August 2008) reported that naloxone administered intranasally displays a relative bioavailability of 4% only and concluded that the IN absorption is rapid but does not maintain measurable concentrations for more than an hour.

One early study of 196 consecutive patients with suspected opioid overdose conducted in an urban out-of-hospital setting, had shown the mean interval from emergency medical services (EMS) arrival to a respiratory rate of ≥10 breaths/min was 9.3±4.2 min with administration of naloxone 0.4 mg IV, versus 9.6±4.58 min with administration of naloxone 0.8 mg SQ. The authors concluded that the slower rate of absorption via the SQ route was offset by the delay in establishing an IV line. (Wanger et al., Intravenous vs subcutaneous naloxone for out-of-hospital management of presumed opioid overdose. Acad Emerg Med. 1998 April; 5(4):293-9).

The Denver Health Paramedic system subsequently investigated the efficacy and safety of atomized intranasal naloxone for the treatment of suspected opiate overdose (Barton, et al., Efficacy of intranasal naloxone as a needleless alternative for treatment of opioid overdose in the prehospital setting. J Emerg Med, 2005. 29(3): p. 265-71). All adult patients encountered in the prehospital setting as suspected opiate overdose, found down, or with altered mental status who met the criteria for naloxone administration were included in the study. IN naloxone (2 mg) was administered immediately upon patient contact and before IV insertion and administration of IV naloxone (2 mg). Patients were then treated by EMS protocol. The main outcome measures were: time of IN naloxone administration, time of IV naloxone administration, time of appropriate patient response as reported by paramedics. Ninety-five patients received IN naloxone and were included in the study. A total of 52 patients responded to naloxone by either IN or IV, with 43 (83%) responding to IN naloxone alone. Seven patients (16%) in this group required further doses of IV naloxone. The median times from arrival at patient side to awakening and from administration of the IN naloxone to patient awakening were 8.0 minutes and 3.0 minutes respectively.

The Drug Overdose Prevention and Education (DOPE) Project was the first naloxone prescription program (NPP) established in partnership with a county health department (San Francisco Department of Public Health), and is one of the longest running NPPs in the USA. From September 2003 to December 2009, 1,942 individuals were trained and prescribed naloxone through the DOPE Project, of whom 24% returned to receive a naloxone refill, and 11% reported using naloxone during an overdose event. Of 399 overdose events where naloxone was used, participants reported that 89% were reversed. In addition, 83% of participants who reported overdose reversal attributed the reversal to their administration of naloxone, and fewer than 1% reported serious adverse effects. Findings from the DOPE Project add to a growing body of research that suggests that intravenous drug users (IDUs) at high risk of witnessing overdose events are willing to be trained on overdose response strategies and use take-home naloxone during overdose events to prevent deaths (Enteen, et al., Overdose prevention and naloxone prescription for opioid users in San Francisco. J Urban Health. 2010 December; 87(6):931-41).

Another reported study reviewed EMS and hospital records before and after implementation of a protocol for administration of intranasal naloxone by the Central California EMS Agency in order to compare the prehospital time intervals from patient contact and medication administration to clinical response for IN versus intravenous IV naloxone in patients with suspected narcotic overdose. The protocol for the treatment of opioid overdose with intranasal naloxone was as follows: “Intranasal (IN)-Administer 2 mg intranasally (1 mg per nostril) using mucosal atomizer device (MAD™) if suspected narcotic intoxication and respiratory depression (rate 8 or less). This dose may be repeated in 5 minutes if respiratory depression persists. Respirations should be supported with a bag valve mask until respiratory rate is greater than 8. Intramuscular (IM)—Administer 1 mg if unable to administer intranasally (see special considerations). May repeat once in 5 minutes. Intravenous (IV)—Administer 1 mg slow IV push if no response to intranasal or IM administration after 10 minutes. Pediatric dose—0.1 mg/kg intranasally, if less than 10 kg and less than 1 year old”. Patients with suspected narcotic overdose treated in the prehospital setting over 17 months, between March 2003 and July 2004 were included. Paramedics documented dose, route of administration, and positive response times using an electronic record. Clinical response was defined as an increase in respiratory rate (breaths/min) or Glasgow Coma Scale score of at least 6. Main outcome variables included time from medication to clinical response and time from patient contact to clinical response. Secondary variables included numbers of doses administered and rescue doses given by an alternate route. Between-group comparisons were accomplished using t-tests and chi-square tests as appropriate. One hundred fifty-four patients met the inclusion criteria, including 104 treated with IV and 50 treated with IN naloxone. Clinical response was noted in 33 (66%) and 58 (56%) of the IN and IV groups, respectively (p=0.3). The mean time between naloxone administration and clinical response was longer for the IN group (12.9 vs. 8.1 min, p=0.02). However, the mean times from patient contact to clinical response were not significantly different between the IN and IV groups (20.3 vs. 20.7 min, p=0.9). More patients in the IN group received two doses of naloxone (34% vs. 18%, p=0.05), and three patients in the IN group received a subsequent dose of IV or IM naloxone. (Robertson et al., Intranasal naloxone is a viable alternative to intravenous naloxone for prehospital narcotic overdose. Prehosp Emerg Care. 2009 October-December; 13(4):512-5).

In August 2006, the Boston Public Health Commission passed a public health regulation that authorized an opioid overdose prevention program that included intranasal naloxone education and distribution of the spray to potential bystanders. Participants were instructed by trained staff to deliver 1 mL (1 mg) to each nostril of the overdose victim. After 15 months, the program had provided training and intranasal naloxone to 385 participants who reported 74 successful overdose reversals (Doe-Simkins et al. Overdose prevention education with distribution of intranasal naloxone is a feasible public health intervention to address opioid overdose. Am J Public Health. 2009; 99:788-791).

Overdose education and nasal naloxone distribution (OEND) programs are community-based interventions that educate people at risk for overdose and potential bystanders on how to prevent, recognize and respond to an overdose. They also equip these individuals with a naloxone rescue kit. To evaluate the impact of OEND programs on rates of opioid related death from overdose and acute care utilization in Massachusetts, an interrupted time series analysis of opioid related overdose death and acute care utilization rates from 2002 to 2009 was performed comparing community-year strata with high and low rates of OEND implementation to those with no implementation. The setting was nineteen Massachusetts communities (geographically distinct cities and towns) with at least five fatal opioid overdoses in each of the years 2004 to 2006. OEND was implemented among opioid users at risk for overdose, social service agency staff, family, and friends of opioid users. OEND programs equipped people at risk for overdose and bystanders with nasal naloxone rescue kits and trained them how to prevent, recognize, and respond to an overdose by engaging emergency medical services, providing rescue breathing, and delivering naloxone. Among these communities, OEND programs trained 2,912 potential bystanders who reported 327 rescues. Both community-year strata with 1-100 enrollments per 100,000 population (adjusted rate ratio 0.73, 95% confidence interval 0.57 to 0.91) and community-year strata with greater than 100 enrollments per 100,000 population (0.54, 0.39 to 0.76) had significantly reduced adjusted rate ratios compared with communities with no implementation. Differences in rates of acute care hospital utilization were not significant. Opioid overdose death rates were reduced in communities where OEND was implemented. This study provides observational evidence that by training potential bystanders to prevent, recognize, and respond to opioid overdoses, OEND is an effective intervention (Walley et al., Opioid overdose rates and implementation of overdose education and nasal naloxone distribution in Massachusetts: interrupted time series analysis. BMJ 2013; 346:f174).

Naloxone prescription programs are also offered by community-based organizations in Los Angeles and Philadelphia. Programs in both cities target IDUs. Studies which recruited 150 IDUs across both sites for in-depth qualitative interviews compared two groups of IDUs, those who had received naloxone prescriptions and those who had never received naloxone prescriptions. In both L.A. and Philadelphia, IDUs reported successfully administering naloxone to reverse recently witnessed overdoses. Reversals often occurred in public places by both housed and homeless IDUs. Despite these successes, IDUs frequently did not have naloxone with them when they witnessed an overdose. Two typical reasons reported were naloxone was confiscated by police, and IDUs did not feel comfortable carrying naloxone in the event of being stopped by police. Similarly, some untrained IDUs reported discomfort with the idea of carrying naloxone on them as their reason for not gaining a prescription.

A randomized trial comparing 2 mg naloxone delivered intranasally with a mucosal atomizer to 2 mg intramuscular naloxone was reported by Kelly et al., in 2005 (Med J Aust. 2005 Jan. 3; 182(1):24-7). The study involved 155 patients (71 IM and 84 IN) requiring treatment for suspected opiate overdose and attended by paramedics of the Metropolitan Ambulance Service (MAS) and Rural Ambulance Victoria in Victoria, Australia. The IM group had more rapid response than the IN group, and were more likely to have more than 10 spontaneous respirations per minute within 8 minutes (82% v. 63%; P=0.0173). There was no statistically significant difference between the IM and IN groups for needing rescue naloxone (13% [IM group] v. 26% [IN group]; P=0.0558). The authors concluded that IN naloxone is effective in treating opiate-induced respiratory depression, but is not as effective as IM naloxone.

Kerr et al. (Addiction. 2009 December; 104(12):2067-74) disclosed treatment of heroin overdose by intranasal administration of naloxone constituted in a vial as a preparation of 2 mg in 1 mL. Participants received 1 mg (0.5 ml) in each nostril. The rate of response within 10 minutes was 60/83 (72.3%) for 2 mg IN naloxone versus 69/89 (77.5%) for 2 mg IM naloxone. The mean response times were 8.0 minutes and 7.9 minutes for IN and IV naloxone respectively. Supplementary naloxone was administered to fewer patients who received IM naloxone (4.5%) than IN (18.1%).

WO2012156317 describes a study in which naloxone, 8 mg and 16 mg, was administered as 400 μL IN (200 μL per nostril). The administration was performed as follows: The pump of the nasal spray was primed by removing the cap and pressing downward. This is repeated at least 6 times or until a fine spray appears; priming is done just prior to dosing. The subject is in a standing or upright position and should gently blow the nose to clear the nostrils. The subject should tilt the head forward slightly and gently close one nostril by pressing the outside of the nose with a finger on the nostril to be closed. The device is inserted into the open nostril and it is sprayed 2 times into the nostril. The subject should gently breath inward through the nostril, the device is removed, and the steps are repeated for the other nostril. The mean Tₘₐₓ values were reported to be 0.34 h (20.4 min) and 0.39 h (23.4 min) for the 8 and 16 mg doses respectively.

Wermeling (Drug Deliv Transl Res. 2013 Feb. 1; 3(1): 63-74) teaches that the initial adult dose of naloxone in known or suspected narcotic overdose is 0.4 to 2 mg, which may be repeated to a total dose of 10 mg and that the current formulations of naloxone are approved for intravenous (IV), intramuscular (IM) and subcutaneous (SC) administration, with IV being the recommended route. Wermeling also predicts that a 2 mg nasal solution dose of naloxone will likely have a Cₘₐₓ of 3-5 ng/mL and a tₘₐₓ of approximately 20 minutes.

Since the onset of action of naloxone used in opioid overdose cases should be as fast as possible, naloxone is thus far mainly administered intravenously or intramuscularly by emergency health care personnel. Due to a high first pass metabolism, oral dosage forms comprising naloxone display a low bioavailability and thus seem to be not suitable for such purposes. The administration of naloxone via injection into the blood stream or into the muscle requires first of all trained medical personnel (for intravenous injection) or a trained care-person (for intramuscular injection). Secondly, depending on the constitution of the addict and the period of intravenous drug abuse, it can be particularly difficult to find access into a vein of the addict's body for administering naloxone intravenously. Clearly, there is a risk of exposure to blood borne pathogens for the medical personnel or the trained carer since a large population of drug addicts suffers from blood borne pathogen induced diseases such as HIV, hepatitis B and C, and the like since accidental needlestick is a serious safety concern. 385,000 needle-stick injuries have been estimated to have occurred in the year 2000 in the US alone (Wilburn, Needlestick and sharps injury prevention, Online J Issues Nurs 2004 Sep. 30; 9(3):5).

Naloxone has a relatively short half-life of compared to some longer-acting opioid formulations and so after a typical therapeutic dose of naloxone is administered to an opioid overdose patient there is often the need to re-administer naloxone, in some cases even several times, and it is important to seek immediate medical attention.

Furthermore, it has been suggested that in view of the growing opioid overdose crisis in the US, naloxone should be made available over-the-counter (OTC), which would require a device, such as a nasal spray device, that untrained consumers are able to use safely. A nasal spray device that was pre-filled with a naloxone formulation would also be less likely to be confiscated by police than the system developed by some EMS programs that combines an FDA-approved naloxone injection product with a marketed, medical device called the Mucosal Atomization Device.

Thus, there remains a need for durable, easy-to-use, needleless devices with storage-stable formulations, that can enable untrained individuals to quickly deliver a therapeutically effective dose of a rapid-acting opioid antagonist to an opioid overdose patient. The therapeutically effective dose should be sufficient to obviate the need for the untrained individual to administer either a second dose of opioid antagonist or an alternative medical intervention to the patient, and to stabilize the patient until professional medical care becomes available. The devices described herein meet this and other needs.

### SUMMARY

Provided are devices adapted for nasal delivery of a pharmaceutical composition to a patient, comprising a therapeutically effective amount of an opioid antagonist selected from naloxone and pharmaceutically acceptable salts and hydrates thereof and chlorobutanol, and wherein the therapeutically effective amount of the opioid antagonist is equivalent to about 2 mg to about 12 mg of naloxone hydrochloride.

Also provided are methods of treating opioid overdose or a symptom thereof, comprising nasally administering to a patient in need thereof a therapeutically effective amount of an opioid antagonist selected from naloxone and pharmaceutically acceptable salts and hydrates thereof, wherein the therapeutically effective amount is equivalent to about 2 mg to about 12 mg of naloxone hydrochloride.

In one aspect, described herein is a pharmaceutical composition comprising naloxone hydrochloride or a hydrate thereof. In some embodiments, the pharmaceutical composition comprises a hydrate of naloxone hydrochloride, e.g., naloxone hydrochloride dihydrate.

In some embodiments, the pharmaceutical composition is an aqueous solution. In some embodiments, the pharmaceutical composition comprises chlorobutanol. In some embodiments, the pharmaceutical composition comprises a polyol less than 300 Da, e.g., propylene glycol or glycerin. In some embodiments, the pharmaceutical composition comprises glycerin. In some embodiments, the pharmaceutical composition comprises an acid. In some embodiments, the acid is citric acid. In some embodiments, the pharmaceutical composition comprises a buffer. In some embodiments, the buffer comprises, e.g., consists essentially of or consists of, citric acid and trisodium citrate dihydrate. In some embodiments, the buffer is an acetate buffer. In some embodiments, the pharmaceutical composition comprises an isotonicity agent. In some embodiments, the isotonicity agent is sodium chloride. In some embodiments, the composition comprises a stabilizing agent. In some embodiments, the stabilizing agent is selected from the group consisting of EDTA and disodium ETDA.

In some embodiments, the volume of the aqueous solution is from about 50 μL to about 200 μL. In some embodiments, the volume of the aqueous solution is from about 80 μL to about 150 μL. In some embodiments, the volume of the aqueous solution is from about 90 μL to about 120 μL. In some embodiments, the volume of the aqueous solution is about 100 μL of the aqueous solution. In some embodiments, the volume of the aqueous solution is 133 μL of the aqueous solution.

In some embodiments, the concentration of naloxone hydrochloride or hydrate thereof is greater than 16 mg per 100 μL of the aqueous solution. In some embodiments, the concentration of naloxone hydrochloride or hydrate thereof is greater than 12 mg per 100 μL of the aqueous solution. In some embodiments, the concentration of naloxone hydrochloride or hydrate thereof is about 8 mg per 100 μL of the aqueous solution. In some embodiments, the concentration of naloxone hydrochloride or hydrate thereof is about 10 mg per 100 μL to about 16 mg per 100 μL of the aqueous solution. In some embodiments, the concentration of naloxone hydrochloride or hydrate thereof is about 10 mg per 100 μL of the aqueous solution. In some embodiments, the concentration of naloxone hydrochloride or hydrate thereof is about 12 mg per 100 μL of the aqueous solution. In some embodiments, the concentration of naloxone hydrochloride or hydrate thereof is from about 2 mg per 100 μL to about 16 mg per 100 μL of the aqueous solution. In some embodiments, the concentration of naloxone hydrochloride or hydrate thereof is from about 2 mg per 100 μL to about 12 mg per 100 μL of the aqueous solution. In some embodiments, the concentration of naloxone hydrochloride or hydrate thereof is from about 2 mg per 100 μL to about 10 mg per 100 μL of the aqueous solution. In some embodiments, the concentration of naloxone hydrochloride or hydrate thereof is from about 4 mg per 100 μL to about 8 mg per 100 μL of the aqueous solution. In some embodiments, the concentration of naloxone hydrochloride or hydrate thereof is selected from the group consisting of about 4 mg per 100 μL, about 6 mg per 100 μL, and about 8 mg per 133 μL of the aqueous solution of the aqueous solution. In some embodiments, the concentration of naloxone hydrochloride or hydrate thereof is about 4 mg per 100 μL of the aqueous solution. In some embodiments, the concentration of naloxone hydrochloride or hydrate thereof is about 4.4 mg per 100 μL of the aqueous solution. In some embodiments, the concentration of naloxone hydrochloride or hydrate thereof is about 6 mg per 100 μL of the aqueous solution. In some embodiments, the concentration of naloxone hydrochloride or hydrate thereof is about 8 mg per 133 μL of the aqueous solution.

In some embodiments, the hydrate of naloxone hydrochloride is naloxone hydrochloride dihydrate.

In some embodiments, the concentration of chlorobutanol is from about 0.1 mg per 100 μL to about 0.8 mg per 100 μL of the aqueous solution. In some embodiments, the concentration of chlorobutanol is from about 0.2 mg per 100 μL to about 0.6 mg per 100 μL of the aqueous solution. In some embodiments, the concentration of chlorobutanol is from about 0.3 mg per 100 μL to about 0.5 mg per 100 μL of the aqueous solution. In some embodiments, the concentration of chlorobutanol is from about 0.4 mg per 100 μL to about 0.5 mg per 100 μL of the aqueous solution. In some embodiments, the concentration of chlorobutanol is about 0.4 mg per 100 μL of the aqueous solution. In some embodiments, the concentration of chlorobutanol is about 0.45 mg per 100 μL of the aqueous solution. In some embodiments, the concentration of chlorobutanol is about 0.53 mg per 100 μL of the aqueous solution. In some embodiments, the concentration of the acid is from about 0.001 mg per 100 μL to about 0.15 mg per 100 μL of the aqueous solution. In some embodiments, the concentration of the acid is from about 0.001 mg per 100 μL to about 0.05 mg per 100 μL of the aqueous solution. In some embodiments, the concentration of the acid is from about 0.001 mg per 100 μL to about 0.03 mg per 100 μL of the aqueous solution. In some embodiments, the concentration of the acid is from about 0.03 mg per 100 μL of the aqueous solution. In some embodiments, the concentration of the acid is from about 0.05 mg per 100 μL of the aqueous solution. In some embodiments, the concentration of the acid is no more than about 0.15 mg per 100 μL of the aqueous solution. In some embodiments, the concentration of the acid is from about 0.001 mg per 100 μL to about 0.15 mg per 100 μL of the aqueous solution. In some embodiments, the concentration of the acid is from about 0.002 mg per 100 μL to about 0.009 mg per 100 μL of the aqueous solution. In some embodiments, the concentration of the acid is from about 0.004 mg per 100 μL to about 0.009 mg per 100 μL of the aqueous solution. In some embodiments, the concentration of the acid is from about 0.005 mg per 100 μL to about 0.009 mg per 100 μL of the aqueous solution. In some embodiments, the concentration of the acid is from about 0.006 mg per 100 μL to about 0.009 mg per 100 μL of the aqueous solution. In some embodiments, the concentration of the acid is from about 0.006 mg per 100 μL to about 0.008 mg per 100 μL of the aqueous solution. In some embodiments, the concentration of the acid is about 0.009 mg per 100 μL of the aqueous solution.

In some embodiments, the concentration of the buffer is no more than about 0.15 mg per 100 μL of the aqueous solution. In some embodiments, the concentration of the buffer is from about 0.001 mg per 100 μL to about 0.15 mg per 100 μL of the aqueous solution. In some embodiments, the concentration of the buffer is from about 0.002 mg per 100 μL to about 0.03 mg per 100 μL of the aqueous solution. In some embodiments, the concentration of the buffer is from about 0.002 mg per 100 μL to about 0.02 mg per 100 μL of the aqueous solution. In some embodiments, the concentration of the buffer is from about 0.004 mg per 100 μL to about 0.015 mg per 100 μL of the aqueous solution. In some embodiments, the concentration of the buffer is from about 0.008 mg per 100 μL to about 0.012 mg per 100 μL of the aqueous solution. In some embodiments, the concentration of the buffer is from about 0.009 mg per 100 μL to about 0.011 mg per 100 μL of the aqueous solution. In some embodiments, the concentration of the buffer is about 0.01 mg per 100 μL of the aqueous solution. In some embodiments, the concentration of the buffer is about 0.019 mg per 100 μL of the aqueous solution. In some embodiments, the concentration of the buffer is about 0.02 mg per 100 μL of the aqueous solution. In some embodiments, the concentration of the isotonicity agent is no more than about 2 mg per 100 μL of the aqueous solution. In some embodiments, the concentration of the isotonicity agent is from about 0.1 mg per 100 μL to about 2 mg per 100 μL of the aqueous solution. In some embodiments, the concentration of the isotonicity agent is from about 0.4 mg per 100 μL to about 1.5 mg per 100 μL of the aqueous solution. In some embodiments, the concentration of the isotonicity agent is from about 0.5 mg per 100 μL to about 1 mg per 100 μL of the aqueous solution. In some embodiments, the concentration of the isotonicity agent is from about 0.7 mg per 100 μL to about 0.9 mg per 100 μL of the aqueous solution. In some embodiments, the concentration of the isotonicity agent is from about 0.8 mg per 100 μL to about 0.9 mg per 100 μL of the aqueous solution. In some embodiments, the concentration of the isotonicity agent is about 0.85 mg per 100 μL of the aqueous solution. In some embodiments, the concentration of the isotonicity agent is about 0.625 mg per 100 μL of the aqueous solution. In some embodiments, the concentration of the glycerin is from about 0.1 mg per 100 μL to about 2 mg per 100 μL of the aqueous solution. In some embodiments, the concentration of the glycerin is from about 0.4 mg per 100 μL to about 1.8 mg per 100 μL of the aqueous solution. In some embodiments, the concentration of the glycerin is from about 0.8 mg per 100 μL to about 1.6 mg per 100 μL of the aqueous solution. In some embodiments, the concentration of the glycerin is from about 1 mg per 100 μL to about 1.6 mg per 100 μL of the aqueous solution. In some embodiments, the concentration of the glycerin is from about 1.3 mg per 100 μL to about 1.5 mg per 100 μL of the aqueous solution. In some embodiments, the concentration of the glycerin is about 1.4 mg per 100 μL of the aqueous solution. In some embodiments, the osmolality of the composition is about 850 mOsm. In some embodiments, the osmolality of the composition is about 1000 mOsm. In some embodiments, the osmolality of the composition is from about 300 mOsm to about 700 mOsm. In some embodiments, the osmolality of the composition is from about 400 mOsm to about 700 mOsm. In some embodiments, the osmolality of the composition is from about 400 mOsm to about 650 mOsm. In some embodiments, the osmolality of the composition is about 446 mOsm. In some embodiments, the osmolality of the composition is about 614 mOsm. In some embodiments, the osmolality of the composition is about 613 mOsm. In some embodiments, the osmolality of the composition is about 607 mOsm. In some embodiments, the osmolality of the composition is about 446 mOsm and the composition does not comprise glycerin. In some embodiments, the osmolality of the composition is about 614 mOsm and the composition comprises glycerin. In some embodiments, the osmolality of the composition is about 607 mOsm and the composition comprises glycerin. In some embodiments, the osmolality of the composition is about 614 mOsm, the concentration of naloxone hydrochloride dihydrate is about 4 mg per 100 μL of aqueous solution, and the composition comprises glycerin. In some embodiments, the osmolality of the composition is about 613 mOsm, the concentration of naloxone hydrochloride dihydrate is about 4 mg per 100 μL of aqueous solution, and the composition comprises glycerin. In some embodiments, the osmolality of the composition is about 607 mOsm, the concentration of naloxone hydrochloride dihydrate is about 6 mg per 100 μL of aqueous solution, and the composition comprises glycerin. In some embodiments, the osmolality of the composition is about 607 mOsm, the concentration of naloxone hydrochloride dihydrate is about 8 mg per 133 μL of aqueous solution, and the composition comprises glycerin. In some embodiments, the pH of the composition is from about 3 to about 6. In some embodiments, the pH of the composition is from about 4 to about 5. In some embodiments, the pH of the composition is about 4.1. In some embodiments, the pH of the composition is about 4.0. In some embodiments, the composition is formulated for intranasal administration. In some embodiments, when intranasally administered to a subject, the pH of the composition is the pH of the nasal mucosa of the subject. In some embodiments, when intranasally administered to a subject, the pharmaceutical composition yields a naloxone Tₘₐₓ of less than 30 minutes. In some embodiments, when intranasally administered to a subject, the pharmaceutical composition yields a naloxone Tₘₐₓ of less than 25 minutes. In some embodiments, when intranasally administered to a subject, the pharmaceutical composition yields a naloxone Tₘₐₓ of less than 20 minutes. In some embodiments, when intranasally administered to a subject, the pharmaceutical composition yields a mean naloxone plasma concentration of ≥0.2 ng/mL within 2.5 minutes in said subject. In some embodiments, when intranasally administered to a subject, the pharmaceutical composition yields a mean naloxone plasma concentration of ≥1 ng/mL within 5 minutes in said subject. In some embodiments, when intranasally administered to a subject, the pharmaceutical composition yields a mean naloxone plasma concentration of ≥3 ng/mL within 10 minutes in said subject.

In one aspect, described herein is a pharmaceutical composition comprising naloxone hydrochloride or a hydrate thereof, chlorobutanol, citric acid, trisodium citrate dihydrate, sodium chloride, and glycerin.

In some embodiments, the hydrate of naloxone hydrochloride is naloxone hydrochloride dihydrate. In some embodiments, the concentration of naloxone hydrochloride dihydrate is selected from the group consisting of about 4 mg per 100 μL of composition, about 6 mg per 100 μL of composition, and about 8 mg per 133 μL of composition the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is selected from the group consisting of about 0.85 mg per 100 μL of composition and about 0.625 mg per 100 μL of composition, and the concentration of glycerin is about 1.4 mg per 100 μL of composition. In some embodiments, the concentration of naloxone hydrochloride dihydrate is about 4 mg per 100 μL of composition, the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is about 0.85 mg per 100 μL of composition, and the concentration of glycerin is about 1.4 mg per 100 μL of composition. In some embodiments, the concentration of naloxone hydrochloride dihydrate is selected from the group consisting of about 6 mg per 100 μL of composition, the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is selected from the group consisting of about 0.625 mg per 100 μL of composition, and the concentration of glycerin is about 1.4 mg per 100 μL of composition. In some embodiments, the concentration of naloxone hydrochloride dihydrate is selected from the group consisting of about 8 mg per 133 μL of composition the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is selected from the group consisting of about 0.625 mg per 100 μL of composition, and the concentration of glycerin is about 1.4 mg per 100 μL of composition.

In some embodiments, the pharmaceutical composition is an aqueous solution. In some embodiments, the volume of the aqueous solution is from about 50 μL to about 200 μL. In some embodiments, the volume of the aqueous solution is from about 80 μL to about 150 μL. In some embodiments, the volume of the aqueous solution is from about 90 μL to about 120 μL. In some embodiments, the volume of the aqueous solution is about 100 μL of the aqueous solution. In some embodiments, the volume of the aqueous solution is about 133 μL of the aqueous solution. In one aspect, provided herein is a pharmaceutical composition comprising naloxone hydrochloride or a hydrate thereof, morphine, hydrocodone, hydromorphone, oxycodone, oxymorphone, buprenorphine, levonaloxone, pseudomorphine, nalbutene, codeine, dextromethorophan, fentanyl, methadone, tramadol and other opioid antagonists; cyprodime, nalmefene, nalodeine, naloxol, naltrexone, or nalbuphene, and glycerin.

In some embodiments, the pharmaceutical composition comprises naloxone hydrochloride or a hydrate thereof and glycerin. In some embodiments, the pharmaceutical composition is an aqueous solution. In some embodiments, the concentration of glycerin is from about 0.5 mg per 100 μL of aqueous solution to about 2.5 mg per 100 μL of aqueous solution. In some embodiments, the concentration of the glycerin is from about 0.1 mg per 100 μL to about 2 mg per 100 μL of the aqueous solution. In some embodiments, the concentration of the glycerin is from about 0.4 mg per 100 μL to about 1.8 mg per 100 μL of the aqueous solution. In some embodiments, the concentration of the glycerin is from about 0.8 mg per 100 μL to about 1.6 mg per 100 μL of the aqueous solution. In some embodiments, the concentration of the glycerin is from about 1 mg per 100 μL to about 1.6 mg per 100 μL of the aqueous solution. In some embodiments, the concentration of the glycerin is from about 1.3 mg per 100 μL to about 1.5 mg per 100 μL of the aqueous solution. In some embodiments, the concentration of glycerin is about 2.5 mg per 100 μL of aqueous solution. In some embodiments, the concentration of glycerin is from about 2.5 mg per 100 μL of aqueous solution to about 5 mg per 100 μL of the aqueous solution. In some embodiments, the concentration of glycerin is about 1.4 mg per 100 μL of aqueous solution.

In some embodiments, the volume of the aqueous solution is from about 50 μL to about 200 μL. In some embodiments, the volume of the aqueous solution is from about 80 μL to about 150 μL. In some embodiments, the volume of the aqueous solution is from about 90 μL to about 120 μL. In some embodiments, the volume of the aqueous solution is about 100 μL of the aqueous solution. In some embodiments, the volume of the aqueous solution is 133 μL of the aqueous solution.

In some embodiments, the concentration of naloxone hydrochloride or a hydrate thereof is greater than 16 mg per 100 μL of aqueous solution. In some embodiments, the concentration of naloxone hydrochloride or a hydrate thereof is greater than 12 mg per 100 μL of aqueous solution. In some embodiments, the concentration of naloxone hydrochloride or a hydrate thereof is about 12 mg per 100 μL of aqueous solution. In some embodiments, the concentration of naloxone hydrochloride or a hydrate thereof is from about 1 mg per 100 μL of aqueous solution to about 12 mg per 100 μL of aqueous solution. In some embodiments, the concentration of naloxone hydrochloride or a hydrate thereof is from about 2 mg per 100 μL of aqueous solution to about 16 mg per 100 μL of aqueous solution. In some embodiments, the concentration of naloxone hydrochloride or hydrate thereof is from about 2 mg per 100 μL to about 10 mg per 100 μL of the aqueous solution. In some embodiments, the concentration of naloxone hydrochloride or hydrate thereof is from about 4 mg per 100 μL to about 8 mg per 100 μL of the aqueous solution. In some embodiments, the concentration of naloxone hydrochloride or hydrate thereof is selected from the group consisting of about 4 mg per 100 μL, about 6 mg per 100 μL, and about 8 mg per 133 μL of the aqueous solution of the aqueous solution. In some embodiments, the concentration of naloxone hydrochloride or hydrate thereof is about 4 mg per 100 μL of the aqueous solution. In some embodiments, the concentration of naloxone hydrochloride or hydrate thereof is about 4.4 mg per 100 μL of the aqueous solution. In some embodiments, the concentration of naloxone hydrochloride or hydrate thereof is about 6 mg per 100 μL of the aqueous solution. In some embodiments, the concentration of naloxone hydrochloride or hydrate thereof is about 8 mg per 133 μL of the aqueous solution.

In some embodiments, the hydrate of naloxone hydrochloride is naloxone hydrochloride dihydrate.

In some embodiments, the concentration of naloxone hydrochloride or hydrate thereof is from about 10 mg per 100 μL to about 16 mg per 100 μL of aqueous solution and the concentration of glycerin is about 1.4 mg 100 μL of aqueous solution. In some embodiments, the concentration of naloxone hydrochloride or hydrate thereof is from about 10 mg per 100 μL to about 16 mg per 100 μL of aqueous solution and the concentration of glycerin is about 2.5 mg 100 μL of aqueous solution.

In some embodiments, the concentration of naloxone hydrochloride or hydrate thereof is about 12 mg per 100 μL of aqueous solution and the concentration of glycerin is about 1.4 mg 100 μL of aqueous solution. In some embodiments, the concentration of naloxone hydrochloride or hydrate thereof is about 12 mg per 100 μL of aqueous solution and the concentration of glycerin is about 2.5 mg 100 μL of aqueous solution.

In some embodiments, the pharmaceutical composition comprises chlorobutanol. In some embodiments, the concentration of chlorobutanol is from about 0.1 mg per 100 μL to about 0.8 mg per 100 μL of the aqueous solution. In some embodiments, the concentration of chlorobutanol is from about 0.2 mg per 100 μL to about 0.6 mg per 100 μL of the aqueous solution. In some embodiments, the concentration of chlorobutanol is from about 0.3 mg per 100 μL to about 0.5 mg per 100 μL of the aqueous solution. In some embodiments, the concentration of chlorobutanol is from about 0.4 mg per 100 μL to about 0.5 mg per 100 μL of the aqueous solution. In some embodiments, the concentration of chlorobutanol is about 0.4 mg per 100 μL of the aqueous solution. In some embodiments, the concentration of chlorobutanol is about 0.45 mg per 100 μL of the aqueous solution. In some embodiments, the concentration of chlorobutanol is about 0.53 mg per 100 μL of the aqueous solution. In some embodiments, the concentration of the acid is from about 0.001 mg per 100 μL to about 0.15 mg per 100 μL of the aqueous solution. In some embodiments, the concentration of the acid is from about 0.001 mg per 100 μL to about 0.05 mg per 100 μL of the aqueous solution. In some embodiments, the concentration of the acid is from about 0.001 mg per 100 μL to about 0.03 mg per 100 μL of the aqueous solution. In some embodiments, the concentration of the acid is from about 0.03 mg per 100 μL of the aqueous solution. In some embodiments, the concentration of the acid is from about 0.05 mg per 100 μL of the aqueous solution. In some embodiments, the concentration of the acid is no more than about 0.15 mg per 100 μL of the aqueous solution. In some embodiments, the concentration of the acid is from about 0.001 mg per 100 μL to about 0.15 mg per 100 μL of the aqueous solution. In some embodiments, the concentration of the acid is from about 0.002 mg per 100 μL to about 0.009 mg per 100 μL of the aqueous solution. In some embodiments, the concentration of the acid is from about 0.004 mg per 100 μL to about 0.009 mg per 100 μL of the aqueous solution. In some embodiments, the concentration of the acid is from about 0.005 mg per 100 μL to about 0.009 mg per 100 μL of the aqueous solution. In some embodiments, the concentration of the acid is from about 0.006 mg per 100 μL to about 0.009 mg per 100 μL of the aqueous solution. In some embodiments, the concentration of the acid is from about 0.006 mg per 100 μL to about 0.008 mg per 100 μL of the aqueous solution. In some embodiments, the concentration of the acid is about 0.009 mg per 100 μL of the aqueous solution.

In some embodiments, the pharmaceutical composition comprises a buffer. In some embodiments, the buffer comprises, e.g., consists essentially of or consists of, citric acid and trisodium citrate. In some embodiments, the buffer comprises, e.g., consists essentially of or consists of, an acetate buffer. In some embodiments, the concentration of the buffer is no more than about 0.15 mg per 100 μL of the aqueous solution. In some embodiments, the concentration of the buffer is from about 0.001 mg per 100 μL to about 0.15 mg per 100 μL of the aqueous solution. In some embodiments, the concentration of the buffer is from about 0.002 mg per 100 μL to about 0.03 mg per 100 μL of the aqueous solution. In some embodiments, the concentration of the buffer is from about 0.002 mg per 100 μL to about 0.02 mg per 100 μL of the aqueous solution. In some embodiments, the concentration of the buffer is from about 0.004 mg per 100 μL to about 0.015 mg per 100 μL of the aqueous solution. In some embodiments, the concentration of the buffer is from about 0.008 mg per 100 μL to about 0.012 mg per 100 μL of the aqueous solution. In some embodiments, the concentration of the buffer is from about 0.009 mg per 100 μL to about 0.011 mg per 100 μL of the aqueous solution. In some embodiments, the concentration of the buffer is about 0.01 mg per 100 μL of the aqueous solution. In some embodiments, the concentration of the buffer is about 0.019 mg per 100 μL of the aqueous solution. In some embodiments, the concentration of the buffer is about 0.02 mg per 100 μL of the aqueous solution.

In some embodiments, the pharmaceutical composition comprises an isotonicity agent. In some embodiments, the isotonicity agent is sodium chloride. In some embodiments, the concentration of the isotonicity agent is no more than about 2 mg per 100 μL of the aqueous solution. In some embodiments, the concentration of the isotonicity agent is from about 0.1 mg per 100 μL to about 2 mg per 100 μL of the aqueous solution. In some embodiments, the concentration of the isotonicity agent is from about 0.4 mg per 100 μL to about 1.5 mg per 100 μL of the aqueous solution. In some embodiments, the concentration of the isotonicity agent is from about 0.5 mg per 100 μL to about 1 mg per 100 μL of the aqueous solution. In some embodiments, the concentration of the isotonicity agent is from about 0.7 mg per 100 μL to about 0.9 mg per 100 μL of the aqueous solution. In some embodiments, the concentration of the isotonicity agent is from about 0.8 mg per 100 μL to about 0.9 mg per 100 μL of the aqueous solution. In some embodiments, the concentration of the isotonicity agent is about 0.85 mg per 100 μL of the aqueous solution. In some embodiments, the concentration of the isotonicity agent is about 0.625 mg per 100 μL of the aqueous solution.

In some embodiments, the osmolality of the composition is about 850 mOsm. In some embodiments, the osmolality of the composition is about 1000 mOsm. In some embodiments, the osmolality of the composition is from about 300 mOsm to about 700 mOsm. In some embodiments, the osmolality of the composition is from about 400 mOsm to about 700 mOsm. In some embodiments, the osmolality of the composition is from about 400 mOsm to about 650 mOsm. In some embodiments, the osmolality of the composition is about 446 mOsm. In some embodiments, the osmolality of the composition is about 614 mOsm. In some embodiments, the osmolality of the composition is about 613 mOsm. In some embodiments, the osmolality of the composition is about 607 mOsm. In some embodiments, the osmolality of the composition is about 446 mOsm and the composition does not comprise glycerin. In some embodiments, the osmolality of the composition is about 614 mOsm and the composition comprises glycerin. In some embodiments, the osmolality of the composition is about 607 mOsm and the composition comprises glycerin. In some embodiments, the osmolality of the composition is about 614 mOsm, the concentration of naloxone hydrochloride dihydrate is about 4 mg per 100 μL of aqueous solution, and the composition comprises glycerin. In some embodiments, the osmolality of the composition is about 613 mOsm, the concentration of naloxone hydrochloride dihydrate is about 4 mg per 100 μL of aqueous solution, and the composition comprises glycerin. In some embodiments, the osmolality of the composition is about 607 mOsm, the concentration of naloxone hydrochloride dihydrate is about 6 mg per 100 μL of aqueous solution, and the composition comprises glycerin. In some embodiments, the osmolality of the composition is about 607 mOsm, the concentration of naloxone hydrochloride dihydrate is about 8 mg per 133 μL of aqueous solution, and the composition comprises glycerin.

In some embodiments, the pH of the composition is from about 3 to about 6. In some embodiments, the pH of the composition is from about 4 to about 5. In some embodiments, the pH of the composition is about 4.1. In some embodiments, the pH of the composition is about 4.0. In some embodiments, the composition is formulated for intranasal administration. In some embodiments, when intranasally administered to a subject, the pH of the composition is the pH of the nasal mucosa of the subject. In some embodiments, when intranasally administered to a subject, the pharmaceutical composition yields a naloxone Tₘₐₓ of less than 30 minutes. In some embodiments, when intranasally administered to a subject, the pharmaceutical composition yields a naloxone Tₘₐₓ of less than 25 minutes. In some embodiments, when intranasally administered to a subject, the pharmaceutical composition yields a naloxone Tₘₐₓ of less than 20 minutes. In some embodiments, when intranasally administered to a subject, the pharmaceutical composition yields a mean naloxone plasma concentration of ≥0.2 ng/mL within 2.5 minutes in said subject. In some embodiments, when intranasally administered to a subject, the pharmaceutical composition yields a mean naloxone plasma concentration of ≥1 ng/mL within 5 minutes in said subject. In some embodiments, when intranasally administered to a subject, the pharmaceutical composition yields a mean naloxone plasma concentration of ≥3 ng/mL within 10 minutes in said subject.

In one aspect, provided herein is a pharmaceutical composition comprising naloxone hydrochloride or a hydrate thereof, e.g., naloxone hydrochloride dihydrate, chlorobutanol, trisodium citrate dihydrate, and glycerin. In some embodiments the pharmaceutical composition is an aqueous solution. In some embodiments, the volume of the aqueous solution is about 100 μL. In some embodiments, the pharmaceutical composition comprises about 13.2 mg of naloxone hydrochloride dihydrate per 100 μL of aqueous solution, about 0.53 mg of chlorobutanol hemihydrate per 100 μL of aqueous solution, about 0.009 mg of anhydrous citric acid per 100 μL of aqueous solution about 0.015 mg of trisodium citrate dihydrate per 100 μL of aqueous solution, and about 1.4 mg of glycerin per 100 μL of aqueous solution. In some embodiments, the pH of the aqueous solution is from about 3.5 to about 4.7. In some embodiments, the pH of the aqueous solution is about 4.1. In some embodiments, the osmolality of the pharmaceutical composition is from about 400 mOsm to about 850 mOsm. In some embodiments, the osmolality of the pharmaceutical composition is about 615 mOsm. In some embodiments the density of the aqueous solution is about 1.04 mg/mL.

In some embodiments, the composition is formulated for intranasal administration. In some embodiments, when intranasally administered to a subject, the pH of the composition is the pH of the nasal mucosa of the subject. In some embodiments, when intranasally administered to a subject, the pharmaceutical composition yields a naloxone Tₘₐₓ of less than 30 minutes. In some embodiments, when intranasally administered to a subject, the pharmaceutical composition yields a naloxone Tₘₐₓ of less than 25 minutes. In some embodiments, when intranasally administered to a subject, the pharmaceutical composition yields a naloxone Tₘₐₓ of less than 20 minutes. In some embodiments, when intranasally administered to a subject, the pharmaceutical composition yields a mean naloxone plasma concentration of ≥0.2 ng/mL within 2.5 minutes in said subject. In some embodiments, when intranasally administered to a subject, the pharmaceutical composition yields a mean naloxone plasma concentration of ≥1 ng/mL within 5 minutes in said subject. In some embodiments, when intranasally administered to a subject, the pharmaceutical composition yields a mean naloxone plasma concentration of ≥3 ng/mL within 10 minutes in said subject.

In one aspect, described herein is a method of treating an opioid overdose or symptom thereof in a subject in need thereof, comprising administering to the subject a pharmaceutical composition described herein.

In some embodiments, the pharmaceutical composition comprises a therapeutically effective amount of naloxone hydrochloride or a hydrate thereof. In some embodiments, the hydrate of naloxone hydrochloride is naloxone hydrochloride dihydrate. In some embodiments, the therapeutically effective amount is from about 2 mg to about 16 mg. In some embodiments, the therapeutically effective amount is from about 2 mg to about 12 mg. In some embodiments, the therapeutically effective amount is from about 2 mg to about 10 mg. In some embodiments, the therapeutically effective amount is from about 4 mg to about 8 mg. In some embodiments, the therapeutically effective amount is selected from the group consisting of about 4 mg, about 6 mg, and about 8 mg. In some embodiments, the therapeutically effective amount is about 4 mg. In some embodiments, the therapeutically effective amount is about 6 mg. In some embodiments, the therapeutically effective amount is about 8 mg.

In some embodiments, upon nasal delivery of said pharmaceutical composition to said subject, less than about 20% of said pharmaceutical composition leaves the nasal cavity via drainage into the nasopharynx or externally. In some embodiments, the upon nasal delivery of said pharmaceutical composition to said subject, less than about 10% of said pharmaceutical composition leaves the nasal cavity via drainage into the nasopharynx or externally. In some embodiments, the upon nasal delivery of said pharmaceutical composition to said subject, less than about 5% of said pharmaceutical composition leaves the nasal cavity via drainage into the nasopharynx or externally.

In some embodiments, the plasma concentration versus time curve of said naloxone hydrochloride or hydrate thereof in said subject has a Tₘₐₓ of between about 20 and about 30 minutes. In some embodiments, the administration yields a mean naloxone plasma concentration of ≥0.2 ng/mL within 2.5 minutes in said subject. In some embodiments, the administration yields a mean naloxone plasma concentration of ≥1 ng/mL within 5 minutes in said subject. In some embodiments, the administration yields a mean naloxone plasma concentration of ≥3 ng/mL within 10 minutes in said subject.

In some embodiments, the subject exhibits one or more symptoms selected from the group consisting of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting. In some embodiments, the subject exhibits respiratory depression. In some embodiments, the opioid overdose or symptom thereof is caused by an opioid selected from the group consisting of codeine, morphine, methadone, fentanyl, carfentanyl, acetyl fentanyl, oxycodone hydrochloride, hydrocodone bitartrate, hydromorphone, oxymorphone, meperidine, propoxyphene, opium, heroin, tramadol, tapentadol, and narcotic-antagonist analgesics. In some embodiments, the narcotic-antagonist analgesics is selected from the group consisting of nalbuphine, pentazocine, and butorphanol. In some embodiments, the subject is a mammal. In some embodiments, the subject is a human. In some embodiments, the subject is an opioid overdose subject or a suspected opioid overdose subject.

In one aspect, described herein is a device configured for intranasally administering of a pharmaceutical composition described herein, wherein the device is configured for delivery of one dose of the pharmaceutical composition to the subject.

In some embodiments, the dose is contained in a single reservoir. In some embodiments, the device is adapted for single use. In some embodiments, the device is configured for delivery of the dose to the subject by a single actuation. In some embodiments, the device is not primed prior administering the dose to the subject.

In some embodiments, the volume of said reservoir is not more than about 200 μL. In some embodiments, the volume of said reservoir is not more than about 140 μL. In some embodiments, the volume of the single dose in the reservoir is from about 90 μL to about 140 μL. In some embodiments, the volume of the single dose in the reservoir is from about 95 μL to about 135 μL. In some embodiments, the volume of the single dose in the reservoir is about 100 μL. In some embodiments, the volume of the single dose in the reservoir is about 133 μL. In some embodiments, the device is configured to deliver the single dose at a volume from about 90 μL to about 140 μL. In some embodiments, the device is configured to deliver the single dose at a volume from about 95 μL to about 135 μL. In some embodiments, the device is configured to deliver the single dose at a volume of about 100 μL. In some embodiments, the device is configured to deliver the single dose at a volume of about 133 μL.

In some embodiments, the device is actuatable with one hand. In some embodiments, the device is configured such that the 90% confidence interval for dose delivered per actuation is ±about 2%. In some embodiments, the device is configured such that the 95% confidence interval for dose delivered per actuation is ±about 2.5%. In some embodiments, the device is configured such that the delivery time is less than about 25 seconds. In some embodiments, the device is configured such that the delivery time is less than about 20 seconds.

In some embodiments, upon nasal delivery of said pharmaceutical composition to said subject, less than about 20% of said pharmaceutical composition leaves the nasal cavity via drainage into the nasopharynx or externally. In some embodiments, upon nasal delivery of said pharmaceutical composition to said subject, less than about 10% of said pharmaceutical composition leaves the nasal cavity via drainage into the nasopharynx or externally. In some embodiments, upon nasal delivery of said pharmaceutical composition to said subject, less than about 5% of said pharmaceutical composition leaves the nasal cavity via drainage into the nasopharynx or externally. In some embodiments, the plasma concentration versus time curve of the naloxone hydrochloride or hydrate thereof in said subject has a Tₘₐₓ of between about 10 and about 30 minutes.

In some embodiments, the subject exhibits one or more symptoms selected from the group consisting of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting. In some embodiments, the subject exhibits respiratory depression. In some embodiments, said respiratory depression is caused by the illicit use of opioids, or by an accidental misuse of opioids during medical opioid therapy. In some embodiments, said subject is free from respiratory depression for at least about 1 hour following treatment comprising essentially of delivery of said therapeutically effective amount of said opioid antagonist. In some embodiments, said subject is free from respiratory depression for at least about 2 hours following treatment comprising essentially of delivery of said therapeutically effective amount of said opioid antagonist. In some embodiments, said subject is free from respiratory depression for at least about 4 hours following treatment comprising essentially of delivery of said therapeutically effective amount of said opioid antagonist. In some embodiments, said subject is free from respiratory depression for at least about 6 hours following treatment comprising essentially of delivery of said therapeutically effective amount of said opioid antagonist. In some embodiments, said subject is in a lying, supine, or recovery position.

In some embodiments, said single actuation yields a plasma concentration of ≥0.2 ng/mL within 2.5 minutes in said subject. In some embodiments, said single actuation yields a plasma concentration of ≥1 ng/mL within 5 minutes in said subject. In some embodiments, said single actuation yields a plasma concentration of ≥3 ng/mL within 10 minutes in said subject. In some embodiments, said single actuation yields a plasma concentration of ≥0.2 ng/mL within 2.5 minutes in said subject. In some embodiments, said single actuation yields a plasma concentration of ≥1 ng/mL within 5 minutes in said subject. In some embodiments, the subject is a mammal. In some embodiments, the subject is a human. In some embodiments, said subject is an opioid overdose subject or a suspected opioid overdose subject.

In some embodiments, the pharmaceutical composition is administered as a spray of droplets to the subject. In some embodiments, the Dv90 of the spray of droplets is from about 48 μm to about 80 μm. In some embodiments, the Dv90 of the spray of droplets is from about 60 μm to about 80 μm. In some embodiments, the Dv90 of the droplets is from about 56.77 μm. In some embodiments, the Dv90 of the droplets is from about 55.10 μm. In some embodiments, the Dv50 of the droplets is from about 20 μm to about 40 μm. In some embodiments, the Dv50 of the droplets is from about 25 μm to about 40 μm. In some embodiments, the Dv50 of the droplets is from about 22 μm to about 34 μm. In some embodiments, the Dv50 of the droplets is from about 24.72 μm. In some embodiments, the Dv50 of the droplets is from about 24.75 μm. In some embodiments, the Dv10 of the droplets is from about 10 μm to about 20 μm. In some embodiments, the Dv10 of the droplets is from about 12 μm to about 20 μm. In some embodiments, the Dv10 of the droplets is from about 10 μm to about 17 μm. In some embodiments, the Dv10 of the droplets is about 11.35 μm. In some embodiments, the Dv10 of the droplets is about 11.47 μm. In some embodiments, the percent volume of droplets less than 10 μm is less than about 12%. In some embodiments, the percent volume of droplets less than 10 μm is less than about 10%. In some embodiments, the percent volume of droplets less than 10 μm is about 6.7%. In some embodiments, the percent volume of droplets less than 10 μm is about 6.3%. In some embodiments, the device sprays a spray pattern with a Dmax of about 50 mm. In some embodiments, the device sprays a spray pattern with a Dmax of about 40.2 mm. In some embodiments, the device sprays a spray pattern with a Dmax of about 40.7 mm. In some embodiments, the device sprays a spray pattern with an area of about 750 mm² to about 1500 mm². In some embodiments, the device sprays a spray pattern with an area of about 1100 mm². In some embodiments, the device sprays a spray pattern with an area of about 1110 mm². In some embodiments, the device sprays a spray pattern with an ovality ratio of about 1.0 to about 2.5. In some embodiments, the device sprays a spray pattern with an ovality ratio of about 1.2.

In one aspect, described herein is a method of treating an opioid overdose or a symptom thereof, comprising intranasally administering to the subject a pharmaceutical composition comprising greater than 4 mg of naloxone hydrochloride or a hydrate thereof and chlorobutanol.

In one aspect, described herein is a method of treating an opioid overdose or a symptom thereof, comprising intranasally administering to the subject a pharmaceutical composition comprising greater than 4 mg of naloxone hydrochloride or a hydrate thereof and glycerin.

In some embodiments, the pharmaceutical composition is an aqueous solution. In some embodiments, the pharmaceutical composition is administered to the subject in a single dose. In some embodiments, the concentration of naloxone hydrochloride is about 6 mg per 100 μL of aqueous solution. In some embodiments, the total mass of naloxone hydrochloride administered is not more than about 20 mg. In some embodiments, the total mass of the hydrate of naloxone hydrochloride administered is not more than about 20 mg. In some embodiments, the total administered volume is from about 30 μL to about 200 μL. In some embodiments, the total administered volume is from about 50 μL to about 150 μL.

### BRIEF DESCRIPTION OF THE DRAWINGS

FIG. 1 depicts the compositions of exemplary pharmaceutical formulations comprising naloxone hydrochloride and placebo.

FIG. 2 depicts an exemplary plan of stability tests of the formulations described in FIG. 1.

FIG. 3 depicts exemplary analytical data from the initial point of the stability studies of the formulations described in FIG. 1.

### DETAILED DESCRIPTION

The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

The present subject matter will now be described more fully hereinafter with reference to the accompanying Figures and Examples, in which representative embodiments are shown. The present subject matter can, however, be embodied in different forms and should not be construed as limited to the embodiments set forth herein. Rather, these embodiments are provided to describe and enable one of skill in the art. Unless otherwise defined, all technical and scientific terms used herein have the same meaning as commonly understood by one of ordinary skill in the art to which the subject matter pertains. All publications, patent applications, patents, and other references mentioned herein are incorporated by reference in their entirety.

### Definitions

To facilitate the understanding of the present invention, a number of terms and phrases are defined below.

The terms “a” and “an” as used herein mean “one or more” and include the plural unless the context is inappropriate.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “active ingredient” or “pharmaceutically active compound” is defined in the context of a “pharmaceutical composition” and is intended to mean a component of a pharmaceutical composition that provides the primary pharmacological effect, as opposed to an “inactive ingredient” which would generally be recognized as providing no pharmaceutical benefit.

The term “actuation,” as used herein, refers to operation of the device such that the pharmaceutical composition is delivered therefrom.

The term “agonist,” as used herein, refers to as used herein refers to a moiety that interacts with and activates a receptor, and thereby initiates a physiological or pharmacological response characteristic of that receptor.

The term “antagonist,” as used herein, refers to a moiety that competitively binds to a receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist.

The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist.

The term “antimicrobial preservative,” as used herein, refers to a pharmaceutically acceptable excipient with antimicrobial properties which is added to a pharmaceutical composition to maintain microbiological stability.

The term “AUC,” as used herein, refers to the area under the drug plasma concentration-time curve.

The term “AUC₀₋ₜ,” as used herein, refers to the area under the drug plasma concentration-time curve from t=0 to the last measurable concentration.

The term “AUC_{0-∞},” as used herein, refers to the area under the drug plasma concentration-time curve extrapolated to ∞.

The term “AUC_{0-t/D},” as used herein, refers to the AUC₀₋ₜ normalized to 0.4 mg IM naloxone.

The term “AUC_{0-∞/D},” as used herein, refers to the AUC_{0-∞} normalized to 0.4 mg IM naloxone.

As used herein, the term “aqueous solution” refers to a composition comprising in whole, or in part, water.

The term “bioavailability (F),” as used herein, refers to the fraction of a dose of drug that is absorbed from its site of administration and reaches, in an unchanged form, the systemic circulation.

The term “absolute bioavailability” is used when the fraction of absorbed drug is related to its IV bioavailability. It may be calculated using the following formula:

$$F = \frac{{AUC}_{extravascular}}{{AUC}_{intravenous}} \times \frac{{Dose}_{intravenous}}{{Dose}_{extravascular}}$$

The term relative bioavailability (Fᵣₑₗ) is used to compare two different extravascular routes of drug administration and it may be calculated using the following formula:

$${F}_{rel} = \frac{{AUC}_{extravascular 1}}{{AUC}_{extravascular 2}} \times \frac{{Dose}_{extravascular 2}}{{Dose}_{extravascular 1}}$$

As used herein, the terms “buffer,” “buffer system,” or “buffering component” refers to a compound that, usually in combination with at least one other compound, provides a chemical system in solution that exhibits buffering capacity, that is, the capacity to neutralize, within limits, the pH lowering or raising effects of either strong acids or bases (alkali), respectively, with relatively little or no change in the original pH (e.g., the pH before being affected by, e.g., strong acid or base). For example, a buffer described herein maintains or controls the pH of a solution to a certain pH range. For example, “buffering capacity” can refer to the millimoles (mM) of strong acid or base (or respectively, hydrogen or hydroxide ions) required to change the pH by one unit when added to one liter (a standard unit) of the buffer solution. From this definition, it is apparent that the smaller the pH change in a solution caused by the addition of a specified quantity of acid or alkali, the greater the buffer capacity of the solution. See, for example, Remington: The Science and Practice of Pharmacy, Mack Publishing Co., Easton, Pa. (19^{th} Edition, 1995), Chapter 17, pages 225-227. The buffer capacity will depend on the kind and concentration of the buffer components.

The term “clearance (CL),” as used herein, refers to the rate at which a drug is eliminated divided by its plasma concentration, giving a volume of plasma from which drug is completely removed per unit of time. CL is equal to the elimination rate constant (λ) multiplied by the volume of distribution (V_{d}), wherein “V_{d}” is the fluid volume that would be required to contain the amount of drug present in the body at the same concentration as in the plasma.

The term “apparent clearance (CL/F),” as used herein, refers to clearance that does not take into account the bioavailability of the drug. It is the ratio of the dose over the AUC.

The term “Cₘₐₓ,” as used herein, refers to the maximum observed plasma concentration.

The term “C_{max/D},” as used herein, refers to Cₘₐₓ normalized to 0.4 mg IM naloxone.

The term “coefficient of variation (CV),” as used herein, refers to the ratio of the sample standard deviation to the sample mean. It is often expressed as a percentage.

The term “confidence interval,” as used herein, refers to a range of values which will include the true average value of a parameter a specified percentage of the time.

The term “device,” as used herein, refers to an apparatus capable of delivering a drug to patient in need thereof.

The term “delivery time,” as used herein, refers to the amount of time that elapses between a determination made by a healthcare professional, or an untrained individual that an individual is in need of nasal delivery of an opioid antagonist and completion of the delivery.

The term “elimination rate constant (λ),” as used herein, refers to the fractional rate of drug removal from the body. This rate is constant in first-order kinetics and is independent of drug concentration in the body. λ is the slope of the plasma concentration-time line (on a logarithmic y scale).

The term “λ_{z}” as used herein, refers to the terminal phase elimination rate constant, wherein the “terminal phase” of the drug plasma concentration-time curve is a straight line when plotted on a semilogarithmic graph. The terminal phase is often called the “elimination phase” because the primary mechanism for decreasing drug concentration during the terminal phase is drug elimination from the body. The distinguishing characteristic of the terminal elimination phase is that the relative proportion of drug in the plasma and peripheral volumes of distribution remains constant. During this “terminal phase” drug returns from the rapid and slow distribution volumes to the plasma, and is permanently removed from the plasma by metabolism or renal excretion.

The term “equivalent,” as used herein refers to a weight of an opioid antagonist selected from naloxone and pharmaceutically acceptable salts thereof that is equimolar to a specified weight of naloxone hydrochloride. For example, 8 mg of anhydrous naloxone hydrochloride (molecular weight, 363.84) is equivalent to about 7.2 mg of naloxone freebase (molecular weight, 327.37), and to about 8.8 mg of naloxone hydrochloride dihydrate (molecular weight 399.87).

The term “filled,” as used herein, refers to an association between a device and a pharmaceutical composition, for example, when a pharmaceutical composition described herein comprising a therapeutically effective amount of an opioid antagonist is present within a reservoir that forms a part of a device described herein.

The term “hydrate,” as used herein, refers to an opioid antagonist described herein or a salt thereof that further includes a stoichiometric or non-stoichiometric amount of water bound by non-covalent intermolecular forces.

The term “in need of treatment” and the term “in need thereof” when referring to treatment are used interchangeably and refer to a judgment made by a caregiver (e.g. physician, nurse, nurse practitioner, that a patient will benefit from treatment.

As used herein, two embodiments are “mutually exclusive” when one is defined to be something which is different than the other. For example, an embodiment wherein the amount of naloxone hydrochloride is specified to be 4 mg is mutually exclusive with an embodiment wherein the amount of naloxone hydrochloride is specified to be 2 mg. However, an embodiment wherein the amount of naloxone hydrochloride is specified to be 4 mg is not mutually exclusive with an embodiment in which less than about 10% of said pharmaceutical composition leaves the nasal cavity via drainage into the nasopharynx or externally.

As used herein, “spray pattern” is the shape of the plume when looking downward on the nasal spray unit as the product is emitted from the nasal spray unit.

As used herein, “ovality” or “ovality ratio” is the ratio of Dmax/Dmin, where Dmax and Dmin are the length of the longest and shortest line, respectively, that passes through the weighted center of mass drawn within the parameter of the spray pattern. In some embodiments, Dmax and Dmin are in units of mm.

The term “glycerin” refers to the compound propane-1,2,3-triol, which has the structure:

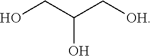

The term “naloxone,” as used herein, refers to a compound of the following structure:

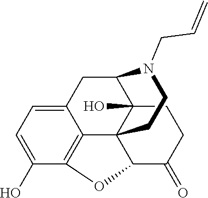

or a pharmaceutically acceptable salt, hydrate, or solvate thereof. The CAS registry number for naloxone is 465-65-6. Other names for naloxone include: 17-allyl-4,5a-epoxy-3,14-dihydroxymorphinan-6-one; (-)-17-allyl-4,5a-epoxy-3,14-dihydroxymorphinan-6-one; 4,5a-epoxy-3,14-dihydroxy-17-(2-propenyl)morphinan-6-one; and (-)-12-allyl-7,7a,8,9-tetrahydro-3,7a-dihydroxy-4aH-8,9c-iminoethanophena-nthro[4,5-bcd]furan-5(6H)-one. Naloxone hydrochloride may be anhydrous (CAS Reg. No. 357-08-4) and also forms a dihydrate (CAS No. 51481-60-8). It has been sold under various brand names including NARCAN™ NALONE™, NALOSSONE™, NALOXONA™, NALOXONUM™, NARCANTI™, and NARCON™.

The term “naltrexone,” as used herein, refers to a compound of the following structure:

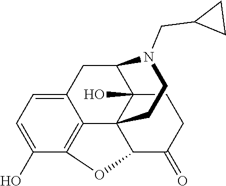

or a pharmaceutically acceptable salt, hydrate, or solvate thereof. The CAS registry number for naltrexone is 16590-41-3. Other names for naltrexone include: 17-(cyclopropylmethyl)-4,5a-epoxy-3,14-dihydroxymorphinan-6-one; (5a)-17-(cyclopropylmethyl)-3,14-dihydroxy-4,5-epoxymorphinan-6-one; and (1S,5R,13R,17S)-4-(cyclopropylmethyl)-10,17-dihydroxy-12-oxa-4-azapentacy-clo[9.6.1.01,13.05,17.07,18]octadeca-7(18),8,10-trien-14-one. Naltrexone hydrochloride (CAS Reg. No. 16676-29-2) has been marketed under the trade names ANTAXONE™, DEPADE™, NALOREX™, REVIA™, TREXANr™ VIVITREX™, and VIVITROL™.

The term “methylnaltrexone,” as used herein, refers to a pharmaceutically acceptable salt comprising the cation (5a)-17-(cyclopropylmethyl)-3,14-dihydroxy-17-methyl-4,5-epoxymorphinaniu-m-17-ium-6-one a compound of the following structure:

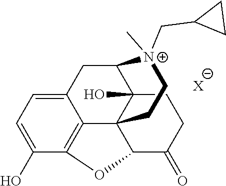

wherein X^{⊖} is a pharmaceutically acceptable anion. Methylnaltrexone bromide (CAS Reg. No. 75232-52-7) has been marketed under the trade name RELISTOR™.

The term “nalmefene,” as used herein, refers to 17-cyclopropylmethyl-4,5a-epoxy-6-methylenemorphinan-3,14-diol, a compound of the following structure:

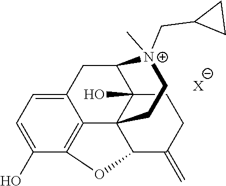

Nalmefene hydrochloride (CAS Reg. No. 58895-64-0) has been marketed under the trade names NALMETRENE™, CERVENE™, REVEX™, ARTHRENE™, and INCYSTENE™.

“Chlorobutanol” as used herein refers to the compound having the structure

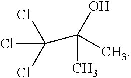

Chlorobutanol as defined herein (1,1,1-trichloro-2-methylpropan-2-ol; CAS Reg. No. 57-15-8) is a known preservative that can be used as an antibacterial and/or antifungal agent in pharmaceutical and cosmetic formulations. In an embodiment, the chlorobutanol is anhydrous. In another embodiment, the chlorobutanol is a solvate, e.g., a hydrate such as a hemihydrate.

The term “fentanyl derivative” as used herein refers to a molecule of Formula (I)

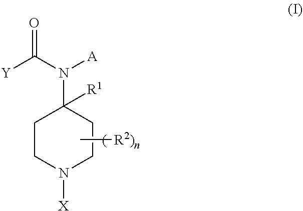

wherein A is aryl or heteroaryl optionally substituted with halo, C₁-C₃ alkyl, or C₁-C₃ alkoxy, X is C₁-C₃ alkyl or hydroxyethyl, optionally substituted with —COOCH₃, aryl, or heteroaryl optionally substituted with both C₁-C₃ alkyl and ═O, Y is C₁-C₄ alkyl, C₂-C₃ alkenyl, C₁-C₃ alkoxy, C₁-C₃ alkoxyalkyl, cycloalkyl, or heteroaryl, R¹ and R² are each independently selected from the group consisting of phenyl, C₁-C₃ alkyl, C₂-C₃ alkenyl, C₁-C₃ alkoxyalkyl, or C₁-C₃ alkoxy, and —COOCH₃, and n is 1, 2, or 3.

The term “nostril,” as used herein, is synonymous with “naris.”

The term “opioid antagonist” includes, in addition to naloxone and pharmaceutically acceptable salts thereof: naltrexone, methylnaltrexone, and nalmefene, and pharmaceutically acceptable salts thereof. In some embodiments, the opioid antagonist is naloxone hydrochloride. In some embodiments, the opioid antagonist is naloxone hydrochloride dihydrate. In some embodiments, the opioid antagonist is naltrexone hydrochloride. In some embodiments, the opioid antagonist is methylnaltrexone bromide. In some embodiments, the opioid antagonist is nalmefene hydrochloride. In some embodiments, the nasally administering is accomplished using a device described herein.

The term “opioid overdose,” as used herein, refers to an acute medical condition induced by excessive use of one or more opioids. Symptoms of opioid overdose include respiratory depression (including postoperative opioid respiratory depression, acute lung injury, and aspiration pneumonia), central nervous system depression (which may include sedation, altered level consciousness, miotic (constricted) pupils), and cardiovascular depression (which may include hypoxemia and hypotension). Visible signs of opioid overdose or suspected opioid overdose include: unresponsiveness and/or loss of consciousness (won't respond to stimuli such as shouting, shaking, or rubbing knuckles on sternum); slow, erratic, or stopped breathing; slow, erratic, or stopped pulse; deep snoring or choking/gurgling sounds; blue or purple fingernails or lips; pale and/or clammy face; slack or limp muscle tone; contracted pupils; and vomiting. Because opioid overdose may be difficult to diagnose and/or quantify, particularly by a lay person, as used herein, treatment of opioid overdose is meant to include treatment of suspected opioid overdose in opioid-intoxicated patients. Opioids that may induce overdose include, codeine, morphine, methadone, fentanyl, oxycodone HCl, hydrocodone bitartrate, hydromorphone, oxymorphone, meperidine, propoxyphene, opium, heroin, tramadol, tapentadol, and certain narcotic-antagonist analgesics, such as, nalbuphine, pentazocine and butorphanol. In some embodiments, the opioid agonist is in a tamper-proof formulation. In some embodiments, the opioid agonist is in a tamper-resistant formulation. In some embodiments, the opioid agonist is selected from ACUROX™, Oxycodone DETERx™, Egalet hydrocodone, Egalet morphine, Egalet oxycodone, EXALGO™, OPANA™, and REMOXY™.

The term “patient,” as used herein, refers to any subject (preferably human) afflicted with a condition likely to benefit from a treatment with a therapeutically effective amount of an opioid antagonist.

The term “pharmaceutical composition,” as used herein, refers to a composition comprising at least one active ingredient; including but not limited to, salts, solvates and hydrates of the opioid antagonists described herein, whereby the composition is amenable to use for a specified, efficacious outcome in a mammal (for example, without limitation, a human).

The term “prone,” as used herein, refers to a patient who is lying face down.

The term “receptor binding or occupancy” refers to a characterization of the kinetics between a radioactive drug and receptors or other binding sites throughout the body, and characterization of the radioactive drug binding affinity to these receptors.

The term “recovery position,” as used herein, means a position of the human body in which a patient lies on his/her side, with a leg or knee out in front (e.g., to prevent rolling onto his/her stomach) and at least one hand supporting the head (e.g., to elevate the face to facilitate breathing and prevent inhalation of vomit).

The term “solvate,” as used herein, refers to an opioid antagonist described herein or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “sterile filling,” as used herein, refers methods of manufacturing the devices and pharmaceutical compositions described herein, such that the use of preservatives is not required. Sterile drug products may be produced using aseptic processing or terminal sterilization. Terminal sterilization usually involves filling and sealing product containers under high-quality environmental conditions. In an aseptic process, the drug product, container, and closure are first subjected to sterilization methods separately, as appropriate, and then brought together.

The term “storage-stable,” as used herein, refers to a pharmaceutical composition in which at least about 95% to 99.5% of the active ingredient remains in an undegraded state after storage of the pharmaceutical composition at specified temperature and humidity for a specified time, for example, for 12 months at 25° C. and 60% relative humidity.

The term “supine,” as used herein, refers to a patient who is lying face up.

The term “t_{1/2}” or “half-life,” as used herein, refers to the amount of time required for half of a drug to be eliminated from the body or the time required for a drug concentration to decline by half.

The term “tonicity agent,” as used herein, refers to a compound which modifies the osmolality of a formulation, for example, to render it isotonic. Tonicity agents include, dextrose, lactose, sodium chloride, calcium chloride, magnesium chloride, sorbitol, sucrose, mannitol, trehalose, raffinose, polyethylene glycol, hydroxyethyl starch, glycine and the like.

The term “tomography,” as used herein, refers to a process of imaging by sections. The images may be looked at individually, as a series of two-dimensional slices or together, as a computer-generated three-dimensional representation.

The term “pharmaceutically acceptable,” as used herein, refers to a component of a pharmaceutical composition that it compatible with the other ingredients of the formulation and not overly deleterious to the recipient thereof.

The term “therapeutically effective amount,” as used herein, refers to the amount of active compound or pharmaceutical agent that elicits the biological or medicinal response in a tissue, system, or individual that is being sought by a researcher, healthcare provider or individual.

The term “Tₘₐₓ,” as used herein, refers to the time from administration of the pharmaceutical compositions described herein to maximum drug plasma concentration.

The term “untrained individual” refers to an individual administering to patient an opioid antagonist using a device described herein, wherein the individual is not a healthcare professional and has received no training in the use of the device, such as through an overdose education and nasal naloxone distribution (OEND) program.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 8 carbon atoms (“C₁₋₈ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

“Alkoxy” refers to the group —OR^{AB} where R^{AB} is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e., with between 1 and 6 carbon atoms. Further particular alkoxy groups have between 1 and 4 carbon atoms.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁4 aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Aryl groups include, but are not limited to, phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following formulae:

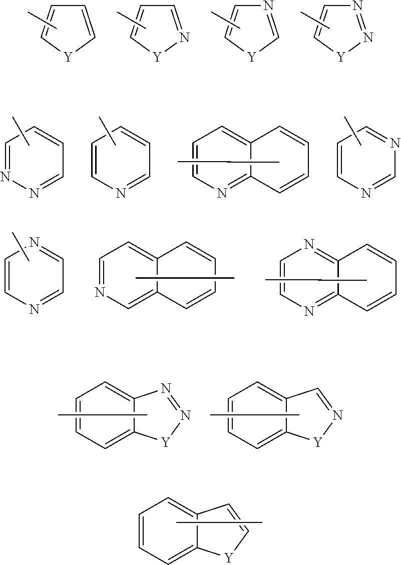

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Carbocyclyl” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₆ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “Cycloalkyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

Alkyl, alkoxyalkyl, carbocyclyl, cycloalkyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

### Opioid Antagonists

Provided herein are drug products adapted for nasal delivery of an opioid receptor antagonist. Opioid receptor antagonists are a well-recognized class of chemical agents. They have been described in detail in the scientific and patent literature. Pure opioid antagonists, such as naloxone, are agents which specifically reverse the effects of opioid agonists but have no opioid agonist activity.

Naloxone is commercially available as a hydrochloride salt. Naloxone hydrochloride (17-allyl-4,5a-epoxy-3,14-dihydroxymorphinan-6-one hydrochloride), a narcotic antagonist, is a synthetic congener of oxymorphone. In structure it differs from oxymorphone in that the methyl group on the nitrogen atom is replaced by an allyl group. Naloxone hydrochloride is an essentially pure narcotic antagonist, i.e., it does not possess the “agonistic” or morphine-like properties characteristic of other narcotic antagonists; naloxone does not produce respiratory depression, psychotomimetic effects or pupillary constriction. In the absence of narcotics or agonistic effects of other narcotic antagonists it exhibits essentially no pharmacologic activity. Naloxone has not been shown to produce tolerance or to cause physical or psychological dependence. In the presence of physical dependence on narcotics naloxone will produce withdrawal symptoms.

While the mechanism of action of naloxone is not fully understood, the preponderance of evidence suggests that naloxone antagonizes the opioid effects by competing for the same receptor sites. When naloxone hydrochloride is administered intravenously the onset of action is generally apparent within two minutes; the onset of action is only slightly less rapid when it is administered subcutaneously or intramuscularly. The duration of action is dependent upon the dose and route of administration of naloxone hydrochloride. Intramuscular administration produces a more prolonged effect than intravenous administration. The requirement for repeat doses of naloxone, however, will also be dependent upon the amount, type and route of administration of the narcotic being antagonized. Following parenteral administration naloxone hydrochloride is rapidly distributed in the body. It is metabolized in the liver, primarily by glucuronide conjugation, and excreted in urine. In one study the serum half-life in adults ranged from 30 to 81 minutes (mean 64±12 minutes). In a neonatal study the mean plasma half-life was observed to be 3.1±0.5 hours.

Provided herein are devices adapted for nasal delivery of a pharmaceutical composition, e.g., a pharmaceutical composition described herein, to a patient, comprising a therapeutically effective amount of an opioid antagonist selected from naloxone and pharmaceutically acceptable salts and hydrates thereof, wherein the therapeutically effective amount, is equivalent to about 2 mg to about 12 mg of naloxone hydrochloride. In some embodiments, the therapeutically effective amount is equivalent to about 2 mg to about 24 mg of naloxone hydrochloride. In some embodiments, the therapeutically effective amount is equivalent to about 2 mg to about 12 mg of naloxone hydrochloride. In some embodiments, the therapeutically effective amount is equivalent to about 3 mg to about 18 mg of naloxone hydrochloride. In some embodiments, the therapeutically effective amount is equivalent to about 4 mg to about 10 mg of naloxone hydrochloride. In some embodiments, the therapeutically effective amount is equivalent to about 5 mg to about 11 mg of naloxone hydrochloride. In some embodiments, the therapeutically effective amount is equivalent to about 6 mg to about 10 mg of naloxone hydrochloride. In some embodiments, the therapeutically effective amount is equivalent to about 4 mg to about 8 mg of naloxone hydrochloride. In some embodiments, the therapeutically effective amount is equivalent to about 7 mg to about 9 mg of naloxone hydrochloride. In some embodiments, the therapeutically effective amount is equivalent to about 3.4 mg of naloxone hydrochloride. In some embodiments, the therapeutically effective amount is equivalent to about 4 mg of naloxone hydrochloride. In some embodiments, the therapeutically effective amount is equivalent to about 5 mg of naloxone hydrochloride. In some embodiments, the therapeutically effective amount is equivalent to about 6 mg of naloxone hydrochloride. In some embodiments, the therapeutically effective amount is equivalent to about 7 mg of naloxone hydrochloride. In some embodiments, the therapeutically effective amount is equivalent to about 8 mg of naloxone hydrochloride. In some embodiments, the therapeutically effective amount is equivalent to about 9 mg of naloxone hydrochloride. In some embodiments, the therapeutically effective amount is equivalent to about 10 mg of naloxone hydrochloride. In some embodiments, the therapeutically effective amount is equivalent to about 11 mg of naloxone hydrochloride. In some embodiments, the therapeutically effective amount is equivalent to about 12 mg of naloxone hydrochloride. In some embodiments, the opioid antagonist is the only pharmaceutically active compound in pharmaceutical composition. In some embodiments, the opioid antagonist is naloxone hydrochloride. In some embodiments, the opioid antagonist is anhydrous naloxone hydrochloride. In some embodiments, the opioid antagonist is a hydrate of naloxone hydrochloride, e.g., naloxone hydrochloride dihydrate.

While many of the embodiments of the pharmaceutical compositions described herein will be described and exemplified with naloxone, other opioid antagonists can be adapted for nasal delivery based on the teachings of the specification. In fact, it should be readily apparent to one of ordinary skill in the art from the teachings herein that the devices and pharmaceutical compositions described herein may be suitable for other opioid antagonists. The opioid receptor antagonists described herein include μ-opioid antagonists and δ-opioid receptor antagonists. Examples of useful opioid receptor antagonists include naloxone, naltrexone, methylnaltrexone, and nalmefene. Other useful opioid receptor antagonists are known (see, e.g., Kreek et al., U.S. Pat. No. 4,987,136).

Also provided are devices adapted for nasal delivery of a pharmaceutical composition, e.g., a pharmaceutical composition described herein, to a patient, comprising a therapeutically effective amount of an opioid antagonist, wherein the therapeutically effective amount is about 4 mg to about 12 mg. In some embodiments, the therapeutically effective amount is equivalent to about 3.4 mg of naloxone hydrochloride. In some embodiments, the therapeutically effective amount is equivalent to about 4 mg of naloxone hydrochloride. In some embodiments, the therapeutically effective amount is equivalent to about 4 mg of naloxone hydrochloride. In some embodiments, the therapeutically effective amount is equivalent to about 6 mg of naloxone hydrochloride. In some embodiments, the therapeutically effective amount is equivalent to about 8 mg of naloxone hydrochloride. In some embodiments, the opioid antagonist is selected from naltrexone, methylnaltrexone, and nalmefene, and pharmaceutically acceptable salts and hydrates thereof. In some embodiments, the opioid antagonist is naltrexone hydrochloride. In some embodiments, the opioid antagonist is methylnaltrexone bromide. In some embodiments, the opioid antagonist is nalmefene hydrochloride. In some embodiments, the opioid antagonist is the only pharmaceutically active compound in pharmaceutical composition.

### Pharmaceutical Compositions

Also provided are pharmaceutical compositions comprising one or more opioid antagonist. In some embodiments the pharmaceutical compositions comprise an opioid antagonist and a pharmaceutically acceptable carrier. The carrier(s) must be “acceptable” in the sense of being compatible with the other ingredients of the formulation and not overly deleterious to the recipient thereof. Some embodiments of the present invention include a method of producing a pharmaceutical composition comprising admixing at least one opioid antagonist and a pharmaceutically acceptable carrier. Pharmaceutical compositions are applied directly to the nasal cavity using the devices described herein. In the case of a spray, this may be achieved for example by means of a metering atomizing spray pump.

Liquid preparations include solutions, suspensions and emulsions, for example, water or water-propylene glycol solutions. Additional ingredients in liquid preparations may include: antimicrobial preservatives, such as chlorobutanol, benzalkonium chloride, methylparaben, sodium benzoate, benzoic acid, phenyl ethyl alcohol, and the like, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof; a tonicity agent such as: dextrose, lactose, sodium chloride, calcium chloride, magnesium chloride, sorbitol, sucrose, mannitol, trehalose, raffinose, polyethylene glycol, hydroxyethyl starch, glycine, and the like, and mixtures thereof; and a suspending agent such as microcrystalline cellulose, carboxymethylcellulose sodium NF, polyacrylic acid, magnesium aluminum silicate, xanthan gum, and the like, and mixtures thereof.

The formulations of the present disclosure can also comprise one or more polyols, which include and are not limited to short-chain alkylene glycols (e.g., propylene glycol), glycerin, and polyethylene glycols.

The opioid antagonists described herein can be formulated into pharmaceutical compositions using techniques well known to those in the art. Suitable pharmaceutically acceptable carriers, outside those mentioned herein, are known in the art; for example, see Remington: The Science and Practice of Pharmacy, 21st ed., Lippincott Williams & Wilkins, Philadelphia, Pa. (2005).

The opioid antagonists described herein may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed by Berge et al., Journal of Pharmaceutical Sciences, 66:1-19 (1977). The acid addition salts may be obtained as the direct products of compound synthesis. In the alternative, the free base may be dissolved in a suitable solvent containing the appropriate acid and the salt isolated by evaporating the solvent or otherwise separating the salt and solvent. The opioid antagonists described herein may form solvates with standard low molecular weight solvents using methods known to the skilled artisan.

The pharmaceutical compositions described herein can comprise glycerin. For example, glycerin can be added to the compositions to limit the formation of small droplets (e.g., droplets with a diameter less than 10 microns) of the composition upon administration to a subject in need, increase the droplet residence time in the nasal mucosa or to prevent nasal dryness that could result from intranasal administration of the formulation. Glycerin can also be added to the pharmaceutical compositions to increase the solubility of the naloxone hydrochloride or hydrate thereof in the formulations described herein.

In one aspect, described herein is a pharmaceutical composition comprising naloxone hydrochloride or a hydrate thereof.

In one aspect, described herein is a pharmaceutical composition comprising naloxone hydrochloride or a hydrate thereof. In some embodiments, the pharmaceutical composition comprises a hydrate of naloxone hydrochloride, e.g., naloxone hydrochloride dihydrate.

In some embodiments, the pharmaceutical composition is an aqueous solution. In some embodiments, the pharmaceutical composition comprises chlorobutanol. In some embodiments, the pharmaceutical composition comprises a polyol less than 300 Da, e.g., propylene glycol or glycerin. In some embodiments, the pharmaceutical composition comprises glycerin. In some embodiments, the pharmaceutical composition comprises propylene glycol. In some embodiments, the pharmaceutical composition comprises an acid. In some embodiments, the acid is citric acid. In some embodiments, the pharmaceutical composition comprises a buffer. In some embodiments, the buffer comprises, e.g., consists essentially of or consists of, citric acid and trisodium citrate dihydrate. In some embodiments, the buffer is an acetate buffer. In some embodiments, the pharmaceutical composition comprises an isotonicity agent. In some embodiments, the isotonicity agent is sodium chloride. In some embodiments, the composition comprises a stabilizing agent. In some embodiments, the stabilizing agent is selected from the group consisting of EDTA and disodium ETDA.

In some embodiments, the volume of the aqueous solution is from about 50 μL to about 200 μL. In some embodiments, the volume of the aqueous solution is from about 80 μL to about 150 μL. In some embodiments, the volume of the aqueous solution is from about 90 μL to about 120 μL. In some embodiments, the volume of the aqueous solution is about 100 μL of the aqueous solution. In some embodiments, the volume of the aqueous solution is 133 μL of the aqueous solution.

In some embodiments, the concentration of naloxone hydrochloride or a hydrate thereof is greater than 16 mg per 100 μL of aqueous solution. In some embodiments, the concentration of naloxone hydrochloride or hydrate thereof is greater than 12 mg per 100 μL of the aqueous solution. In some embodiments, the concentration of naloxone hydrochloride or hydrate thereof is about 8 mg per 100 μL of the aqueous solution. In some embodiments, the concentration of naloxone hydrochloride or hydrate thereof is about 10 mg per 100 μL to about 16 mg per 100 μL of the aqueous solution. In some embodiments, the concentration of naloxone hydrochloride or hydrate thereof is about 10 mg per 100 μL of the aqueous solution. In some embodiments, the concentration of naloxone hydrochloride or hydrate thereof is about 12 mg per 100 μL of the aqueous solution. In some embodiments, the concentration of naloxone hydrochloride or hydrate thereof is from about 2 mg per 100 μL to about 16 mg per 100 μL of the aqueous solution. In some embodiments, the concentration of naloxone hydrochloride or hydrate thereof is from about 2 mg per 100 μL to about 12 mg per 100 μL of the aqueous solution. In some embodiments, the concentration of naloxone hydrochloride or hydrate thereof is from about 2 mg per 100 μL to about 10 mg per 100 μL of the aqueous solution. In some embodiments, the concentration of naloxone hydrochloride or hydrate thereof is from about 4 mg per 100 μL to about 8 mg per 100 μL of the aqueous solution. In some embodiments, the concentration of naloxone hydrochloride or hydrate thereof is selected from the group consisting of about 4 mg per 100 μL, about 6 mg per 100 μL, and about 8 mg per 133 μL of the aqueous solution. In some embodiments, the concentration of naloxone hydrochloride or hydrate thereof is about 4 mg per 100 μL of the aqueous solution. In some embodiments, the concentration of naloxone hydrochloride or hydrate thereof is about 4.4 mg per 100 μL of the aqueous solution. In some embodiments, the concentration of naloxone hydrochloride or hydrate thereof is about 6 mg per 100 μL of the aqueous solution. In some embodiments, the concentration of naloxone hydrochloride or hydrate thereof is about 8 mg per 133 μL of the aqueous solution.

In some embodiments, the hydrate of naloxone hydrochloride is naloxone hydrochloride dihydrate.

In some embodiments, the concentration of chlorobutanol is from about 0.1 mg per 100 μL to about 0.8 mg per 100 μL of the aqueous solution. In some embodiments, the concentration of chlorobutanol is from about 0.2 mg per 100 μL to about 0.6 mg per 100 μL of the aqueous solution. In some embodiments, the concentration of chlorobutanol is from about 0.3 mg per 100 μL to about 0.5 mg per 100 μL of the aqueous solution. In some embodiments, the concentration of chlorobutanol is from about 0.4 mg per 100 μL to about 0.5 mg per 100 μL of the aqueous solution. In some embodiments, the concentration of chlorobutanol is about 0.4 mg per 100 μL of the aqueous solution. In some embodiments, the concentration of chlorobutanol is about 0.45 mg per 100 μL of the aqueous solution. In some embodiments, the concentration of chlorobutanol is about 0.53 mg per 100 μL of the aqueous solution. In some embodiments, the concentration of the acid is from about 0.001 mg per 100 μL to about 0.15 mg per 100 μL of the aqueous solution. In some embodiments, the concentration of the acid is from about 0.001 mg per 100 μL to about 0.05 mg per 100 μL of the aqueous solution. In some embodiments, the concentration of the acid is from about 0.03 mg per 100 μL of the aqueous solution. In some embodiments, the concentration of the acid is from about 0.05 mg per 100 μL of the aqueous solution. In some embodiments, the concentration of the acid is from about 0.001 mg per 100 μL to about 0.01 mg per 100 μL of the aqueous solution. In some embodiments, the concentration of the acid is no more than about 0.15 mg per 100 μL of the aqueous solution. In some embodiments, the concentration of the acid is from about 0.001 mg per 100 μL to about 0.15 mg per 100 μL of the aqueous solution. In some embodiments, the concentration of the acid is from about 0.002 mg per 100 μL to about 0.009 mg per 100 μL of the aqueous solution. In some embodiments, the concentration of the acid is from about 0.004 mg per 100 μL to about 0.009 mg per 100 μL of the aqueous solution. In some embodiments, the concentration of the acid is from about 0.005 mg per 100 μL to about 0.009 mg per 100 μL of the aqueous solution. In some embodiments, the concentration of the acid is from about 0.006 mg per 100 μL to about 0.009 mg per 100 μL of the aqueous solution. In some embodiments, the concentration of the acid is from about 0.007 mg per 100 μL to about 0.008 mg per 100 μL of the aqueous solution. In some embodiments, the concentration of the acid is about 0.009 mg per 100 μL of the aqueous solution.

In some embodiments, the concentration of the buffer is no more than about 0.15 mg per 100 μL of the aqueous solution. In some embodiments, the concentration of the buffer is from about 0.001 mg per 100 μL to about 0.15 mg per 100 μL of the aqueous solution. In some embodiments, the concentration of the buffer is from about 0.002 mg per 100 μL to about 0.03 mg per 100 μL of the aqueous solution. In some embodiments, the concentration of the buffer is from about 0.002 mg per 100 μL to about 0.02 mg per 100 μL of the aqueous solution. In some embodiments, the concentration of the buffer is from about 0.004 mg per 100 μL to about 0.015 mg per 100 μL of the aqueous solution. In some embodiments, the concentration of the buffer is from about 0.008 mg per 100 μL to about 0.012 mg per 100 μL of the aqueous solution. In some embodiments, the concentration of the buffer is from about 0.009 mg per 100 μL to about 0.011 mg per 100 μL of the aqueous solution. In some embodiments, the concentration of the buffer is about 0.01 mg per 100 μL of the aqueous solution. In some embodiments, the concentration of the buffer is about 0.019 mg per 100 μL of the aqueous solution. In some embodiments, the concentration of the buffer is about 0.02 mg per 100 μL of the aqueous solution. In some embodiments, the concentration of the isotonicity agent is no more than about 2 mg per 100 μL of the aqueous solution. In some embodiments, the concentration of the isotonicity agent is from about 0.1 mg per 100 μL to about 2 mg per 100 μL of the aqueous solution. In some embodiments, the concentration of the isotonicity agent is from about 0.4 mg per 100 μL to about 1.5 mg per 100 μL of the aqueous solution. In some embodiments, the concentration of the isotonicity agent is from about 0.5 mg per 100 μL to about 1 mg per 100 μL of the aqueous solution. In some embodiments, the concentration of the isotonicity agent is from about 0.7 mg per 100 μL to about 0.9 mg per 100 μL of the aqueous solution. In some embodiments, the concentration of the isotonicity agent is from about 0.8 mg per 100 μL to about 0.9 mg per 100 μL of the aqueous solution. In some embodiments, the concentration of the isotonicity agent is about 0.85 mg per 100 μL of the aqueous solution. In some embodiments, the concentration of the isotonicity agent is about 0.625 mg per 100 μL of the aqueous solution. In some embodiments, the concentration of the glycerin is from about 0.1 mg per 100 μL to about 2 mg per 100 μL of the aqueous solution. In some embodiments, the concentration of the glycerin is from about 0.4 mg per 100 μL to about 1.8 mg per 100 μL of the aqueous solution. In some embodiments, the concentration of the glycerin is from about 0.8 mg per 100 μL to about 1.6 mg per 100 μL of the aqueous solution. In some embodiments, the concentration of the glycerin is from about 1 mg per 100 μL to about 1.6 mg per 100 μL of the aqueous solution. In some embodiments, the concentration of the glycerin is from about 1.3 mg per 100 μL to about 1.5 mg per 100 μL of the aqueous solution. In some embodiments, the concentration of the glycerin is about 1.4 mg per 100 μL of the aqueous solution.

In some embodiments, the osmolality of the composition is about 850 mOsm. In some embodiments, the osmolality of the composition is about 1000 mOsm. In some embodiments, the osmolality of the composition is from about 300 mOsm to about 700 mOsm. In some embodiments, the osmolality of the composition is from about 400 mOsm to about 700 mOsm. In some embodiments, the osmolality of the composition is from about 400 mOsm to about 650 mOsm. In some embodiments, the osmolality of the composition is about 446 mOsm. In some embodiments, the osmolality of the composition is about 614 mOsm. In some embodiments, the osmolality of the composition is about 613 mOsm. In some embodiments, the osmolality of the composition is about 607 mOsm. In some embodiments, the osmolality of the composition is about 446 mOsm and the composition does not comprise glycerin. In some embodiments, the osmolality of the composition is about 614 mOsm and the composition comprises glycerin. In some embodiments, the osmolality of the composition is about 607 mOsm and the composition comprises glycerin. In some embodiments, the osmolality of the composition is about 614 mOsm, the concentration of naloxone hydrochloride dihydrate is about 4 mg per 100 μL of aqueous solution, and the composition comprises glycerin. In some embodiments, the osmolality of the composition is about 613 mOsm, the concentration of naloxone hydrochloride dihydrate is about 4 mg per 100 μL of aqueous solution, and the composition comprises glycerin. In some embodiments, the osmolality of the composition is about 607 mOsm, the concentration of naloxone hydrochloride dihydrate is about 6 mg per 100 μL of aqueous solution, and the composition comprises glycerin. In some embodiments, the osmolality of the composition is about 607 mOsm, the concentration of naloxone hydrochloride dihydrate is about 8 mg per 133 μL of aqueous solution, and the composition comprises glycerin.

In some embodiments, the pH of the composition is from about 3 to about 6. In some embodiments, the pH of the composition is from about 4 to about 5. In some embodiments, the pH of the composition is from about 3.5 to about 4.7. In some embodiments, the pH of the composition is about 4.1. In some embodiments, the pH of the composition is about 4.0. In some embodiments, the composition is formulated for intranasal administration. In some embodiments, when intranasally administered to a subject, the pH of the composition is the pH of the nasal mucosa of the subject. In some embodiments, when intranasally administered to a subject, the pharmaceutical composition yields a naloxone Tₘₐₓ of less than 30 minutes. In some embodiments, when intranasally administered to a subject, the pharmaceutical composition yields a naloxone Tₘₐₓ of less than 25 minutes. In some embodiments, when intranasally administered to a subject, the pharmaceutical composition yields a naloxone Tₘₐₓ of less than 20 minutes. In some embodiments, when intranasally administered to a subject, the pharmaceutical composition yields a mean naloxone plasma concentration of ≥0.2 ng/mL within 2.5 minutes in said subject.

In some embodiments, when intranasally administered to a subject, the pharmaceutical composition yields a mean naloxone plasma concentration of ≥1 ng/mL within 5 minutes in said subject. In some embodiments, when intranasally administered to a subject, the pharmaceutical composition yields a mean naloxone plasma concentration of ≥3 ng/mL within 10 minutes in said subject.

In some embodiments, upon nasal delivery of said pharmaceutical composition to said patient, less than about 20% of said pharmaceutical composition leaves the nasal cavity via drainage into the nasopharynx or externally. In some embodiments, upon nasal delivery of said pharmaceutical composition to said patient, less than about 10% of said pharmaceutical composition leaves the nasal cavity via drainage into the nasopharynx or externally. In some embodiments, upon nasal delivery of said pharmaceutical composition to said patient, less than about 5% of said pharmaceutical composition leaves the nasal cavity via drainage into the nasopharynx or externally.

In some embodiments, the plasma concentration versus time curve of said opioid antagonist in a patient has a Tₘₐₓ of less than 30 minutes. In some embodiments, the plasma concentration versus time curve of said opioid antagonist in said patient has a Tₘₐₓ of less than 25 minutes. In some embodiments, the plasma concentration versus time curve of said opioid antagonist in said patient has a Tₘₐₓ of about 20 minutes. In some embodiments, delivery of said pharmaceutical formulation to a patient, provides occupancy at Tₘₐₓ of said opioid antagonist at the opioid receptors in the respiratory control center of said patient of greater than about 90%. In some embodiments, delivery of said pharmaceutical formulation to said patient, provides occupancy at Tₘₐₓ of said opioid antagonist at the opioid receptors in the respiratory control center of said patient of greater than about 95%. In some embodiments, delivery of said pharmaceutical formulation to said patient, provides occupancy at Tₘₐₓ of said opioid antagonist at the opioid receptors in the respiratory control center of said patient of greater than about 99%.

In some embodiments, said patient is free from respiratory depression for at least about 1 hour following treatment comprising essentially of delivery of said therapeutically effective amount of said opioid antagonist. In some embodiments, said patient is free from respiratory depression for at least about 2 hours following treatment comprising essentially of delivery of said therapeutically effective amount of said opioid antagonist. In some embodiments, said patient is free from respiratory depression for at least about 4 hours following treatment comprising essentially of delivery of said therapeutically effective amount of said opioid antagonist. In some embodiments, said patient is free from respiratory depression for at least about 6 hours following treatment comprising essentially of delivery of said therapeutically effective amount of said opioid antagonist.

In one aspect, described herein is a pharmaceutical composition comprising naloxone hydrochloride or a hydrate thereof, chlorobutanol, citric acid, trisodium citrate dihydrate, sodium chloride, and glycerin.

In some embodiments, the hydrate of naloxone hydrochloride is naloxone hydrochloride dihydrate. In some embodiments, the concentration of naloxone hydrochloride dihydrate is selected from the group consisting of about 4 mg per 100 μL of composition, about 6 mg per 100 μL of composition, and about 8 mg per 133 μL of composition the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is selected from the group consisting of about 0.85 mg per 100 μL of composition and about 0.625 mg per 100 μL of composition, and the concentration of glycerin is about 1.4 mg per 100 μL of composition. In some embodiments, the concentration of naloxone hydrochloride dihydrate is about 4 mg per 100 μL of composition, the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is about 0.85 mg per 100 μL of composition, and the concentration of glycerin is about 1.4 mg per 100 μL of composition. In some embodiments, the concentration of naloxone hydrochloride dihydrate is selected from the group consisting of about 6 mg per 100 μL of composition, the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is selected from the group consisting of about 0.625 mg per 100 μL of composition, and the concentration of glycerin is about 1.4 mg per 100 μL of composition. In some embodiments, the concentration of naloxone hydrochloride dihydrate is selected from the group consisting of about 8 mg per 133 μL of composition the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is selected from the group consisting of about 0.625 mg per 100 μL of composition, and the concentration of glycerin is about 1.4 mg per 100 μL of composition.

In some embodiments, the pharmaceutical composition is an aqueous solution. In some embodiments, the volume of the aqueous solution is from about 50 μL to about 200 μL. In some embodiments, the volume of the aqueous solution is from about 80 μL to about 150 μL. In some embodiments, the volume of the aqueous solution is from about 90 μL to about 120 μL. In some embodiments, the volume of the aqueous solution is about 100 μL of the aqueous solution. In some embodiments, the volume of the aqueous solution is about 133 μL of the aqueous solution.

In some embodiments, the osmolality of the composition is from about 300 mOsm to about 700 mOsm. In some embodiments, the osmolality of the composition is from about 400 mOsm to about 700 mOsm. In some embodiments, the osmolality of the composition is from about 400 mOsm to about 650 mOsm. In some embodiments, the osmolality of the composition is about 614 mOsm. In some embodiments, the osmolality of the composition is about 613 mOsm. In some embodiments, the osmolality of the composition is about 607 mOsm. In some embodiments, the osmolality of the composition is about 614 mOsm and the concentration of naloxone hydrochloride dihydrate is about 4 mg per 100 μL. In some embodiments, the osmolality of the composition is about 613 mOsm and the concentration of naloxone hydrochloride dihydrate is about 4 mg per 100 μL. In some embodiments, the osmolality of the composition is about 607 mOsm and the concentration of naloxone hydrochloride dihydrate is about 6 mg per 100 μL. In some embodiments, the osmolality of the composition is about 607 mOsm and the concentration of naloxone hydrochloride dihydrate is about 8 mg per 133 μL.

In some embodiments, the pH of the composition is from about 3 to about 6. In some embodiments, the pH of the composition is from about 4 to about 5. In some embodiments, the pH of the composition is about 4.3. In some embodiments, the pH of the composition is about 4.1. In some embodiments, the composition is formulated for intranasal administration. In some embodiments, the pH of the composition is the pH of the nasal mucosa of the subject when intranasally administered to a subject.

In some embodiments, the pharmaceutical composition yields, when intranasally administered to a subject, a naloxone Tₘₐₓ of less than 30 minutes. In some embodiments, the pharmaceutical composition yields, when intranasally administered to a subject, a naloxone Tₘₐₓ of less than 25 minutes. In some embodiments, the pharmaceutical composition yields, when intranasally administered to a subject, a naloxone Tₘₐₓ of less than 20 minutes. In some embodiments, the pharmaceutical composition yields, when intranasally administered to a subject, a mean naloxone plasma concentration of ≥0.2 ng/mL within 2.5 minutes in said subject. In some embodiments, the pharmaceutical composition yields, when intranasally administered to a subject, a mean naloxone plasma concentration of ≥1 ng/mL within 5 minutes in said subject. In some embodiments, the pharmaceutical composition yields, when intranasally administered to a subject, a mean naloxone plasma concentration of ≥3 ng/mL within 10 minutes in said subject.

In some embodiments, upon nasal delivery of said pharmaceutical composition to said patient, less than about 20% of said pharmaceutical composition leaves the nasal cavity via drainage into the nasopharynx or externally. In some embodiments, upon nasal delivery of said pharmaceutical composition to said patient, less than about 10% of said pharmaceutical composition leaves the nasal cavity via drainage into the nasopharynx or externally. In some embodiments, upon nasal delivery of said pharmaceutical composition to said patient, less than about 5% of said pharmaceutical composition leaves the nasal cavity via drainage into the nasopharynx or externally.

In some embodiments, the plasma concentration versus time curve of said opioid antagonist in a patient has a Tₘₐₓ of less than 30 minutes. In some embodiments, the plasma concentration versus time curve of said opioid antagonist in said patient has a Tₘₐₓ of less than 25 minutes. In some embodiments, the plasma concentration versus time curve of said opioid antagonist in said patient has a Tₘₐₓ of about 20 minutes. In some embodiments, the plasma concentration versus time curve of the opioid antagonist in the patient has a Tₘₐₓ of less than 19 minutes. In some embodiments, the plasma concentration versus time curve of the opioid antagonist in the patient has a Tₘₐₓ of about 18.5 minutes.

In some embodiments, delivery of said pharmaceutical formulation to a patient, provides occupancy at Tₘₐₓ of said opioid antagonist at the opioid receptors in the respiratory control center of said patient of greater than about 90%. In some embodiments, delivery of said pharmaceutical formulation to said patient, provides occupancy at Tₘₐₓ of said opioid antagonist at the opioid receptors in the respiratory control center of said patient of greater than about 95%. In some embodiments, delivery of said pharmaceutical formulation to said patient, provides occupancy at Tₘₐₓ of said opioid antagonist at the opioid receptors in the respiratory control center of said patient of greater than about 99%. In some embodiments, said patient is free from respiratory depression for at least about 1 hour following treatment comprising essentially of delivery of said therapeutically effective amount of said opioid antagonist. In some embodiments, said patient is free from respiratory depression for at least about 2 hours following treatment comprising essentially of delivery of said therapeutically effective amount of said opioid antagonist. In some embodiments, said patient is free from respiratory depression for at least about 4 hours following treatment comprising essentially of delivery of said therapeutically effective amount of said opioid antagonist. In some embodiments, said patient is free from respiratory depression for at least about 6 hours following treatment comprising essentially of delivery of said therapeutically effective amount of said opioid antagonist.

In one aspect, provided herein is a pharmaceutical composition comprising naloxone hydrochloride or a hydrate thereof, morphine, hydrocodone, hydromorphone, oxycodone, oxymorphone, buprenorphine, levonaloxone, pseudomorphine, nalbutene, codeine, dextromethorophan, fentanyl, methadone, tramadol and other opioid antagonists; cyprodime, nalmefene, nalodeine, naloxol, naltrexone, or nalbuphene, and glycerin.

In some embodiments, the pharmaceutical composition comprises naloxone hydrochloride or a hydrate thereof and glycerin. In some embodiments, the pharmaceutical composition is an aqueous solution. In some embodiments, the concentration of glycerin is from about 0.5 mg per 100 μL of aqueous solution to about 2.5 mg per 100 μL of aqueous solution. In some embodiments, the concentration of glycerin is from about 2.5 mg per 100 μL of aqueous solution to about 5 mg per 100 μL of the aqueous solution. In some embodiments, the concentration of the glycerin is from about 0.1 mg per 100 μL to about 2 mg per 100 μL of the aqueous solution. In some embodiments, the concentration of the glycerin is from about 0.4 mg per 100 μL to about 1.8 mg per 100 μL of the aqueous solution. In some embodiments, the concentration of the glycerin is from about 0.8 mg per 100 μL to about 1.6 mg per 100 μL of the aqueous solution. In some embodiments, the concentration of the glycerin is from about 1 mg per 100 μL to about 1.6 mg per 100 μL of the aqueous solution. In some embodiments, the concentration of the glycerin is from about 1.3 mg per 100 μL to about 1.5 mg per 100 μL of the aqueous solution. In some embodiments, the concentration of glycerin is about 2.5 mg per 100 μL of aqueous solution. In some embodiments, the concentration of glycerin is about 1.4 mg per 100 μL of aqueous solution.

In some embodiments, the volume of the aqueous solution is from about 50 μL to about 200 μL. In some embodiments, the volume of the aqueous solution is from about 80 μL to about 150 μL. In some embodiments, the volume of the aqueous solution is from about 90 μL to about 120 μL. In some embodiments, the volume of the aqueous solution is about 100 μL of the aqueous solution. In some embodiments, the volume of the aqueous solution is 133 μL of the aqueous solution.

In some embodiments, the concentration of naloxone hydrochloride or hydrate thereof is greater than 16 mg per 100 μL of the aqueous solution. In some embodiments, the concentration of naloxone hydrochloride or a hydrate thereof is greater than 12 mg per 100 μL of aqueous solution. In some embodiments, the concentration of naloxone hydrochloride or a hydrate thereof is about 12 mg per 100 μL of aqueous solution. In some embodiments, the concentration of naloxone hydrochloride or a hydrate thereof is from about 2 mg per 100 μL of aqueous solution to about 16 mg per 100 μL of aqueous solution. In some embodiments, the concentration of naloxone hydrochloride or a hydrate thereof is from about 1 mg per 100 μL of aqueous solution to about 12 mg per 100 μL of aqueous solution. In some embodiments, the concentration of naloxone hydrochloride or hydrate thereof is from about 2 mg per 100 μL to about 10 mg per 100 μL of the aqueous solution. In some embodiments, the concentration of naloxone hydrochloride or hydrate thereof is from about 4 mg per 100 μL to about 8 mg per 100 μL of the aqueous solution. In some embodiments, the concentration of naloxone hydrochloride or hydrate thereof is selected from the group consisting of about 4 mg per 100 μL, about 6 mg per 100 μL, and about 8 mg per 133 μL of the aqueous solution of the aqueous solution. In some embodiments, the concentration of naloxone hydrochloride or hydrate thereof is about 4 mg per 100 μL of the aqueous solution. In some embodiments, the concentration of naloxone hydrochloride or hydrate thereof is about 4.4 mg per 100 μL of the aqueous solution. In some embodiments, the concentration of naloxone hydrochloride or hydrate thereof is about 6 mg per 100 μL of the aqueous solution. In some embodiments, the concentration of naloxone hydrochloride or hydrate thereof is about 8 mg per 133 μL of the aqueous solution.

In some embodiments, the hydrate of naloxone hydrochloride is naloxone hydrochloride dihydrate.

In some embodiments, the concentration of naloxone hydrochloride or hydrate thereof is from about 8 mg per 100 μL to about 16 mg per 100 μL of aqueous solution and the concentration of glycerin is about 1.4 mg 100 μL of aqueous solution. In some embodiments, the concentration of naloxone hydrochloride or hydrate thereof is from about 8 mg per 100 μL to about 16 mg per 100 μL of aqueous solution and the concentration of glycerin is about 2.5 mg 100 μL of aqueous solution.

In some embodiments, the concentration of naloxone hydrochloride or hydrate thereof is from about 10 mg per 100 μL to about 16 mg per 100 μL of aqueous solution and the concentration of glycerin is about 1.4 mg 100 μL of aqueous solution. In some embodiments, the concentration of naloxone hydrochloride or hydrate thereof is from about 10 mg per 100 μL to about 16 mg per 100 μL of aqueous solution and the concentration of glycerin is about 2.5 mg 100 μL of aqueous solution.

In some embodiments, the concentration of naloxone hydrochloride or hydrate thereof is about 12 mg per 100 μL of aqueous solution and the concentration of glycerin is about 1.4 mg 100 μL of aqueous solution. In some embodiments, the concentration of naloxone hydrochloride or hydrate thereof is about 12 mg per 100 μL of aqueous solution and the concentration of glycerin is about 2.5 mg 100 μL of aqueous solution.

In some embodiments, the pharmaceutical composition comprises chlorobutanol. In some embodiments, the concentration of chlorobutanol is from about 0.1 mg per 100 μL to about 0.8 mg per 100 μL of the aqueous solution. In some embodiments, the concentration of chlorobutanol is from about 0.2 mg per 100 μL to about 0.6 mg per 100 μL of the aqueous solution. In some embodiments, the concentration of chlorobutanol is from about 0.3 mg per 100 μL to about 0.5 mg per 100 μL of the aqueous solution. In some embodiments, the concentration of chlorobutanol is from about 0.4 mg per 100 μL to about 0.5 mg per 100 μL of the aqueous solution. In some embodiments, the concentration of chlorobutanol is about 0.4 mg per 100 μL of the aqueous solution. In some embodiments, the concentration of chlorobutanol is about 0.45 mg per 100 μL of the aqueous solution. In some embodiments, the concentration of chlorobutanol is about 0.53 mg per 100 μL of the aqueous solution. In some embodiments, the concentration of the acid is from about 0.001 mg per 100 μL to about 0.15 mg per 100 μL of the aqueous solution. In some embodiments, the concentration of the acid is from about 0.001 mg per 100 μL to about 0.05 mg per 100 μL of the aqueous solution. In some embodiments, the concentration of the acid is from about 0.001 mg per 100 μL to about 0.03 mg per 100 μL of the aqueous solution. In some embodiments, the concentration of the acid is from about 0.03 mg per 100 μL of the aqueous solution. In some embodiments, the concentration of the acid is from about 0.05 mg per 100 μL of the aqueous solution. In some embodiments, the concentration of the acid is no more than about 0.15 mg per 100 μL of the aqueous solution. In some embodiments, the concentration of the acid is from about 0.001 mg per 100 μL to about 0.15 mg per 100 μL of the aqueous solution. In some embodiments, the concentration of the acid is from about 0.002 mg per 100 μL to about 0.009 mg per 100 μL of the aqueous solution. In some embodiments, the concentration of the acid is from about 0.004 mg per 100 μL to about 0.009 mg per 100 μL of the aqueous solution. In some embodiments, the concentration of the acid is from about 0.005 mg per 100 μL to about 0.009 mg per 100 μL of the aqueous solution. In some embodiments, the concentration of the acid is from about 0.006 mg per 100 μL to about 0.009 mg per 100 μL of the aqueous solution. In some embodiments, the concentration of the acid is from about 0.006 mg per 100 μL to about 0.008 mg per 100 μL of the aqueous solution. In some embodiments, the concentration of the acid is about 0.009 mg per 100 μL of the aqueous solution.

In some embodiments, the pharmaceutical composition comprises a buffer. In some embodiments, the buffer comprises, e.g., consists essentially of or consists of, citric acid and trisodium citrate. In some embodiments, the buffer comprises, e.g., consists essentially of or consists of, an acetate buffer. In some embodiments, the concentration of the buffer is no more than about 0.15 mg per 100 μL of the aqueous solution. In some embodiments, the concentration of the buffer is from about 0.001 mg per 100 μL to about 0.15 mg per 100 μL of the aqueous solution. In some embodiments, the concentration of the buffer is from about 0.002 mg per 100 μL to about 0.03 mg per 100 μL of the aqueous solution. In some embodiments, the concentration of the buffer is from about 0.002 mg per 100 μL to about 0.02 mg per 100 μL of the aqueous solution. In some embodiments, the concentration of the buffer is from about 0.004 mg per 100 μL to about 0.015 mg per 100 μL of the aqueous solution. In some embodiments, the concentration of the buffer is from about 0.008 mg per 100 μL to about 0.012 mg per 100 μL of the aqueous solution. In some embodiments, the concentration of the buffer is from about 0.009 mg per 100 μL to about 0.011 mg per 100 μL of the aqueous solution. In some embodiments, the concentration of the buffer is about 0.01 mg per 100 μL of the aqueous solution. In some embodiments, the concentration of the buffer is about 0.019 mg per 100 μL of the aqueous solution. In some embodiments, the concentration of the buffer is about 0.02 mg per 100 μL of the aqueous solution.

In some embodiments, the pharmaceutical composition comprises an isotonicity agent. In some embodiments, the isotonicity agent is sodium chloride. In some embodiments, the concentration of the isotonicity agent is no more than about 2 mg per 100 μL of the aqueous solution. In some embodiments, the concentration of the isotonicity agent is from about 0.1 mg per 100 μL to about 2 mg per 100 μL of the aqueous solution. In some embodiments, the concentration of the isotonicity agent is from about 0.4 mg per 100 μL to about 1.5 mg per 100 μL of the aqueous solution. In some embodiments, the concentration of the isotonicity agent is from about 0.5 mg per 100 μL to about 1 mg per 100 μL of the aqueous solution. In some embodiments, the concentration of the isotonicity agent is from about 0.7 mg per 100 μL to about 0.9 mg per 100 μL of the aqueous solution. In some embodiments, the concentration of the isotonicity agent is from about 0.8 mg per 100 μL to about 0.9 mg per 100 μL of the aqueous solution. In some embodiments, the concentration of the isotonicity agent is about 0.85 mg per 100 μL of the aqueous solution. In some embodiments, the concentration of the isotonicity agent is about 0.625 mg per 100 μL of the aqueous solution.

In some embodiments, the osmolality of the composition is about 850 mOsm. In some embodiments, the osmolality of the composition is about 1000 mOsm. In some embodiments, the osmolality of the composition is from about 300 mOsm to about 700 mOsm. In some embodiments, the osmolality of the composition is from about 400 mOsm to about 700 mOsm. In some embodiments, the osmolality of the composition is from about 400 mOsm to about 650 mOsm. In some embodiments, the osmolality of the composition is about 446 mOsm. In some embodiments, the osmolality of the composition is about 614 mOsm. In some embodiments, the osmolality of the composition is about 613 mOsm. In some embodiments, the osmolality of the composition is about 607 mOsm. In some embodiments, the osmolality of the composition is about 446 mOsm and the composition does not comprise glycerin. In some embodiments, the osmolality of the composition is about 614 mOsm and the composition comprises glycerin. In some embodiments, the osmolality of the composition is about 607 mOsm and the composition comprises glycerin. In some embodiments, the osmolality of the composition is about 614 mOsm, the concentration of naloxone hydrochloride dihydrate is about 4 mg per 100 μL of aqueous solution, and the composition comprises glycerin. In some embodiments, the osmolality of the composition is about 613 mOsm, the concentration of naloxone hydrochloride dihydrate is about 4 mg per 100 μL of aqueous solution, and the composition comprises glycerin. In some embodiments, the osmolality of the composition is about 607 mOsm, the concentration of naloxone hydrochloride dihydrate is about 6 mg per 100 μL of aqueous solution, and the composition comprises glycerin. In some embodiments, the osmolality of the composition is about 607 mOsm, the concentration of naloxone hydrochloride dihydrate is about 8 mg per 133 μL of aqueous solution, and the composition comprises glycerin.

In some embodiments, the pH of the composition is from about 3 to about 6. In some embodiments, the pH of the composition is from about 4 to about 5. In some embodiments, the pH of the composition is about 4.1. In some embodiments, the pH of the composition is about 4.0. In some embodiments, the composition is formulated for intranasal administration. In some embodiments, when intranasally administered to a subject, the pH of the composition is the pH of the nasal mucosa of the subject. In some embodiments, when intranasally administered to a subject, the pharmaceutical composition yields a naloxone Tₘₐₓ of less than 30 minutes. In some embodiments, when intranasally administered to a subject, the pharmaceutical composition yields a naloxone Tₘₐₓ of less than 25 minutes. In some embodiments, when intranasally administered to a subject, the pharmaceutical composition yields a naloxone Tₘₐₓ of less than 20 minutes. In some embodiments, when intranasally administered to a subject, the pharmaceutical composition yields a mean naloxone plasma concentration of ≥0.2 ng/mL within 2.5 minutes in said subject. In some embodiments, when intranasally administered to a subject, the pharmaceutical composition yields a mean naloxone plasma concentration of ≥1 ng/mL within 5 minutes in said subject. In some embodiments, when intranasally administered to a subject, the pharmaceutical composition yields a mean naloxone plasma concentration of ≥3 ng/mL within 10 minutes in said subject.

Also provided are devices for “combination-therapy” comprising pharmaceutical compositions comprising at least one opioid antagonist described herein, together with at least one known pharmaceutical agent and a pharmaceutically acceptable carrier. In some embodiments, the pharmaceutical composition comprises a short-acting opioid antagonist and a long-acting opioid antagonist. In some embodiments, the pharmaceutical composition comprises naloxone and naltrexone. In some embodiments, the pharmaceutical composition comprises naloxone and methylnaltrexone. In some embodiments, the pharmaceutical composition comprises naloxone and nalmefene.

In one aspect, provided herein is a pharmaceutical composition comprising naloxone hydrochloride or a hydrate thereof, e.g., naloxone hydrochloride dihydrate, chlorobutanol, trisodium citrate dihydrate, and glycerin. In some embodiments the pharmaceutical composition is an aqueous solution. In some embodiments, the volume of the aqueous solution is about 100 μL. In some embodiments, the pharmaceutical composition comprises about 13.2 mg of naloxone hydrochloride dihydrate per 100 μL of aqueous solution, about 0.53 mg of chlorobutanol hemihydrate per 100 μL of aqueous solution, about 0.009 mg of anhydrous citric acid per 100 μL of aqueous solution about 0.015 mg of trisodium citrate dihydrate per 100 μL of aqueous solution, and about 1.4 mg of glycerin per 100 μL of aqueous solution. In some embodiments, the pH of the aqueous solution is from about 3.5 to about 4.7. In some embodiments, the pH of the aqueous solution is about 4.1. In some embodiments, the osmolality of the pharmaceutical composition is from about 400 mOsm to about 850 mOsm. In some embodiments, the osmolality of the pharmaceutical composition is about 615 mOsm. In some embodiments the density of the aqueous solution is about 1.04 mg/mL.

In some embodiments, the composition is formulated for intranasal administration. In some embodiments, when intranasally administered to a subject, the pH of the composition is the pH of the nasal mucosa of the subject. In some embodiments, when intranasally administered to a subject, the pharmaceutical composition yields a naloxone Tₘₐₓ of less than 30 minutes. In some embodiments, when intranasally administered to a subject, the pharmaceutical composition yields a naloxone Tₘₐₓ of less than 25 minutes. In some embodiments, when intranasally administered to a subject, the pharmaceutical composition yields a naloxone Tₘₐₓ of less than 20 minutes. In some embodiments, when intranasally administered to a subject, the pharmaceutical composition yields a mean naloxone plasma concentration of ≥0.2 ng/mL within 2.5 minutes in said subject. In some embodiments, when intranasally administered to a subject, the pharmaceutical composition yields a mean naloxone plasma concentration of ≥1 ng/mL within 5 minutes in said subject. In some embodiments, when intranasally administered to a subject, the pharmaceutical composition yields a mean naloxone plasma concentration of ≥3 ng/mL within 10 minutes in said subject.

### Nasal Drug Delivery Devices and Kits

Also provided are nasal drug delivery devices comprising a pharmaceutical composition described herein. Nasal delivery is considered an attractive route for needle-free, systemic drug delivery, especially when rapid absorption and effect are desired. In addition, nasal delivery may help address issues related to poor bioavailability, slow absorption, drug degradation, and adverse events (AEs) in the gastrointestinal tract and avoids the first-pass metabolism in the liver.

Liquid nasal formulations are mainly aqueous solutions, but suspensions and emulsions can also be delivered. In traditional spray pump systems, antimicrobial preservatives are typically required to maintain microbiological stability in liquid formulations.

Some EMS programs have developed a system using existing technologies of an approved drug and an existing medical device to administer naloxone intranasally, albeit in a non-FDA approved manner. This has been accomplished by using the injectable formulation (1 mg/mL) and administering 1 mL per nostril via a marketed nasal atomizer/nebulizer device. The system combines an FDA-approved naloxone injection product (with a Luer fitted tip, no needles) with a marketed, medical device called the Mucosal Atomization Device (MAD™ Nasal, Wolfe Tory Medical, Inc.). This initiative is consistent with the U.S. Needlestick Safety and Prevention Act (Public Law 106-430). The EMS programs recognize limitations of this system, one limitation being that it is not assembled and ready-to-use. Although this administration mode appears to be effective in reversing narcosis, the formulation is not concentrated for retention in the nasal cavity. The 1 mL delivery volume per nostril is larger than that generally utilized for intranasal drug administration. Therefore, there is loss of drug from the nasal cavity, due either to drainage into the nasopharynx or externally from the nasal cavity. The devices described herein are improved ready-to-use products specifically optimized, concentrated, and formulated for nasal delivery.

Metered spray pumps have dominated the nasal drug delivery market since they were introduced. The pumps typically deliver 100 μL (25-200 μL) per spray, and they offer high reproducibility of the emitted dose and plume geometry in in vitro tests. The particle size and plume geometry can vary within certain limits and depend on the properties of the pump, the formulation, the orifice of the actuator, and the force applied. Traditional spray pumps replace the emitted liquid with air, and preservatives are therefore required to prevent contamination. However, driven by the studies suggesting possible negative effects of preservatives, pump manufacturers have developed different spray systems that avoid the need for preservatives. These systems use a collapsible bag, a movable piston, or a compressed gas to compensate for the emitted liquid volume (www.aptar.com and www.rexam.com). The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a headdown application is recommended. Another method used for avoiding preservatives is that the air that replaces the emitted liquid is filtered through an aseptic air filter. In addition, some systems have a ball valve at the tip to prevent contamination of the liquid inside the applicator tip (www.aptar.com). More recently, pumps have been designed with side-actuation and introduced for delivery of fluticasone furoate for the indication of seasonal and perennial allergic rhinitis. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available (www.rexam.com and www.aptar.com).

Metered-dose spray pumps require priming and some degree of overfill to maintain dose conformity for the labeled number of doses. They are well suited for drugs to be administered daily over a prolonged duration, but due to the priming procedure and limited control of dosing, they are less suited for drugs with a narrow therapeutic window. For expensive drugs and vaccines intended for single administration or sporadic use and where tight control of the dose and formulation is of particular importance, single-dose or bi-dose spray devices are preferred (www.aptar.com). A simple variant of a single-dose spray device (MAD™) is offered by LMA (LMA, Salt Lake City, Utah, USA; www.mana.com). A nosepiece with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (ACCUSPRAY™, Becton Dickinson Technologies, Research Triangle Park, N.C., USA; www.bdphanna.com) is used to deliver the influenza vaccine FluMist (www.flumist.com), approved for both adults and children in the US market. A similar device for two doses was marketed by a Swiss company for delivery of another influenza vaccine a decade ago. The single- and bi-dose devices mentioned above consist of a reservoir, a piston, and a swirl chamber (see, e.g., the UDS UnitDose and BDS BiDose devices from Aptar, formerly Pfeiffer). The spray is formed when the liquid is forced out through the swirl chamber. These devices are held between the second and the third fingers with the thumb on the actuator. A pressure point mechanism incorporated in some devices secures reproducibility of the actuation force and emitted plume characteristics. Currently, marketed nasal migraine drugs like Imitrex (www.gsk.com) and Zomig (www.az.com; Pfeiffer/Aptar single-dose device) and the marketed influenza vaccine Flu-Mist (www.flumist.com; Becton Dickinson single-dose spray device) are delivered with this type of device.

With sterile filling, the use of preservatives is not required, but overfill is required resulting in a waste fraction similar to the metered-dose, multi-dose sprays. To emit 100 μL, a volume of 125 μL is filled in the device (Pfeiffer/Aptar single-dose device) used for the intranasal migraine medications Imitrex (sumatriptan) and Zomig (zolmitriptan) and about half of that for a bi-dose design. Sterile drug products may be produced using aseptic processing or terminal sterilization. Terminal sterilization usually involves filling and sealing product containers under high-quality environmental conditions. Products are filled and sealed in this type of environment to minimize the microbial and particulate content of the in-process product and to help ensure that the subsequent sterilization process is successful. In most cases, the product, container, and closure have low bioburden, but they are not sterile. The product in its final container is then subjected to a sterilization process such as heat or irradiation. In an aseptic process, the drug product, container, and closure are first subjected to sterilization methods separately, as appropriate, and then brought together. Because there is no process to sterilize the product in its final container, it is critical that containers be filled and sealed in an extremely high-quality environment. Aseptic processing involves more variables than terminal sterilization. Before aseptic assembly into a final product, the individual parts of the final product are generally subjected to various sterilization processes. For example, glass containers are subjected to dry heat; rubber closures are subjected to moist heat; and liquid dosage forms are subjected to filtration. Each of these manufacturing processes requires validation and control.

The droplet size distribution of a nasal spray is a critical parameter, since it significantly influences the in vivo deposition of the drug in the nasal cavity. The droplet size is influenced by the actuation parameters of the device and the formulation. The prevalent median droplet size should be between about 30 and about 100 μm. If the droplets are too large (e.g., greater than about 120 μm), deposition takes place mainly in the anterior parts of the nose, and if the droplets are too small (e.g., less than about 10 μm), they can possibly be inhaled and reach the lungs, which should be avoided because of safety reasons.

Spray characterization (e.g., plume geometry, spray pattern, pump delivery, droplet size distribution (DSD)) of the delivered plume subsequent to spraying may be measured under specified experimental and instrumental conditions by appropriate and validated and/or calibrated analytical procedures known in the art. These include photography, laser diffraction, and impaction systems (cascade impaction, next generation impaction (NGI), etc.). Droplet size distribution can be controlled in terms of ranges for the D10, D50, D90, span [(D90-D10)/050], and percentage of droplets less than 10 mm. In certain embodiments, the formulation will have a narrow DSD. The particle diameter “(D)” designations refer to the representative diameter where 10% (D10), 50% (D50) and 90% (D90) of the total volume of the liquid sprayed is made up of droplets with diameters smaller than or equal to the stated value.

In certain embodiments, the percent of droplets less than 10 μm is less than 12%. In certain embodiments, the percent of droplets less than 10 μm is less than 10%. In certain embodiments, the percent of droplets less than 10 μm is less than 5%. In certain embodiments, the percent of droplets less than 10 μm is less than 2%. In certain embodiments, the percent of droplets less than 10 μm is less than 1%.

In one aspect, described herein is a device configured for intranasally administering of a pharmaceutical composition described herein, wherein the device is configured for delivery of one dose of the pharmaceutical composition to the subject.

In some embodiments, the dose is contained in a single reservoir. In some embodiments, the device is adapted for single use. In some embodiments, the device is configured for delivery of the dose to the subject by a single actuation. In some embodiments, the device is not primed prior administering the dose to the subject.

In some embodiments, the volume of said reservoir is not more than about 200 μL. In some embodiments, the volume of said reservoir is not more than about 140 μL. In some embodiments, the volume of the single dose in the reservoir is from about 90 μL to about 140 μL. In some embodiments, the volume of the single dose in the reservoir is from about 95 μL to about 135 μL. In some embodiments, the volume of the single dose in the reservoir is about 100 μL. In some embodiments, the volume of the single dose in the reservoir is about 133 μL. In some embodiments, the device is configured to deliver the single dose at a volume from about 90 μL to about 140 μL. In some embodiments, the device is configured to deliver the single dose at a volume from about 95 μL to about 135 μL. In some embodiments, the device is configured to deliver the single dose at a volume of about 100 μL. In some embodiments, the device is configured to deliver the single dose at a volume of about 133 μL.

In some embodiments, the device is actuatable with one hand. In some embodiments, the device is configured such that the 90% confidence interval for dose delivered per actuation is ±about 2%. In some embodiments, the device is configured such that the 95% confidence interval for dose delivered per actuation is ±about 2.5%. In some embodiments, the device is configured such that the delivery time is less than about 25 seconds. In some embodiments, the device is configured such that the delivery time is less than about 20 seconds.

In some embodiments, upon nasal delivery of said pharmaceutical composition to said subject, less than about 20% of said pharmaceutical composition leaves the nasal cavity via drainage into the nasopharynx or externally. In some embodiments, upon nasal delivery of said pharmaceutical composition to said subject, less than about 10% of said pharmaceutical composition leaves the nasal cavity via drainage into the nasopharynx or externally. In some embodiments, upon nasal delivery of said pharmaceutical composition to said subject, less than about 5% of said pharmaceutical composition leaves the nasal cavity via drainage into the nasopharynx or externally. In some embodiments, the plasma concentration versus time curve of the naloxone hydrochloride or hydrate thereof in said subject has a Tₘₐₓ of between about 10 and about 30 minutes.

In some embodiments, the subject exhibits one or more symptoms selected from the group consisting of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting. In some embodiments, the subject exhibits respiratory depression. In some embodiments, said respiratory depression is caused by the illicit use of opioids, or by an accidental misuse of opioids during medical opioid therapy. In some embodiments, said subject is free from respiratory depression for at least about 1 hour following treatment comprising essentially of delivery of said therapeutically effective amount of said opioid antagonist. In some embodiments, said subject is free from respiratory depression for at least about 2 hours following treatment comprising essentially of delivery of said therapeutically effective amount of said opioid antagonist. In some embodiments, said subject is free from respiratory depression for at least about 4 hours following treatment comprising essentially of delivery of said therapeutically effective amount of said opioid antagonist. In some embodiments, said subject is free from respiratory depression for at least about 6 hours following treatment comprising essentially of delivery of said therapeutically effective amount of said opioid antagonist. In some embodiments, said subject is in a lying, supine, or recovery position.

In some embodiments, said single actuation yields a plasma concentration of ≥0.2 ng/mL within 2.5 minutes in said subject. In some embodiments, said single actuation yields a plasma concentration of ≥1 ng/mL within 5 minutes in said subject. In some embodiments, said single actuation yields a plasma concentration of ≥3 ng/mL within 10 minutes in said subject. In some embodiments, said single actuation yields a plasma concentration of ≥0.2 ng/mL within 2.5 minutes in said subject. In some embodiments, said single actuation yields a plasma concentration of ≥1 ng/mL within 5 minutes in said subject. In some embodiments, the subject is a mammal. In some embodiments, the subject is a human. In some embodiments, said subject is an opioid overdose subject or a suspected opioid overdose subject.

In some embodiments, the pharmaceutical composition is administered as a spray of droplets to the subject. In some embodiments, the Dv90 of the spray of droplets is from about 48 μm to about 80 μm. In some embodiments, the Dv90 of the spray of droplets is from about 60 μm to about 80 μm. In some embodiments, the Dv90 of the droplets is from about 56.77 μm. In some embodiments, the Dv90 of the droplets is from about 55.10 μm. In some embodiments, the Dv50 of the droplets is from about 20 μm to about 40 μm. In some embodiments, the Dv50 of the droplets is from about 25 μm to about 40 μm. In some embodiments, the Dv50 of the droplets is from about 22 μm to about 34 μm. In some embodiments, the Dv50 of the droplets is from about 24.72 μm. In some embodiments, the Dv50 of the droplets is from about 24.75 μm. In some embodiments, the Dv10 of the droplets is from about 10 μm to about 20 μm. In some embodiments, the Dv10 of the droplets is from about 12 μm to about 20 μm. In some embodiments, the Dv10 of the droplets is from about 10 μm to about 17 μm. In some embodiments, the Dv10 of the droplets is about 11.35 μm. In some embodiments, the Dv10 of the droplets is about 11.47 μm. In some embodiments, the percent volume of droplets less than 10 μm is less than about 12%. In some embodiments, the percent volume of droplets less than 10 μm is less than about 10%. In some embodiments, the percent volume of droplets less than 10 μm is about 6.7%. In some embodiments, the percent volume of droplets less than 10 μm is about 6.3%. In some embodiments, the device sprays a spray pattern with a Dmax of about 50 mm. In some embodiments, the device sprays a spray pattern with a Dmax of about 40.2 mm. In some embodiments, the device sprays a spray pattern with a Dmax of about 40.7 mm. In some embodiments, the device sprays a spray pattern with an area of about 750 mm² to about 1500 mm². In some embodiments, the device sprays a spray pattern with an area of about 1100 mm². In some embodiments, the device sprays a spray pattern with an area of about 1110 mm². In some embodiments, the device sprays a spray pattern with an ovality ratio of about 1.0 to about 2.5. In some embodiments, the device sprays a spray pattern with an ovality ratio of about 1.2.

In some embodiments, said device is filled with said pharmaceutical composition using sterile filling.

In some embodiments, said pharmaceutical composition is storage-stable for about twelve months at about 25° C. and about 60% relative humidity.

In some embodiments, said device is a single-dose device, wherein said pharmaceutical composition is present in one reservoir, and wherein said therapeutically effective amount of said opioid antagonist is delivered essentially by one actuation of said device into one nostril of said patient.

In some embodiments, about 100 μL of said pharmaceutical composition is delivered by said actuation. In some embodiments, about 133 μL of said pharmaceutical composition is delivered by said actuation. In some embodiments, the volume of said pharmaceutical composition delivered by said actuation is selected from the group consisting of about 100 μL and about 133 μL.

In some embodiments, said device is actuatable with one hand.

In some embodiments, the delivery time is less than about 25 seconds. In some embodiments, the delivery time is less than about 20 seconds.

In some embodiments, the 90% confidence interval for dose delivered per actuation is ±about 2%. In some embodiments, the 95% confidence interval for dose delivered per actuation is ±about 2.5%.

In some embodiments, upon nasal delivery of said pharmaceutical composition to said patient, less than about 20% of said pharmaceutical composition leaves the nasal cavity via drainage into the nasopharynx or externally. In some embodiments, upon nasal delivery of said pharmaceutical composition to said patient, less than about 10% of said pharmaceutical composition leaves the nasal cavity via drainage into the nasopharynx or externally. In some embodiments, upon nasal delivery of said pharmaceutical composition to said patient, less than about 5% of said pharmaceutical composition leaves the nasal cavity via drainage into the nasopharynx or externally.

In some embodiments, the plasma concentration versus time curve of said opioid antagonist in said patient has a Tₘₐₓ of less than 30 minutes. In some embodiments, the plasma concentration versus time curve of said opioid antagonist in said patient has a Tₘₐₓ of less than 25 minutes. In some embodiments, the plasma concentration versus time curve of the opioid antagonist in the patient has a Tₘₐₓ of less than 20 minutes. In some embodiments, the plasma concentration versus time curve of said opioid antagonist in said patient has a Tₘₐₓ of about 20 minutes. In some embodiments, the plasma concentration versus time curve of the opioid antagonist in the patient has a Tₘₐₓ of less than 19 minutes. In some embodiments, the plasma concentration versus time curve of the opioid antagonist in the patient has a Tₘₐₓ of about 18.5 minutes.

In some embodiments, delivery of said therapeutically effective amount to said patient, provides occupancy at Tₘₐₓ of said opioid antagonist at the opioid receptors in the respiratory control center of said patient of greater than about 90%. In some embodiments, delivery of said therapeutically effective amount to said patient, provides occupancy at Tₘₐₓ of said opioid antagonist at the opioid receptors in the respiratory control center of said patient of greater than about 95%. In some embodiments, delivery of said therapeutically effective amount to said patient, provides occupancy at Tₘₐₓ of said opioid antagonist at the opioid receptors in the respiratory control center of said patient of greater than about 99%.

In some embodiments, said patient is free from respiratory depression for at least about 1 hour following treatment comprising essentially of delivery of said therapeutically effective amount of said opioid antagonist. In some embodiments, said patient is free from respiratory depression for at least about 2 hours following treatment comprising essentially of delivery of said therapeutically effective amount of said opioid antagonist. In some embodiments, said patient is free from respiratory depression for at least about 4 hours following treatment comprising essentially of delivery of said therapeutically effective amount of said opioid antagonist. In some embodiments, said patient is free from respiratory depression for at least about 6 hours following treatment comprising essentially of delivery of said therapeutically effective amount of said opioid antagonist.

In some embodiments, said patient is free from respiratory depression for at least about 1 hour following treatment comprising essentially of delivery of said therapeutically effective amount of said opioid antagonist. In some embodiments, said patient is free from respiratory depression for at least about 2 hours following treatment comprising essentially of delivery of said therapeutically effective amount of said opioid antagonist. In some embodiments, said patient is free from respiratory depression for at least about 4 hours following treatment comprising essentially of delivery of said therapeutically effective amount of said opioid antagonist. In some embodiments, said patient is free from respiratory depression for at least about 6 hours following treatment comprising essentially of delivery of said therapeutically effective amount of said opioid antagonist.

In some embodiments, the pharmaceutical composition contained in the device comprises about 4.4 mg naloxone hydrochloride dihydrate. In some embodiments, the pharmaceutical composition contained device comprises about 2.2 mg naloxone hydrochloride dihydrate. In some embodiments, the pharmaceutical composition contained in the device comprises about 4 mg naloxone hydrochloride dihydrate. In some embodiments, the pharmaceutical composition contained device comprises about 6 mg naloxone hydrochloride dihydrate. In some embodiments, the pharmaceutical composition contained device comprises about 8 mg naloxone hydrochloride dihydrate.

In some embodiments, upon nasal delivery of said pharmaceutical composition to said patient, less than about 10% of said pharmaceutical composition leaves the nasal cavity via drainage into the nasopharynx or externally.

In some embodiments, said device is actuatable with one hand.

In some embodiments, the delivery time is less than about 25 seconds. In some embodiments, the delivery time is less than about 20 seconds.

In some embodiments, the 90% confidence interval for dose delivered per actuation is ±about 2%. In some embodiments, the 95% confidence interval for dose delivered per actuation is ±about 2.5%.

In some embodiments, upon nasal delivery of said pharmaceutical composition to said patient, less than about 20% of said pharmaceutical composition leaves the nasal cavity via drainage into the nasopharynx or externally. In some embodiments, upon nasal delivery of said pharmaceutical composition to said patient, less than about 10% of said pharmaceutical composition leaves the nasal cavity via drainage into the nasopharynx or externally. In some embodiments, upon nasal delivery of said pharmaceutical composition to said patient, less than about 5% of said pharmaceutical composition leaves the nasal cavity via drainage into the nasopharynx or externally.

In some embodiments, said patient is free from respiratory depression for at least about 1 hour following treatment comprising essentially of delivery of said therapeutically effective amount of said opioid antagonist. In some embodiments, said patient is free from respiratory depression for at least about 2 hours following treatment comprising essentially of delivery of said therapeutically effective amount of said opioid antagonist. In some embodiments, said patient is free from respiratory depression for at least about 4 hours following treatment comprising essentially of delivery of said therapeutically effective amount of said opioid antagonist. In some embodiments, said patient is free from respiratory depression for at least about 6 hours following treatment comprising essentially of delivery of said therapeutically effective amount of said opioid antagonist.

In some embodiments, said device is filled with said pharmaceutical composition using sterile filling.

In some embodiments, said pharmaceutical composition is storage-stable for about twelve months at about 25° C. and about 60% relative humidity.

In some embodiments, said opioid antagonist is the only pharmaceutically active compound in said pharmaceutical composition.

Also provided are devices as recited in any of the preceding embodiments for use in the treatment of an opioid overdose symptom selected from: respiratory depression, postoperative opioid respiratory depression, altered level consciousness, miotic pupils, cardiovascular depression, hypoxemia, acute lung injury, aspiration pneumonia, sedation, and hypotension.

Also provided are devices as recited in any of the preceding embodiments for use in the reversal of respiratory depression induced by opioids.

In some embodiments, said respiratory depression is caused by the illicit use of opioids or by an accidental misuse of opioids during medical opioid therapy.

Also provided are devices as recited in any of the preceding embodiments for use in the complete or partial reversal of narcotic depression, including respiratory depression, induced by opioids selected from: natural and synthetic narcotics, propoxyphene, methadone, nalbuphine, pentazocine and butorphanol.

In some embodiments, said patient is an opioid overdose patient or a suspected opioid overdose patient.

In some embodiments, said patient is in a lying, supine, or recovery position. In some embodiments, said patient is in a lying position. In some embodiments, said patient is in a supine position. In some embodiments, said patient is in a recovery position.

In some embodiments, said therapeutically effective amount of an opioid antagonist is delivered by an untrained individual.

Also provided are kits comprising a device described herein and written instructions for using the device. Also provided are kits comprising a device described herein and an opioid agonist. In some embodiments the kit further comprises written instructions. In some embodiments, the opioid agonist is selected from codeine, morphine, methadone, fentanyl, oxycodone HCl, hydrocodone bitartrate, hydromorphone, oxymorphone, meperidine, propoxyphene, opium, heroin, and certain narcotic-antagonist analgesics, such as, nalbuphine, pentazocine and butorphanol. In some embodiments, the opioid agonist is selected from tapentadol and tramadol.

Also provided are embodiments wherein any embodiment above may be combined with any one or more of these embodiments, provided the combination is not mutually exclusive.

Tamper-proof and tamper-resistant formulating technologies have been developed for safer delivery of opioid antagonists, but such formulations are still abused resulting in opioid overdose. One such technology (Abuse Deterrent Prolonged Release Erosion Matrix (ADPREM); Egalet) utilizes a water-degradable polymer matrix technology that erodes from the surface at a constant rate. The matrix consists of one or more plasticizing polymers that cannot be crushed or melted. Another such technology (Abuse Resistant Technology (ART); Elite Laboratories) utilizes a proprietary coating technology consisting of various polymers that can sequester an opioid antagonist (naltrexone) in fragile micropellets that are indistinguishable from the pellets containing the opioid. The formulation is designed to release sequestered antagonist only if the dosage is crushed or otherwise damaged for extraction. Oral dosage forms are prepared by coating powders, crystals, granules, or pellets with various polymers to impart different characteristics. The formulations can release the active drug in both immediate and sustained release form. Chronodelivery formulations using this technology can effectively delay drug absorption for up to five hours. Aversion (Acura Pharmaceuticals) utilizes certain proprietary combinations of functional excipients (e.g., gelling agents) and active ingredients intended to discourage the most common methods of prescription drug misuse and abuse. Ingredients may include nasal irritants (e.g., capsaicin) and aversive agents (e.g., niacin). In some embodiments, the opioid agonist is in a tamper-proof formulation. In some embodiments, the opioid agonist is in a tamper-resistant formulation. In some embodiments, the opioid agonist is selected from ACUROX® Oxycodone DETERx®, Egalet hydrocodone, Egalet morphine, Egalet oxycodone, EXALGO®, OPANA®, and REMOXY®.

### Methods of Treatment

Also provided are devices for use in treating opioid overdose and symptoms thereof and methods of using the devices. Naloxone prevents or reverses the effects of opioids including respiratory depression, sedation and hypotension. Also, it can reverse the psychotomimetic and dysphoric effects of agonist-antagonists such as pentazocine. Naloxone causes abrupt reversal of narcotic depression which may result in nausea, vomiting, sweating, tachycardia, increased blood pressure, tremulousness, seizures and cardiac arrest, however, there is no clinical experience with naloxone hydrochloride overdosage in humans. In the mouse and rat the intravenous LD₅₀ is 150±5 mg/kg and 109±4 mg/kg respectively. In acute subcutaneous toxicity studies in newborn rats the LD50 (95% CL) is 260 (228-296) mg/kg. Subcutaneous injection of 100 mg/kg/day in rats for 3 weeks produced only transient salivation and partial ptosis following injection: no toxic effects were seen at 10 mg/kg/day for 3 weeks.

Naloxone hydrochloride injection is indicated for the complete or partial reversal of narcotic depression, including respiratory depression, induced by opioids selected from: natural and synthetic narcotics, propoxyphene, methadone, and certain narcotic-antagonist analgesics: nalbuphine, pentazocine and butorphanol. Naloxone hydrochloride is also indicated for the diagnosis of suspected acute opioid overdosage. For the treatment of known or suspected narcotic overdose in adults an initial dose of 0.4 mg to 2 mg of naloxone hydrochloride intravenously is indicated. If the desired degree of counteraction and improvement in respiratory functions is not obtained, administration may be repeated at 2 to 3 minute intervals. If no response is observed after 10 mg of naloxone hydrochloride have been administered, the diagnosis of narcotic-induced or partial narcotic-induced toxicity should be questioned. The usual initial dose in children is 0.01 mg/kg body weight given IV. If this dose does not result in the desired degree of clinical improvement, a subsequent dose of 0.1 mg/kg body weight may be administered. When using naloxone hydrochloride injection in neonates a product containing 0.02 mg/mL should be used.

It has also been reported that naloxone hydrochloride is an effective agent for the reversal of the cardiovascular and respiratory depression associated with narcotic and possibly some non-narcotic overdoses. The authors stated that due to naloxone's pharmacokinetic profile, a continuous infusion protocol is recommended when prolonged narcotic antagonist effects are required. (Handal et al., Ann Emerg Med. 1983 July; 12(7):438-45).

In one aspect, described herein is a method of treating an opioid overdose or symptom thereof in a subject in need thereof, comprising administering to the subject a pharmaceutical composition described herein.

In some embodiments, the pharmaceutical composition comprises a therapeutically effective amount of naloxone hydrochloride or a hydrate thereof. In some embodiments, the hydrate of naloxone hydrochloride is naloxone hydrochloride dihydrate. In some embodiments, the therapeutically effective amount is from about 2 mg to about 10 mg of the aqueous solution. In some embodiments, the therapeutically effective amount is from about 4 mg to about 8 mg. In some embodiments, the therapeutically effective amount is selected from the group consisting of about 4 mg, about 6 mg, and about 8 mg. In some embodiments, the therapeutically effective amount is about 4 mg. In some embodiments, the therapeutically effective amount is about 6 mg. In some embodiments, the therapeutically effective amount is about 8 mg.

In some embodiments, upon nasal delivery of said pharmaceutical composition to said subject, less than about 20% of said pharmaceutical composition leaves the nasal cavity via drainage into the nasopharynx or externally. In some embodiments, the upon nasal delivery of said pharmaceutical composition to said subject, less than about 10% of said pharmaceutical composition leaves the nasal cavity via drainage into the nasopharynx or externally. In some embodiments, the upon nasal delivery of said pharmaceutical composition to said subject, less than about 5% of said pharmaceutical composition leaves the nasal cavity via drainage into the nasopharynx or externally.

In some embodiments, the plasma concentration versus time curve of said naloxone hydrochloride or hydrate thereof in said subject has a Tₘₐₓ of between about 20 and about 30 minutes. In some embodiments, the administration yields a mean naloxone plasma concentration of ≥0.2 ng/mL within 2.5 minutes in said subject. In some embodiments, the administration yields a mean naloxone plasma concentration of ≥1 ng/mL within 5 minutes in said subject. In some embodiments, the administration yields a mean naloxone plasma concentration of ≥3 ng/mL within 10 minutes in said subject.

In some embodiments, the subject exhibits one or more symptoms selected from the group consisting of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting. In some embodiments, the subject exhibits respiratory depression. In some embodiments, the opioid overdose or symptom thereof is caused by an opioid selected from the group consisting of codeine, morphine, methadone, fentanyl, carfentanyl, acetyl fentanyl, oxycodone hydrochloride, hydrocodone bitartrate, hydromorphone, oxymorphone, meperidine, propoxyphene, opium, heroin, tramadol, tapentadol, and narcotic-antagonist analgesics. In some embodiments, the narcotic-antagonist analgesics is selected from the group consisting of nalbuphine, pentazocine, and butorphanol. In some embodiments, the subject is a mammal. In some embodiments, the subject is a human. In some embodiments, the subject is an opioid overdose subject or a suspected opioid overdose subject.

In some embodiments, the opioid overdose or symptom thereof is caused by a fentanyl derivative of the Formula (I)

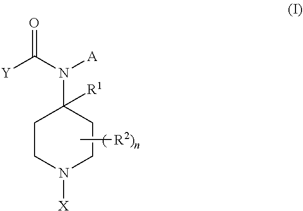

wherein A is aryl or heteroaryl optionally substituted with halo, C₁-C₃ alkyl, or C₁-C₃ alkoxy, X is C₁-C₃ alkyl or hydroxyethyl, optionally substituted with —COOCH₃, aryl, or heteroaryl optionally substituted with both C₁-C₃ alkyl and ═O, Y is C₁-C₄ alkyl, C₂-C₃ alkenyl, C₁-C₃ alkoxy, C₁-C₃ alkoxyalkyl, cycloalkyl, or heteroaryl, R1 and R2 are each independently selected from the group consisting of phenyl, C₁-C₃ alkyl, C₂-C₃ alkenyl, C₁-C₃ alkoxyalkyl, or C₁-C₃ alkoxy, and —COOCH₃, and n is 1, 2, or 3.

In some embodiments, the subject is a mammal. In some embodiments, the subject is a human. In some embodiments, the subject is an opioid overdose subject or a suspected opioid overdose subject.

In some embodiments, said opioid antagonist is the only pharmaceutically active compound in said pharmaceutical composition.

In some embodiments, said opioid antagonist is naloxone hydrochloride. In some embodiments, said opioid antagonist is naloxone hydrochloride dihydrate.

In some embodiments, said pharmaceutical composition comprises a solution of naloxone hydrochloride, or a hydrate thereof.

In some embodiments, said patient is an opioid overdose patient or a suspected opioid overdose patient.

In some embodiments, said patient is in a lying, supine, or recovery position. In some embodiments, said patient is in a lying position. In some embodiments, said patient is in a supine position. In some embodiments, said patient is in a recovery position.

In some embodiments, said therapeutically effective amount of an opioid antagonist is delivered by an untrained individual.

In some embodiments, said therapeutically effective amount is equivalent to about 4 mg to about 10 mg of naloxone hydrochloride. In some embodiments, said therapeutically effective amount is equivalent to an amount chosen from about 2 mg naloxone hydrochloride, about 4 mg of naloxone hydrochloride, and about 8 mg naloxone hydrochloride. In some embodiments, said therapeutically effective amount is equivalent to about 2 mg of naloxone hydrochloride. In some embodiments, said therapeutically effective amount is equivalent to about 4 mg of naloxone hydrochloride. In some embodiments, said therapeutically effective amount is equivalent to about 6 mg of naloxone hydrochloride. In some embodiments, said therapeutically effective amount is equivalent to about 8 mg of naloxone hydrochloride. In some embodiments, the therapeutically effective amount is equivalent to about 3.4 mg of naloxone hydrochloride.

In some embodiments, said therapeutically effective amount is about 2.2 mg to about 13.2 mg of naloxone hydrochloride dihydrate. In some embodiments, said therapeutically effective amount is about 4.4 mg to about 11 mg of naloxone hydrochloride dihydrate. In some embodiments, said therapeutically effective amount is an amount chosen from about 2.2 mg naloxone hydrochloride dihydrate, about 4.4 mg of naloxone hydrochloride dihydrate, and about 8.8 mg naloxone hydrochloride dihydrate. In some embodiments, said therapeutically effective amount is about 2.2 mg of naloxone hydrochloride dihydrate. In some embodiments, said therapeutically effective amount is about 4.4 mg of naloxone hydrochloride dihydrate. In some embodiments, said therapeutically effective amount is about 8.8 mg of naloxone hydrochloride dihydrate. In some embodiments, said therapeutically effective amount is about 4 mg of naloxone hydrochloride dihydrate. In some embodiments, said therapeutically effective amount is about 6 mg of naloxone hydrochloride dihydrate. In some embodiments, said therapeutically effective amount is about 8 mg of naloxone hydrochloride dihydrate.

In some embodiments, said symptom is chosen from respiratory depression and central nervous system depression.

In some embodiments, said patient exhibits any of unresponsiveness to stimulus, unconsciousness, stopped breathing; erratic or stopped pulse, choking or gurgling sounds, blue or purple fingernails or lips, slack or limp muscle tone, contracted pupils, and vomiting.

In some embodiments, said patient is not breathing.

In some embodiments, said patient is in a lying, supine, or recovery position.

In some embodiments, said patient is in a lying position.

In some embodiments, said patient is in a supine position.

In some embodiments, said patient is a recovery position.

In some embodiments, said therapeutically effective amount is equivalent to about 2 mg to about 10 mg of naloxone hydrochloride.

In some embodiments, said therapeutically effective amount is equivalent to an amount chosen from about 2 mg naloxone hydrochloride, about 4 mg of naloxone hydrochloride, and about 8 mg naloxone hydrochloride.

In some embodiments, said therapeutically effective amount is equivalent to about 2 mg of naloxone hydrochloride.

In some embodiments, said therapeutically effective amount is equivalent to about 4 mg of naloxone hydrochloride.

In some embodiments, said therapeutically effective amount is equivalent to about 8 mg of naloxone hydrochloride.

In some embodiments, said opioid antagonist is the only pharmaceutically active compound in said pharmaceutical composition.

In some embodiments, said opioid antagonist is naloxone hydrochloride.

In some embodiments, upon nasal delivery of said pharmaceutical composition to said patient, less than about 20% of said pharmaceutical composition leaves the nasal cavity via drainage into the nasopharynx or externally.

In some embodiments, upon nasal delivery of said pharmaceutical composition to said patient, less than about 10% of said pharmaceutical composition leaves the nasal cavity via drainage into the nasopharynx or externally.

In some embodiments, upon nasal delivery of said pharmaceutical composition to said patient, less than about 5% of said pharmaceutical composition leaves the nasal cavity via drainage into the nasopharynx or externally.

In some embodiments, the plasma concentration versus time curve of said opioid antagonist in said patient has a Tₘₐₓ of less than 30 minutes.

In some embodiments, the plasma concentration versus time curve of said opioid antagonist in said patient has a Tₘₐₓ of less than 25 minutes.

In some embodiments, the plasma concentration versus time curve of said opioid antagonist in said patient has a Tₘₐₓ of about 20 minutes.

In some embodiments, said opioid overdose symptom is selected from: respiratory depression, central nervous system depression, and cardiovascular depression.

In some embodiments, said opioid overdose symptom is respiratory depression induced by opioids.

In some embodiments, said respiratory depression is caused by the illicit use of opioids or by an accidental misuse of opioids during medical opioid therapy.

In some embodiments, said respiratory depression is induced by opioids selected from: natural and synthetic narcotics, propoxyphene, methadone, nalbuphine, pentazocine and butorphanol.

In some embodiments, said respiratory depression is induced by an opioid selected from codeine, morphine, methadone, fentanyl, oxycodone HCl, hydrocodone bitartrate, hydromorphone, oxymorphone, meperidine, propoxyphene, opium, heroin, tramadol, and tapentadol.

In some embodiments, said patient is free from respiratory depression for at least about 1 hour following treatment comprising essentially of delivery of said therapeutically effective amount of said opioid antagonist.

In some embodiments, said patient is free from respiratory depression for at least about 2 hours following treatment comprising essentially of delivery of said therapeutically effective amount of said opioid antagonist.

In some embodiments, said patient is free from respiratory depression for at least about 4 hours following treatment comprising essentially of delivery of said therapeutically effective amount of said opioid antagonist.

In some embodiments, said patient is free from respiratory depression for at least about 6 hours following treatment comprising essentially of delivery of said therapeutically effective amount of said opioid antagonist.

In one aspect, described herein is a method of treating an opioid overdose or a symptom thereof, comprising intranasally administering to the subject a pharmaceutical composition comprising greater than 4 mg of naloxone hydrochloride or a hydrate thereof and chlorobutanol.

In some embodiments, the pharmaceutical composition is an aqueous solution. In some embodiments, the pharmaceutical composition is administered to the subject in a single dose. In some embodiments, the concentration of naloxone hydrochloride is about 6 mg per 100 μL of aqueous solution. In some embodiments, the total mass of naloxone hydrochloride administered is not more than about 20 mg. In some embodiments, the total mass of the hydrate of naloxone hydrochloride administered is not more than about 20 mg. In some embodiments, the total administered volume is from about 30 μL to about 200 μL. In some embodiments, the total administered volume is from about 50 μL to about 150 μL.

In some embodiments, upon nasal delivery of said pharmaceutical composition to said subject, less than about 20% of said pharmaceutical composition leaves the nasal cavity via drainage into the nasopharynx or externally. In some embodiments, the upon nasal delivery of said pharmaceutical composition to said subject, less than about 10% of said pharmaceutical composition leaves the nasal cavity via drainage into the nasopharynx or externally. In some embodiments, the upon nasal delivery of said pharmaceutical composition to said subject, less than about 5% of said pharmaceutical composition leaves the nasal cavity via drainage into the nasopharynx or externally.

In some embodiments, the plasma concentration versus time curve of said naloxone hydrochloride or hydrate thereof in said subject has a Tₘₐₓ of between about 20 and about 30 minutes. In some embodiments, the administration yields a mean naloxone plasma concentration of ≥0.2 ng/mL within 2.5 minutes in said subject. In some embodiments, the administration yields a mean naloxone plasma concentration of ≥1 ng/mL within 5 minutes in said subject. In some embodiments, the administration yields a mean naloxone plasma concentration of ≥3 ng/mL within 10 minutes in said subject.

In some embodiments, the subject exhibits one or more symptoms selected from the group consisting of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting. In some embodiments, the subject exhibits respiratory depression. In some embodiments, the opioid overdose or symptom thereof is caused by an opioid selected from the group consisting of codeine, morphine, methadone, fentanyl, carfentanyl, acetyl fentanyl, oxycodone hydrochloride, hydrocodone bitartrate, hydromorphone, oxymorphone, meperidine, propoxyphene, opium, heroin, tramadol, tapentadol, and narcotic-antagonist analgesics. In some embodiments, the narcotic-antagonist analgesics is selected from the group consisting of nalbuphine, pentazocine, and butorphanol. In some embodiments, the subject is a mammal. In some embodiments, the subject is a human. In some embodiments, the subject is an opioid overdose subject or a suspected opioid overdose subject.

In some embodiments, the opioid overdose or symptom thereof is caused by a fentanyl derivative of the Formula (I)

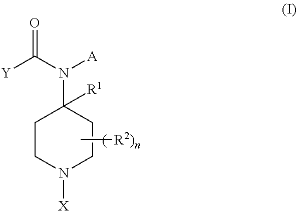

wherein A is aryl or heteroaryl optionally substituted with halo, C₁-C₃ alkyl, or C₁-C₃ alkoxy, X is C₁-C₃ alkyl or hydroxyethyl, optionally substituted with —COOCH₃, aryl, or heteroaryl optionally substituted with both C₁-C₃ alkyl and ═O, Y is C₁-C₄ alkyl, C₂-C₃ alkenyl, C₁-C₃ alkoxy, C₁-C₃ alkoxyalkyl, cycloalkyl, or heteroaryl, R1 and R2 are each independently selected from the group consisting of phenyl, C₁-C₃ alkyl, C₂-C₃ alkenyl, C₁-C₃ alkoxyalkyl, or C₁-C₃ alkoxy, and —COOCH₃, and n is 1, 2, or 3.

In some embodiments, the subject is a mammal. In some embodiments, the subject is a human. In some embodiments, the subject is an opioid overdose subject or a suspected opioid overdose subject.

In some embodiments, said opioid antagonist is the only pharmaceutically active compound in said pharmaceutical composition.

In some embodiments, said opioid antagonist is naloxone hydrochloride. In some embodiments, said opioid antagonist is naloxone hydrochloride dihydrate.

In some embodiments, said pharmaceutical composition comprises a solution of naloxone hydrochloride, or a hydrate thereof.

In some embodiments, said patient is an opioid overdose patient or a suspected opioid overdose patient.

In some embodiments, said patient is in a lying, supine, or recovery position. In some embodiments, said patient is in a lying position. In some embodiments, said patient is in a supine position. In some embodiments, said patient is in a recovery position.

In some embodiments, said therapeutically effective amount of an opioid antagonist is delivered by an untrained individual.

In some embodiments, said symptom is chosen from respiratory depression and central nervous system depression.

In some embodiments, said patient exhibits any of unresponsiveness to stimulus, unconsciousness, stopped breathing; erratic or stopped pulse, choking or gurgling sounds, blue or purple fingernails or lips, slack or limp muscle tone, contracted pupils, and vomiting.

In some embodiments, said patient is not breathing.

In some embodiments, said patient is in a lying, supine, or recovery position.

In some embodiments, said patient is in a lying position.

In some embodiments, said patient is in a supine position.

In some embodiments, said patient is a recovery position.

In some embodiments, said therapeutically effective amount is equivalent to about 2 mg to about 10 mg of naloxone hydrochloride.

In some embodiments, said therapeutically effective amount is equivalent to an amount chosen from about 2 mg naloxone hydrochloride, about 4 mg of naloxone hydrochloride, and about 8 mg naloxone hydrochloride.

In some embodiments, said therapeutically effective amount is equivalent to about 2 mg of naloxone hydrochloride.

In some embodiments, said therapeutically effective amount is equivalent to about 4 mg of naloxone hydrochloride.

In some embodiments, said therapeutically effective amount is equivalent to about 8 mg of naloxone hydrochloride.

In some embodiments, said opioid antagonist is the only pharmaceutically active compound in said pharmaceutical composition.

In some embodiments, said opioid antagonist is naloxone hydrochloride.

In some embodiments, upon nasal delivery of said pharmaceutical composition to said patient, less than about 20% of said pharmaceutical composition leaves the nasal cavity via drainage into the nasopharynx or externally.

In some embodiments, upon nasal delivery of said pharmaceutical composition to said patient, less than about 10% of said pharmaceutical composition leaves the nasal cavity via drainage into the nasopharynx or externally.

In some embodiments, upon nasal delivery of said pharmaceutical composition to said patient, less than about 5% of said pharmaceutical composition leaves the nasal cavity via drainage into the nasopharynx or externally.

In some embodiments, the plasma concentration versus time curve of said opioid antagonist in said patient has a Tₘₐₓ of less than 30 minutes.

In some embodiments, the plasma concentration versus time curve of said opioid antagonist in said patient has a Tₘₐₓ of less than 25 minutes.

In some embodiments, the plasma concentration versus time curve of said opioid antagonist in said patient has a Tₘₐₓ of about 20 minutes.

In some embodiments, said opioid overdose symptom is selected from: respiratory depression, central nervous system depression, and cardiovascular depression.

In some embodiments, said opioid overdose symptom is respiratory depression induced by opioids.

In some embodiments, said respiratory depression is caused by the illicit use of opioids or by an accidental misuse of opioids during medical opioid therapy.

In some embodiments, said respiratory depression is induced by opioids selected from: natural and synthetic narcotics, propoxyphene, methadone, nalbuphine, pentazocine and butorphanol.

In some embodiments, said respiratory depression is induced by an opioid selected from codeine, morphine, methadone, fentanyl, oxycodone HCl, hydrocodone bitartrate, hydromorphone, oxymorphone, meperidine, propoxyphene, opium, heroin, tramadol, and tapentadol.

In some embodiments, said patient is free from respiratory depression for at least about 1 hour following treatment comprising essentially of delivery of said therapeutically effective amount of said opioid antagonist.

In some embodiments, said patient is free from respiratory depression for at least about 2 hours following treatment comprising essentially of delivery of said therapeutically effective amount of said opioid antagonist.

In some embodiments, said patient is free from respiratory depression for at least about 4 hours following treatment comprising essentially of delivery of said therapeutically effective amount of said opioid antagonist.

In some embodiments, said patient is free from respiratory depression for at least about 6 hours following treatment comprising essentially of delivery of said therapeutically effective amount of said opioid antagonist.

In one aspect, described herein is a method of treating an opioid overdose or a symptom thereof, comprising intranasally administering to the subject a pharmaceutical composition comprising greater than 4 mg of naloxone hydrochloride or a hydrate thereof and glycerin.

In some embodiments, the pharmaceutical composition is an aqueous solution. In some embodiments, the pharmaceutical composition is administered to the subject in a single dose. In some embodiments, the concentration of naloxone hydrochloride is about 6 mg per 100 μL of aqueous solution. In some embodiments, the total mass of naloxone hydrochloride administered is not more than about 20 mg. In some embodiments, the total mass of the hydrate of naloxone hydrochloride administered is not more than about 20 mg. In some embodiments, the total administered volume is from about 30 μL to about 200 μL. In some embodiments, the total administered volume is from about 50 μL to about 150 μL.

In some embodiments, upon nasal delivery of said pharmaceutical composition to said subject, less than about 20% of said pharmaceutical composition leaves the nasal cavity via drainage into the nasopharynx or externally. In some embodiments, the upon nasal delivery of said pharmaceutical composition to said subject, less than about 10% of said pharmaceutical composition leaves the nasal cavity via drainage into the nasopharynx or externally. In some embodiments, the upon nasal delivery of said pharmaceutical composition to said subject, less than about 5% of said pharmaceutical composition leaves the nasal cavity via drainage into the nasopharynx or externally.

In some embodiments, the plasma concentration versus time curve of said naloxone hydrochloride or hydrate thereof in said subject has a Tₘₐₓ of between about 20 and about 30 minutes. In some embodiments, the administration yields a mean naloxone plasma concentration of ≥0.2 ng/mL within 2.5 minutes in said subject. In some embodiments, the administration yields a mean naloxone plasma concentration of ≥1 ng/mL within 5 minutes in said subject. In some embodiments, the administration yields a mean naloxone plasma concentration of ≥3 ng/mL within 10 minutes in said subject.

In some embodiments, the subject exhibits one or more symptoms selected from the group consisting of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting. In some embodiments, the subject exhibits respiratory depression. In some embodiments, the opioid overdose or symptom thereof is caused by an opioid selected from the group consisting of codeine, morphine, methadone, fentanyl, carfentanyl, acetyl fentanyl, oxycodone hydrochloride, hydrocodone bitartrate, hydromorphone, oxymorphone, meperidine, propoxyphene, opium, heroin, tramadol, tapentadol, and narcotic-antagonist analgesics. In some embodiments, the narcotic-antagonist analgesics is selected from the group consisting of nalbuphine, pentazocine, and butorphanol. In some embodiments, the subject is a mammal. In some embodiments, the subject is a human. In some embodiments, the subject is an opioid overdose subject or a suspected opioid overdose subject.

In some embodiments, the opioid overdose or symptom thereof is caused by a fentanyl derivative of the Formula (I)

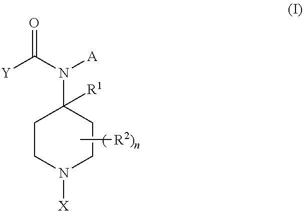

wherein A is aryl or heteroaryl optionally substituted with halo, C₁-C₃ alkyl, or C₁-C₃ alkoxy, X is C₁-C₃ alkyl or hydroxyethyl, optionally substituted with —COOCH₃, aryl, or heteroaryl optionally substituted with both C₁-C₃ alkyl and ═O, Y is C₁-C₄ alkyl, C₂-C₃ alkenyl, C₁-C₃ alkoxy, C₁-C₃ alkoxyalkyl, cycloalkyl, or heteroaryl, R1 and R2 are each independently selected from the group consisting of phenyl, C₁-C₃ alkyl, C₂-C₃ alkenyl, C₁-C₃ alkoxyalkyl, or C₁-C₃ alkoxy, and —COOCH₃, and n is 1, 2, or 3.

In some embodiments, the subject is a mammal. In some embodiments, the subject is a human. In some embodiments, the subject is an opioid overdose subject or a suspected opioid overdose subject.

In some embodiments, said opioid antagonist is the only pharmaceutically active compound in said pharmaceutical composition.

In some embodiments, said opioid antagonist is naloxone hydrochloride. In some embodiments, said opioid antagonist is naloxone hydrochloride dihydrate.

In some embodiments, said pharmaceutical composition comprises a solution of naloxone hydrochloride, or a hydrate thereof.

In some embodiments, said patient is an opioid overdose patient or a suspected opioid overdose patient.

In some embodiments, said patient is in a lying, supine, or recovery position. In some embodiments, said patient is in a lying position. In some embodiments, said patient is in a supine position. In some embodiments, said patient is in a recovery position.

In some embodiments, said therapeutically effective amount of an opioid antagonist is delivered by an untrained individual.

In some embodiments, said symptom is chosen from respiratory depression and central nervous system depression.

In some embodiments, said patient exhibits any of unresponsiveness to stimulus, unconsciousness, stopped breathing; erratic or stopped pulse, choking or gurgling sounds, blue or purple fingernails or lips, slack or limp muscle tone, contracted pupils, and vomiting.

In some embodiments, said patient is not breathing.

In some embodiments, said patient is in a lying, supine, or recovery position.

In some embodiments, said patient is in a lying position.

In some embodiments, said patient is in a supine position.

In some embodiments, said patient is a recovery position.

In some embodiments, said therapeutically effective amount is equivalent to about 2 mg to about 10 mg of naloxone hydrochloride.

In some embodiments, said therapeutically effective amount is equivalent to an amount chosen from about 2 mg naloxone hydrochloride, about 4 mg of naloxone hydrochloride, and about 8 mg naloxone hydrochloride.

In some embodiments, said therapeutically effective amount is equivalent to about 2 mg of naloxone hydrochloride.

In some embodiments, said therapeutically effective amount is equivalent to about 4 mg of naloxone hydrochloride.

In some embodiments, said therapeutically effective amount is equivalent to about 8 mg of naloxone hydrochloride.

In some embodiments, said opioid antagonist is the only pharmaceutically active compound in said pharmaceutical composition.

In some embodiments, said opioid antagonist is naloxone hydrochloride.

In some embodiments, upon nasal delivery of said pharmaceutical composition to said patient, less than about 20% of said pharmaceutical composition leaves the nasal cavity via drainage into the nasopharynx or externally.

In some embodiments, upon nasal delivery of said pharmaceutical composition to said patient, less than about 10% of said pharmaceutical composition leaves the nasal cavity via drainage into the nasopharynx or externally.

In some embodiments, upon nasal delivery of said pharmaceutical composition to said patient, less than about 5% of said pharmaceutical composition leaves the nasal cavity via drainage into the nasopharynx or externally.

In some embodiments, the plasma concentration versus time curve of said opioid antagonist in said patient has a Tₘₐₓ of less than 30 minutes.

In some embodiments, the plasma concentration versus time curve of said opioid antagonist in said patient has a Tₘₐₓ of less than 25 minutes.

In some embodiments, the plasma concentration versus time curve of said opioid antagonist in said patient has a Tₘₐₓ of about 20 minutes.

In some embodiments, said opioid overdose symptom is selected from: respiratory depression, central nervous system depression, and cardiovascular depression.

In some embodiments, said opioid overdose symptom is respiratory depression induced by opioids.

In some embodiments, said respiratory depression is caused by the illicit use of opioids or by an accidental misuse of opioids during medical opioid therapy.

In some embodiments, said respiratory depression is induced by opioids selected from: natural and synthetic narcotics, propoxyphene, methadone, nalbuphine, pentazocine and butorphanol.

In some embodiments, said respiratory depression is induced by an opioid selected from codeine, morphine, methadone, fentanyl, oxycodone HCl, hydrocodone bitartrate, hydromorphone, oxymorphone, meperidine, propoxyphene, opium, heroin, tramadol, and tapentadol.

In some embodiments, said patient is free from respiratory depression for at least about 1 hour following treatment comprising essentially of delivery of said therapeutically effective amount of said opioid antagonist.

In some embodiments, said patient is free from respiratory depression for at least about 2 hours following treatment comprising essentially of delivery of said therapeutically effective amount of said opioid antagonist.

In some embodiments, said patient is free from respiratory depression for at least about 4 hours following treatment comprising essentially of delivery of said therapeutically effective amount of said opioid antagonist.

In some embodiments, said patient is free from respiratory depression for at least about 6 hours following treatment comprising essentially of delivery of said therapeutically effective amount of said opioid antagonist.

Also provided are the devices, pharmaceutical compositions, kits, and methods of treatment described herein for use in the treatment of an opioid overdose symptom selected from: respiratory depression, postoperative opioid respiratory depression, altered level consciousness, miotic pupils, cardiovascular depression, hypoxemia, acute lung injury, aspiration pneumonia, sedation, and hypotension. Also provided are the devices, pharmaceutical compositions, kits, and methods of treatment described herein for use in the reversal of respiratory depression induced by opioids. In some embodiments, the respiratory depression is caused by the illicit use of opioids or by an accidental misuse of opioids during medical opioid therapy. Also provided are the devices, pharmaceutical compositions, kits, and methods of treatment described herein for use in the complete or partial reversal of narcotic depression, including respiratory depression, induced by opioids selected from: natural and synthetic narcotics, propoxyphene, methadone, nalbuphine, pentazocine and butorphanol. In some embodiments, narcotic depression, including respiratory depression, is induced by an opioid agonist selected from codeine, morphine, methadone, fentanyl, oxycodone HCl, hydrocodone bitartrate, hydromorphone, oxymorphone, meperidine, propoxyphene, opium, heroin, tramadol, and tapentadol.

Also provided are devices, pharmaceutical formulations, and kits for, and methods of, treating opioid overdose or a symptom thereof, comprising nasally administering to a patient in need thereof a pharmaceutical composition described herein, e.g., a pharmaceutical composition comprising a therapeutically effective amount of an opioid antagonist selected from naloxone and pharmaceutically acceptable salts and hydrates thereof, wherein the therapeutically effective amount is equivalent to about 2 mg to about 12 mg of naloxone hydrochloride. In some embodiments, the patient is not breathing. In some embodiments, the nasally administering is accomplished using a device described herein. In some embodiments, the opioid overdose symptom is selected from: respiratory depression, postoperative opioid respiratory depression, altered level consciousness, miotic pupils, cardiovascular depression, hypoxemia, acute lung injury, aspiration pneumonia, sedation, and hypotension. In some embodiments, the opioid overdose symptom is respiratory depression induced by opioids. In some embodiments, the respiratory depression is caused by the illicit use of opioids or by an accidental misuse of opioids during medical opioid therapy. In some embodiments, the respiratory depression is induced by opioids selected from: natural and synthetic narcotics, propoxyphene, methadone, nalbuphine, pentazocine and butorphanol. In some embodiments, the respiratory depression is induced by an opioid agonist selected from codeine, morphine, methadone, fentanyl, oxycodone HCl, hydrocodone bitartrate, hydromorphone, oxymorphone, meperidine, propoxyphene, opium, heroin, tramadol, and tapentadol.

Also provided are devices, kits, and pharmaceutical formulations for, and methods of, treating opioid overdose or a symptom thereof, comprising nasally administering to a patient in need thereof a pharmaceutical composition comprising a therapeutically effective amount of an opioid antagonist together and at least one known pharmaceutical agent. In some embodiments, the method comprises nasally administering to a patient in need thereof therapeutically effective amounts of a short-acting opioid antagonist and a long-acting opioid antagonist. In some embodiments, the method comprises nasally administering to a patient in need thereof therapeutically effective amounts of naloxone and naltrexone. In some embodiments, the method comprises nasally administering to a patient in need thereof therapeutically effective amounts of naloxone and methylnaltrexone. In some embodiments, the method comprises nasally administering to a patient in need thereof therapeutically effective amounts of naloxone and nalmefene.

Also provided are devices, kits, and pharmaceutical formulations for, and methods of, reversing the psychotomimetic and dysphoric effects of agonist-antagonists such as pentazocine, comprising nasally administering to a patient in need thereof a pharmaceutical composition described herein, e.g., a pharmaceutical composition comprising a therapeutically effective amount of an opioid antagonist selected from naloxone and pharmaceutically acceptable salts thereof, wherein the therapeutically effective amount is equivalent to about 2 mg to about 12 mg of naloxone hydrochloride or naloxone hydrochloride dihydrate. In some embodiments, the therapeutically effective amount is equivalent to about 4.4 mg of naloxone hydrochloride. In some embodiments, the therapeutically effective amount is equivalent to about 4 mg of naloxone hydrochloride dihydrate. In some embodiments, the therapeutically effective amount is equivalent to about 6 mg of naloxone hydrochloride dihydrate. In some embodiments, the therapeutically effective amount is equivalent to about 8 mg of naloxone hydrochloride dihydrate. In some embodiments, the nasally administering is accomplished using a device described herein.

Also provided are devices, kits, and pharmaceutical formulations for, and methods of, diagnosis of suspected acute opioid overdosage, comprising nasally administering to a patient in need thereof a pharmaceutical composition described herein, e.g., a pharmaceutical composition comprising a therapeutically effective amount of an opioid antagonist selected from naloxone and pharmaceutically acceptable salts thereof, wherein the therapeutically effective amount is equivalent to about 2 mg to about 12 mg of naloxone hydrochloride. In some embodiments, the therapeutically effective amount is equivalent to about 4 mg of naloxone hydrochloride. In some embodiments, the therapeutically effective amount is equivalent to about 4.4 mg of naloxone hydrochloride dihydrate. In some embodiments, the nasally administering is accomplished using a device described herein.

Also provided are devices, kits, and pharmaceutical formulations for, and methods of, treating opioid addiction, comprising nasally administering to a patient in need thereof a pharmaceutical composition described herein, e.g., a pharmaceutical composition comprising a therapeutically effective amount of an opioid antagonist selected from naloxone and pharmaceutically acceptable salts thereof, wherein the therapeutically effective amount is equivalent to about 2 mg to about 12 mg of naloxone hydrochloride. In some embodiments, the therapeutically effective amount is equivalent to about 4 mg of naloxone hydrochloride. In some embodiments, the therapeutically effective amount is equivalent to about 4.4 mg of naloxone hydrochloride dihydrate. In some embodiments, the therapeutically effective amount is equivalent to about 4 mg of naloxone hydrochloride dihydrate. In some embodiments, the therapeutically effective amount is equivalent to about 6 mg of naloxone hydrochloride dihydrate. In some embodiments, the therapeutically effective amount is equivalent to about 8 mg of naloxone hydrochloride dihydrate. In some embodiments, the nasally administering is accomplished using a device described herein.

Also provided are devices, kits, and pharmaceutical formulations for, and methods of, treating septic shock, comprising nasally administering to a patient in need thereof a pharmaceutical composition described herein, e.g., a pharmaceutical composition comprising a therapeutically effective amount of an opioid antagonist selected from naloxone and pharmaceutically acceptable salts thereof, wherein the therapeutically effective amount is equivalent to about 2 mg to about 12 mg of naloxone hydrochloride. In some embodiments, the therapeutically effective amount is equivalent to about 4 mg of naloxone hydrochloride. In some embodiments, the therapeutically effective amount is equivalent to about 4.4 mg of naloxone hydrochloride dihydrate. In some embodiments, the therapeutically effective amount is equivalent to about 4 mg of naloxone hydrochloride dihydrate. In some embodiments, the therapeutically effective amount is equivalent to about 6 mg of naloxone hydrochloride dihydrate. In some embodiments, the therapeutically effective amount is equivalent to about 8 mg of naloxone hydrochloride dihydrate. In some embodiments, the nasally administering is accomplished using a device described herein.

Also provided are devices, kits, and pharmaceutical formulations for, and methods of, treating opioid overdose or a symptom thereof, reversing the psychotomimetic and dysphoric effects of agonist-antagonists such as pentazocine, diagnosing suspected acute opioid overdosage, treating opioid addiction, or treating septic shock, comprising nasally administering to a patient in need thereof a pharmaceutical composition described herein, e.g., a pharmaceutical composition comprising a therapeutically effective amount of an opioid antagonist, wherein the therapeutically effective amount is about 2 mg to about 12 mg. In some embodiments, the therapeutically effective amount is equivalent to about 4.4 mg of naloxone hydrochloride dihydrate. In some embodiments, the therapeutically effective amount is equivalent to about 4 mg of naloxone hydrochloride dihydrate. In some embodiments, the therapeutically effective amount is equivalent to about 6 mg of naloxone hydrochloride dihydrate. In some embodiments, the therapeutically effective amount is equivalent to about 8 mg of naloxone hydrochloride dihydrate. In some embodiments, the therapeutically effective amount is equivalent to about 4 mg of naloxone hydrochloride. In some embodiments, the patient is an opioid overdose patient. In some embodiments, the patient is not breathing. In some embodiments, the opioid antagonist is the only pharmaceutically active compound in said pharmaceutical composition. In some embodiments, the opioid antagonist is selected from naltrexone, methylnaltrexone, and nalmefene, and pharmaceutically acceptable salts thereof. In some embodiments, the opioid antagonist is naltrexone hydrochloride. In some embodiments, the opioid antagonist is methylnaltrexone bromide. In some embodiments, the opioid antagonist is nalmefene hydrochloride. In some embodiments, the nasally administering is accomplished using a device described herein. In some embodiments, the opioid overdose symptom is selected from: respiratory depression, postoperative opioid respiratory depression, altered level consciousness, miotic pupils, cardiovascular depression, hypoxemia, acute lung injury, aspiration pneumonia, sedation, and hypotension. In some embodiments, the opioid overdose symptom is respiratory depression induced by opioids. In some embodiments, the respiratory depression is caused by the illicit use of opioids or by an accidental misuse of opioids during medical opioid therapy. In some embodiments, the respiratory depression is induced by opioids selected from: natural and synthetic narcotics, propoxyphene, methadone, nalbuphine, pentazocine and butorphanol. In some embodiments, the respiratory depression is induced by an opioid agonist selected from codeine, morphine, methadone, fentanyl, oxycodone HCl, hydrocodone bitartrate, hydromorphone, oxymorphone, meperidine, propoxyphene, opium, heroin, tramadol, and tapentadol.

Various eating disorders, including binge eating, bulimia, and stimulus-induced over-eating, develop because the behaviors are reinforced by the opioidergic system so often and so well that the person no longer can control the behavior. Thus eating disorders resemble opiate addiction and alcoholism. Accordingly, also provided are devices, kits, and pharmaceutical formulations for, and methods of, treating an eating disorder selected from binge eating, bulimia, and stimulus-induced over-eating, comprising nasally administering to a patient in need thereof a pharmaceutical composition described herein, e.g., a pharmaceutical composition comprising a therapeutically effective amount of an opioid antagonist, wherein the therapeutically effective amount is about 2 mg to about 12 mg. In some embodiments, the therapeutically effective amount is equivalent to about 4 mg of naloxone hydrochloride. In some embodiments, the therapeutically effective amount is equivalent to about 4.4 mg of naloxone hydrochloride. In some embodiments, the therapeutically effective amount is equivalent to about 4 mg of naloxone hydrochloride dihydrate. In some embodiments, the therapeutically effective amount is equivalent to about 6 mg of naloxone hydrochloride dihydrate. In some embodiments, the therapeutically effective amount is equivalent to about 8 mg of naloxone hydrochloride dihydrate. In some embodiments, the opioid antagonist is the only pharmaceutically active compound in said pharmaceutical composition. In some embodiments, the opioid antagonist is selected from naltrexone, methylnaltrexone, and nalmefene, and pharmaceutically acceptable salts thereof. In some embodiments, the opioid antagonist is naltrexone hydrochloride. In some embodiments, the opioid antagonist is methylnaltrexone bromide. In some embodiments, the opioid antagonist is nalmefene hydrochloride. In some embodiments, the nasally administering is accomplished using a device described herein.

It will be appreciated by those skilled in the art that changes could be made to the exemplary embodiments shown and described above without departing from the broad inventive concept thereof. It is understood, therefore, that the described epoxy resin compositions and methods are not limited to the exemplary embodiments shown and described, but it is intended to cover modifications within the spirit and scope of the disclosure and as defined by the claims. For example, specific features of the exemplary embodiments may or may not be part of the claimed invention and features of the disclosed embodiments may be combined. Unless specifically set forth herein, the terms “a”, “an” and “the” are not limited to one element but instead should be read as meaning “at least one”.

It is to be understood that at least some of the descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the method does not rely on the particular order of steps set forth herein, the particular order of the steps should not be construed as limitation on the claims. The claims directed to the methods described herein should not be limited to the performance of their steps in the order written, and one skilled in the art can readily appreciate that the steps may be varied and still remain within the spirit and scope of the present invention.

### EXAMPLES

In order that the invention described herein may be more fully understood, the following examples are set forth. The examples described in this application are offered to illustrate the aspects of the invention and their embodiments provided herein and are not to be construed in any way as limiting their scope. In the non-limiting examples below and accompanying figures, “chlorobutanol” and “chlorobutanol hemihydrate” are synonymous, i.e., they both refer to the hemihydrate of 1,1,1-trichloro-2-mehtylpropan-2-ol.

### Abbreviations

API: Active pharmaceutical ingredient
DOE: Design of experiment
NT: Not tested
Q.S.: Quantity sufficient
NX: Naloxone hydrochloride dihydrate
RH: Relative humidity
USP-PW: United States Pharmacopeia-purified water
RLD: reference listed drug
RC: Related compounds
NMT: Not more than

### Temp.: Temperature

### CB: Chlorobutanol

NA: Not available
AR: As required (to achieve the desired volume or weight)

### Example 1: Procedures for Characterization of Exemplary Formulations

### Nasal Spray Characterization Methods

pH

pH of the solution is measured by immersing a probe into the solution which is connected to a standard laboratory pH meter.

### Osmolality

Osmolality of the solution is determined by using an Advanced Instruments, Inc. osmometer equipped to measure osmolality by freezing point depression.

### Spray Pattern

The spray pattern of the spray is determined by the use of a Proveris Sprayview® system. To perform the testing, the nasal spray unit is positioned vertically. The tip of the nozzle of the nasal spray is positioned to be 30 mm from the laser beam of the SprayView system. The unit is automatically actuated using a Proveris Vereo automated actuator. The spray travels through the laser and the spray pattern the droplet size distribution is determined.

### Droplet Size Distribution

The droplet size distribution of the spray is determined by the use of a Malvern Spraytec® system. To perform the testing, the nasal spray unit is positioned vertically. The tip of the nozzle of the nasal spray is positioned to be 30 mm from the laser beam of the Spraytec® system. The unit is automatically actuated using a Proveris Vereo NSx automated actuator. The spray travels through the laser and the droplet size distribution is determined.

### Example 2: Exemplary Formulations and Properties

The below exemplary formulations were prepared and characterized based on the procedures described in Example 1.

TABLE 1a
Exemplary formulation A-1

| | Quantity | Quantity | |
|---|---|---|---|
| Ingredient | (mg/mL) | (mg/100 μL) | % w/w |
| Naloxone HCl Dihydrate | 40 | 4 | 4.0 |
| Chlorobutanol | 4.5 | 0.45 | 0.45 |
| Citric Acid, Anhydrous | 0.0857 | 0.009 | 0.009 |
| Trisodium Citrate Dihydrate | 0.124 | 0.01 | 0.01 |
| Sodium Chloride | 8.5 | 0.85 | 0.85 |
| Glycerin | 14 | 1.4 | 1.4 |
| Purified Water | AR | AR | AR |

Nominal pump delivery: 100 μL

TABLE 1b
Exemplary properties of formulation A-1

| Physical Test | Results |
|---|---|
| pH | 4.1 |
| Osmolality w/Glycerin | 613 |
| Osmolality w/o Glycerin | N/A |

TABLE 2
Exemplary spray pattern properties of formulation A-1

| | | | Actual Average | |
|---|---|---|---|---|
| | Range | | of 10 sprays | |
| Droplet Size Distribution | | | | |
| measured at 30 mm (from tip of | | | | |
| spray tip to laser) | | | | |
| Dv10 (μm) | 10-17 | | 11.35 | |
| Dv50 (μm) | 22-34 | | 24.72 | |
| Dv90 (μm) | 48-80 | | 56.77 | |
| % V < 10 μm (%) | 0-12% | | 6.7% | |
| Spray Pattern measured at | | | | |
| 30 mm (from tip of spray tip to | | | | |
| laser) | | | | |
| Dmax | MMT 50 | mm | 40.2 | mm |
| Area | 750-1500 | mm² | 1100 | mm² |

| | | |
|---|---|---|
| Ovality (ratio of longest to | 1.0-2.5 | 1.2 |
| shortest axis) | | |

TABLE 3a
Exemplary formulation B-1

| | Quantity | Quantity | |
|---|---|---|---|
| Ingredient | (mg/mL) | (mg/100 μL) | % w/w |
| Naloxone HCl Dihydrate | 60 | 6 | 6.0 |
| Chlorobutanol | 4.5 | 0.45 | 0.45 |
| Citric Acid, Anhydrous | 0.0857 | 0.009 | 0.009 |
| Trisodium Citrate Dihydrate | 0.124 | 0.01 | 0.01 |
| Sodium Chloride | 6.25 | 0.625 | 0.625 |
| Glycerin | 14 | 1.4 | 1.4 |
| Purified Water | AR | AR | AR |

Nominal pump delivery: 100 μL

TABLE 3b
Exemplary properties of formulation B-1

| Physical Test | Results |
|---|---|
| pH | 4.1 |
| Osmolality w/Glycerin | 607 |
| Osmolality w/o Glycerin | N/A |

TABLE 4
Exemplary spray pattern properties of formulation B-1

| | | | Actual Average | |
|---|---|---|---|---|
| | Range | | of 10 sprays | |
| Droplet Size Distribution | | | | |
| measured at 30 mm | | | | |
| Dv10 (μm) | 10-17 | | 11.47 | |
| Dv50 (μm) | 22-34 | | 24.75 | |
| Dv90 (μm) | 48-80 | | 55.10 | |
| % V < 10 μm (%) | 0-12% | | 6.3% | |
| Spray Pattern measured at | | | | |
| 30 mm (from tip of spray tip to | | | | |
| laser) | | | | |
| Dmax | MMT 50 | mm | 40.7 | mm |
| Area | 750-1500 | mm² | 1110 | mm² |

| | | |
|---|---|---|
| Ovality (ratio of longest to | 1.00-2.5 | 1.2 |
| shortest axis) | | |

TABLE 5a
Exemplary formulation C-1

| | Quantity | Quantity | |
|---|---|---|---|
| Ingredient | (mg/mL) | (mg/100 μL) | % w/w |
| Naloxone HCl Dihydrate | 60 | 6 | 6.0 |
| Chlorobutanol | 4.5 | 0.45 | 0.45 |
| Citric Acid, Anhydrous | 0.0857 | 0.009 | 0.009 |
| Trisodium Citrate Dihydrate | 0.124 | 0.01 | 0.01 |
| Sodium Chloride | 6.25 | 0.625 | 0.625 |
| Glycerin | 14 | 1.4 | 1.4 |
| Purified Water | AR | AR | AR |

Nominal pump delivery: 133 μL

TABLE 5b
Exemplary properties of formulation C-1

| Physical Test | Results |
|---|---|
| pH | 4.1 |
| Osmolality w/Glycerin | 607 |
| Osmolality w/o Glycerin | N/A |

### Example 3: Exemplary Development Work and Stability Studies

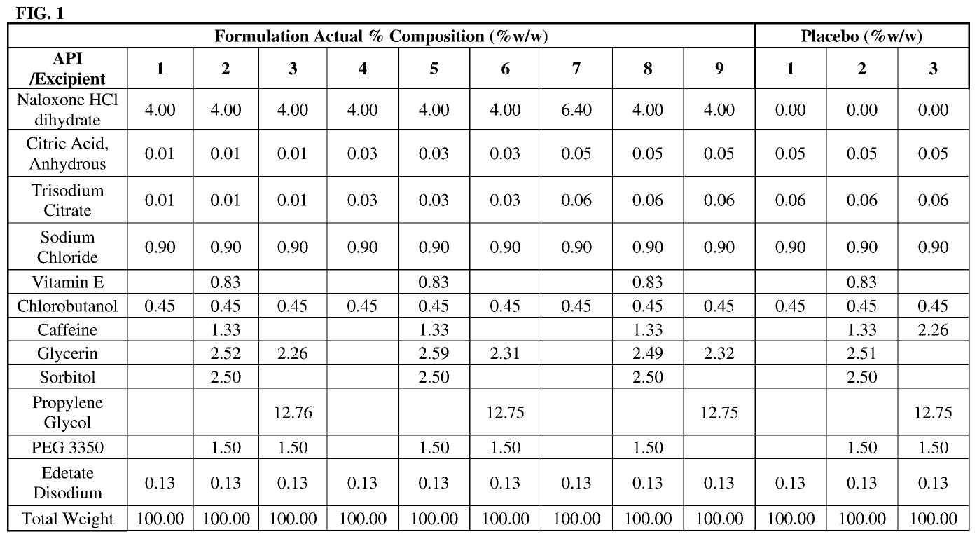

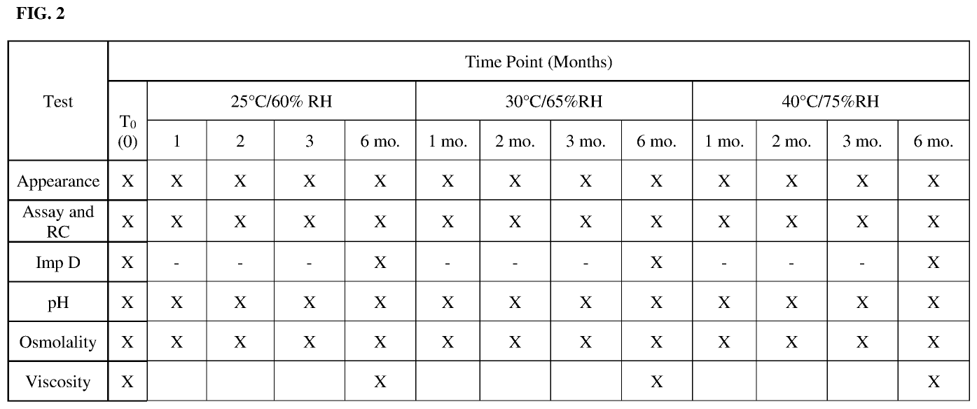

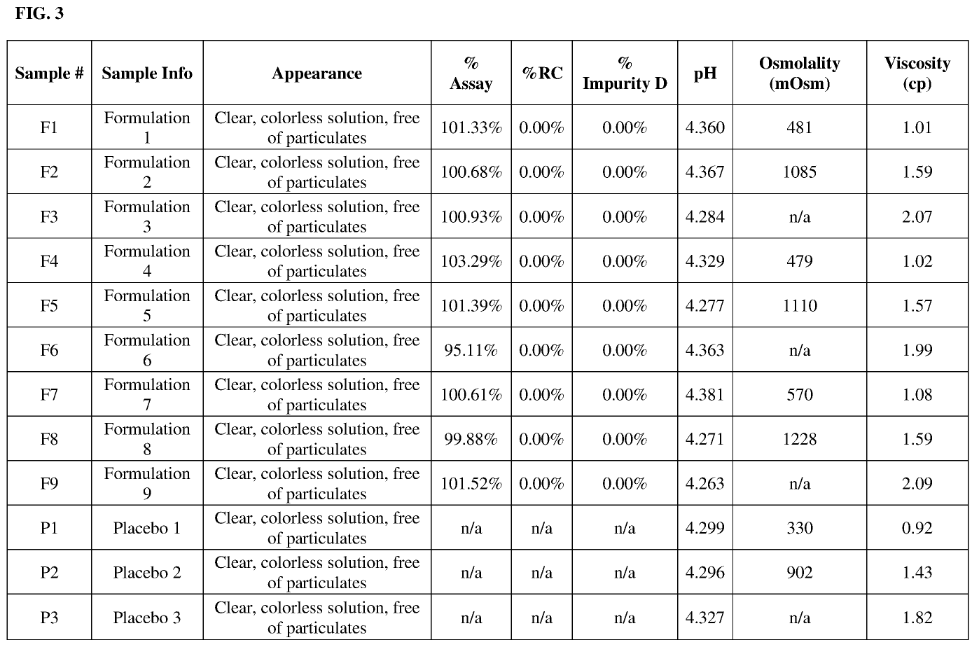

Nine Naloxone HCl dihydrate formulations and three placebo formulations were prepared and placed at stability conditions (25° C./60% RH, 30° C./65% RH, and 40° C./75% RH). FIG. 1 shows the composition of the formulations. Formulations and placebos were prepared in glove box filled with Nitrogen and stored in unit dose vials and HPLC vials blanketed with N2. Samples stored in UD vials will be used for all testing except viscosity test. Appearance testing, Assay and RC, Impurity D testing will be performed in duplicate. pH and osmolality will be measured once per formulation per condition. Samples stored in HPLC vials will be used for viscosity testing. Viscosity tests will be performed in triplicate. FIG. 2 shows the planned stability tests and dates for data measurements. FIG. 3 shows exemplary analytical data obtained from these formulations at an initial time T₀. FIG. 4 shows a summary of exemplary analytical data obtained from across different time points.

### Example 4: Studies of Exemplary Formulations F1(a), F1(b), F1(c), and F1(d)

Three Naloxone HCl dihydrate formulations, F1(a), F1(b), and F1(c), were prepared based on the pre-screening work performed. Table 6 shows the composition of the formulation. Formulations were tested for pH, osmolality and density.

Table 7 summarizes data for the three formulations.

Regression analyses were performed to observe the impact of the variation of EDTA and NaCl on Osmolality. No correlation was observed for the variation in EDTA; however, a strong correlation was observed for NaCl. Additionally, Two lots of Narcan Nasal Spray, 4 mg RLD, were tested for osmolality and pH.

Table 8 summarizes data for the two RLD samples.

Based on the observed correlation, a new formulation, F1(d), was prepared targeting an osmolality of 430 mOsm with 730 mg NaCl and a pH 4.3 using citric acid and trisodium citrate instead of citric acid and sodium hydroxide. The formulation is shown in Table 9. The test result of F1(d) is shown in Table 10a. A regression results for mg NaCl per 100 mL versus osmolality from all four formulation is shown in FIG. 5.

Chlorobutanol dissolves into water very slowly at ambient temperatures. Therefore during the preparation of F1(d), USP-PW was heated. After 18 minutes, the solution was clear and all of the chlorobutanol was completely dissolved.

TABLE 6
Formulation to Assess Effect of EDTA and
Sodium Chloride on Osmolality
Formulation Composition Wt (mg)

| API/Excipient | F1(a) | F1 (b) | F1 (c) |
|---|---|---|---|
| Naloxone | 4396 | 4396 | 4396 |
| Citric Acid, Anhydrous | 17.8 | 17.8 | 17.8 |
| Sodium Hydroxide | 4.9 | 4.9 | 4.9 |
| Sodium Chloride | 300 | 600 | 0 |
| Chlorobutanol | 450 | 450 | 450 |
| Edetate Disodium (EDTA) | 0 | 65 | 130 |

Adjusted pH to 4.3 with NaOH, Q.S. to 100 mL

TABLE 7
Test Results for Table Formulations

| | | pH | pH | | |
|---|---|---|---|---|---|
| | Sample | before | post | Osmolality | Density |
| Sample Info | ID | Q.S. | Q.S. | (mOsm) | (g/mL) |
| Formulation 1(a) | F1 (a) | 4.284 | 4.367 | 290, 294 | 1.02 |
| | | | | Average(292) | |
| Formulation 1(b) | F1 (b) | 4.302 | 4.440 | 387, 384 | 1.02 |
| | | | | Average(386) | |
| Formulation 1(c) | F1 (c) | 4.322 | 4.444 | 195, 197 | 1.02 |
| | | | | Average(196) | |

TABLE 8
Narcan NS, 4 mg Test Results

| Sample Info | pH | Osmolality (mOsm) |
|---|---|---|
| Narcan Nasal Spray, 4 mg, Lot 161184 | 4.492 | 427 |
| Narcan Nasal Spray, 4 mg, Lot 161184 | 4.608 | 419 |

TABLE 9
Formulation F1(d) - Targeting 430 mOsm and pH 4.3

| | F1 (d) Ingredients |
|---|---|
| API/Exipient | (mg) |
| Naloxone | 4396 |
| Citric Acid, Anhydrous | 6.8 |
| Trisodium Citrate Dihydrate | 16.8 |
| Sodium Chloride | 730 |
| Chlorobutanol | 450 |
| Edetate Disodium (EDTA) | 130 |

Adjusted pH to 4.3, q.s. to 100 mL

TABLE 10a
Formulation F1(d) Test Results

| | | pH before | pH post | Osmolality |
|---|---|---|---|---|
| Sample Info | Sample ID | Q.S. | Q.S. | (mOsm) |
| Formulation 1(d) | F1 (d) | 4.397 | 4.529 | 430, 434 |
| | | | | Average(432) |

### Example 5: Studies of Exemplary Formulation 1(e)

Formulation 1(e) was prepared to replicate Formulation 1(d). In Formulation 1(e), 70 ml of USP-PW was added initially instead of 60 ml. Formulation 1(e) was tested for pH. Table 10b summarizes data for this formulation.

TABLE 10b
pH Test Results for Formulation 1(e)

| Sample Info | Sample ID | pH before Q.S. | pH post Q.S. |
|---|---|---|---|
| Formulation 1(e) | F1 (e) | 4.427 | 4.451 |

### Example 6: Studies of Exemplary Formulation 1(f)

Formulation 1(f) was prepared. The Citric Acid and Trisodium Citrate was increased and decreased respectively to lower pH to 4.3 instead of 4.5. The trisodium citrate was added proportionally to determine the correct ratio. In the proposed formulation after reviewing the pH results in Formulation 1(e), the Citric Acid component was increased to 7.2 mg and the Trisodium Citrate Dihydrate component was decreased to 16.3 mg.

TABLE 11
Exemplary initial formulation 1(f) ingredients

| | F1(f) |
|---|---|
| | Ingredients |
| API/Excipient | (mg) |
| Naloxone | 4396 |
| Citric Acid, Anhydrous | 7.2 |
| Trisodium Citrate Dihydrate (initial addition) | 10.663 |
| Sodium Chloride | 730 |
| Chlorobutanol | 450 |
| Edetate Disodium (EDTA) | 130 |

Adjusted pH to 4.3, q.s. to 100 mL

Chlorobutanol, Sodium Chloride, EDTA, Naloxone HCl dihydrate, and Citric Acid were all added to 70 mL of USP-PW separately after each component was dissolved completely. The pH of this solution was measured to be 3.756 Table shows the amount of Trisodium Citrate Dihydrate additions and pH observation.

TABLE 12
Tri-sodium Additions for Formulation 1(f)

| Trisodium Citrate Amount (mg) | pH |
|---|---|
| 5.473 | 4.034 |
| 5.190 | 4.296 |
| 10.663 | =Total amount added |

Solution was q.s. to 100 ml with purified water. Formulation 1 (f) was tested for pH, and osmolality. Table 13 summarizes data for this formulation.

TABLE 13
Test Results for Formulation 1(f)

| | | pH before | pH post | Osmolality |
|---|---|---|---|---|
| Sample Info | Sample ID | Q.S. | Q.S. | (mOsm) |
| Formulation 1(f) | F1 (f) | 4.296 | 4.392 | 429, 427 |
| | | | | Average(428) |

### Example 7: Studies of Exemplary Formulation 1(g)

A variation to reduce Chlorobutanol from 4.5% to 2.5% was prepared. The proposed Formulation 1(g) is listed in Table 14.

TABLE 14
Exemplary initial formulation 1(g) ingredients

| API/Excipient | F1(g) Ingredients (mg) |
|---|---|
| Naloxone HCl Dihydrate | 4396 |
| Citric Acid, Anhydrous | 8 |
| Trisodium Citrate Dihydrate | 12.4 |
| Sodium Chloride | 730 |
| Chlorobutanol | 250 |
| Edetate Disodium (EDTA) | 130 |

Adjusted pH to 4.3, q.s. to 100 mL

### Example 8: Studies of Exemplary Formulations 1(h), 1(i), and 1(j)

Three additional formulations were made following the composition of Table 14. Two of the formulations were used for stability testing and micro testing and two of the formulations were used for extreme pH stability and nitrogen study. All formulations were pH adjusted to 4.3, q.s. to 100 mL with purified water, then tested for pH, osmolality and density. Table 15 summarizes physical test data for these four formulations.

TABLE 15
Formulation Test Results - 1(g), 1(h), 1(i), and 1(j)

| | | pH | pH | | |
|---|---|---|---|---|---|
| | | before | post | Osmolality | Density |
| Sample Info | Sample ID | Q.S. | Q.S. | (mOsm) | (g/mL) |
| Formulation 1(g) | F1 (g) | 4.150 | 4.347 | 427, 428 | 1.02 |
| | | | | Average(428) | |
| Formulation 1(h) | F1 (h) | 4.170 | 4.315 | 413, 416 | 1.02 |
| | | | | Average(415) | |
| Formulation 1(i) | F1 (i) | 4.143 | 4.298 | 420, 421 | 1.02 |
| | | | | Average(421) | |
| Formulation 1(j) | F1 (j) | 4.205 | 4.325 | 423, 426 | 1.02 |
| | | | | Average(425) | |

Formulation 1(h and i) was used for extreme pH 3.5 and 5.5 stability. 0.1N NaOH and 0.1N HCl will be used to adjust pH for the extreme pH ranges. Formulation 1(g) will be used for nitrogen overlay study.

### Extreme pH Adjustment

The pH was adjusted for Formulation 1(h) to approximately 3.5 and Formulation 1(i) to approximately 5.5 using 0.1N HCl and 0.1N NaOH, respectively. Refer to Table 12 for a summary of pH results.

During the addition of sodium hydroxide to Formulation 1 (i), white particles were observed to precipitate out into the solution. The particles quickly dissolved with stirring with a magnetic stir bar.

TABLE 16
pH of Extreme pH

| | | | | Amount of |
|---|---|---|---|---|
| | Sample | | Temperature | acid/base used |
| Sample Info | ID | pH | (° C.) | (ml) |
| Formulation 1(h) | F1 (h) | 3.504 | 21.2 | 1050 ml 0.1N HCl |
| Formulation 1(i) | F1 (i) | 5.492 | 21.3 | 1850 ml 0.1N NaOH |

### Sample Unit Dose Filling

96 unit dose vials were filled under ambient atmospheric conditions for each Formulations 1(g), 1(h), and 1(j) and appropriately labeled.

### Sample Unit Dose Nitrogen Filling

Inside a glovebox with reduced oxygen environment, filled 96 unit dose vials for each Formulation 1 (g, h, and i), and appropriately labeled.

### Chlorobutanol Full Scale Solubility Study

In Table 10a, the Chlorobutanol composition is 250 mg in 100 mL final volume (0.25%).

### Forced Degradation Prep

Weighed approximately 100 mg each of three different API vendor Naloxone HCl dihydrate into 20 mL scintillation vials.

### Example 9: Studies of Exemplary Formulations 1(k), 1(l), and 1(m)

### API Vendor Comparison

Formulations without Edetate Disodium (EDTA) were prepared. Table 20 shows the composition and the pH was varied to 3.7, 4.3, and 5.0 for stability assessment. Batches stored under nitrogen at 2-8° C.

TABLE 20
Formulations without EDTA

| API/Excipient | F1(p) | F1(q) | F1(r) | Variance (mg) |
|---|---|---|---|---|
| Target pH | 4.3 | 3.7 | 5.0 | NA |
| Actual pH | 4.366 | 3.830 | 4.970 | NA |
| Chlorobutanol | 250 | 250 | 250 | 1 |
| (mg) | | | | |
| Sodium Chloride | 730 | 730 | 730 | 1 |
| (mg) | | | | |
| Trisodium Citrate | 12.4 | 12.4 | 12.4 | 1 |
| Dihydrate (mg) | | | | |
| Citric Acid, | 8.13 | 9.01 | 7.11 | 0.15 |
| Anhydrous (mg) | | | | |
| Naloxone HCl | 4396 | 4396 | 4396 | 2 |
| dihydrate (mg) | | | | |

A nine batch DOE that assessed the impact of critical quality attributes bases on the impact of varying Glycerin amount, the pH, and assay. Additionally, the DOE assessed the impact of varying 02 headspace levels. Table 21 through Table 23 are the sample compositions with the addition of glycerin.

TABLE 21
DOE 1 - Batch 1 through 3

| | | | | Variance |
|---|---|---|---|---|
| API/Excipient | Batch 1 | Batch 2 | Batch 3 | (mg) |
| Target pH | 3.7 | 4.3 | 5.0 | NA |
| Chlorobutanol (mg) | 450 | 450 | 450 | 1 |
| Sodium Chloride (mg) | 730 | 730 | 730 | 1 |
| Trisodium Citrate | 12.4 | 12.4 | 12.4 | 1 |
| Dihydrate (mg) | | | | |
| Citric Acid, Anhydrous | 7.11 | 7.11 | 7.11 | 0.15 |
| (mg) | | | | |
| Naloxone HCl dihydrate | 4396 | 5495 | 6594 | 2 |
| (mg) | | | | |
| Adjusted pH | 3.707 | 4.288 | 4.978 | NA |
| Glycerin (mg) | 0.0 | 1100 | 2300 | NA |
| Observed pH | 3.720 | 4.321 | 5.001 | NA |
| Final pH | 3.893 | 4.374 | 5.001 | NA |
| Osmolality (mOsm) | 422 | 598 | 791 | NA |
| Density δ (mg/mL) | 1017.3177 | 1022.4168 | 1027.3191 | NA |
| Assay % | 100.1% | 100.5% | 100.5% | NA |
| O₂ Headspace Content % | <10 | <10 | <10 | NA |

TABLE 22
DOE 1 - Batch 4 through 6

| | | | | Variance |
|---|---|---|---|---|
| API/Excipient | Batch 4 | Batch 5 | Batch 6 | (mg) |
| Target pH | 3.7 | 4.3 | 5.0 | NA |
| Chlorobutanol (mg) | 450 | 450 | 450 | 1 |
| Sodium Chloride (mg) | 730 | 730 | 730 | 1 |
| Trisodium Citrate | 12.4 | 12.4 | 12.4 | 1 |
| Dihydrate (mg) | | | | |
| Citric Acid, Anhydrous | 7.11 | 7.11 | 7.11 | 0.15 |
| (mg) | | | | |
| Naloxone HCl dihydrate | 5495 | 6594 | 4396 | 2 |
| (mg) | | | | |
| Adjusted pH | 3.725 | 4.214 | 4.993 | NA |
| Glycerin (mg) | 2300 | 0.0 | 1100 | NA |
| Observed pH | 3.759 | 4.229 | 5.014 | NA |
| Final pH | 3.759 | 4.306 | 5.078 | NA |
| Osmolality (mOsm) | 756 | 502 | 559 | NA |
| Density δ (mg/mL) | 1024.1219 | 1023.1025 | 1020.5765 | NA |
| Assay % | 100.2% | 100.3% | 100.6% | NA |
| O₂ Headspace Content % | 13 | 13 | 13 | NA |

TABLE 23
DOE 1 - Batch 7 through 9

| | | | | Variance |
|---|---|---|---|---|
| API/Excipient | Batch 7 | Batch 8 | Batch 9 | (mg) |
| Target pH | 3.7 | 4.3 | 5.0 | NA |
| Chlorobutanol (mg) | 450 | 450 | 450 | 1 |
| Sodium Chloride (mg) | 730 | 730 | 730 | 1 |
| Trisodium Citrate | 12.4 | 12.4 | 12.4 | 1 |
| Dihydrate (mg) | | | | |
| Citric Acid, Anhydrous | 7.11 | 7.11 | 7.11 | 0.15 |
| (mg) | | | | |
| Naloxone HCl dihydrate | 6594 | 4396 | 5495 | 2 |
| (mg) | | | | |
| Adjusted pH | 3.722 | 4.248 | 4.977 | NA |
| Glycerin (mg) | 1100 | 2300 | 0.0 | NA |
| Observed pH | 3.742 | 4.272 | 4.981 | NA |
| Final pH | 3.786 | 4.346 | 5.000 | NA |
| Osmolality (mOsm) | 635 | 709 | 469 | NA |
| Density δ (mg/mL) | 1025.2463 | 1023.0185 | 1020.0047 | NA |
| Assay % | 100.8% | 100.4% | 100.3% | NA |
| O₂ Headspace Content % | 18 | 18 | 18 | NA |

From the DOE analysis of the initial test data, no impact of the variants were observed for assay (%) and related compounds (%). Osmolality varied as a function of both naloxone and glycerin concentration.

### Example 10: Exemplary Formulation Comprising Naloxone Hydrochloride Dihydrate

This example describes an aqueous formulation of naloxone hydrochloride dihydrate. The makeup and characteristics of this formulation are described in Tables 25 and 26.

TABLE 25
Makeup of exemplary formulation.

| | | Quan- | Composition | | |
|---|---|---|---|---|---|
| | | tity | | mg/ | Quantity |
| Ingredient | Function | (mg/mL) | % w/w | dose¹ | mg/100 mL |
| Naloxone | API | 131.88 | 12.703 | 13.188 | 13,188 |
| HCl | | | | | |
| Dihydrate | | | | | |
| Chloro- | Preserva- | 5.25 | 0.506 | 0.525 | 525 |
| butanol | tive | | | | |
| hemihydrate | | | | | |
| Citric Acid, | Buffer | 0.0857 | 0.008 | 0.00857 | 8.57 |
| Anhydrous | | | | | |
| Trisodium | Buffer | 0.154 | 0.015 | 0.0154 | 15.4 |
| Citrate | | | | | |
| Dihydrate | | | | | |
| Glycerin | Viscosity | 14 | 1.349 | 1.4 | 1,400 |
| | Adjust- | | | | |
| | ment | | | | |
| Purified | Carrier | 886.777 | 85.419 | 88.678 | 88,678 |
| Water | | | | | |

| | | | | |
|---|---|---|---|---|
| Total | 1038.1 | 100.0 | 103.815 | 103,800 |

¹The volume of the dose is 100 μL.

TABLE 26
Characteristics of exemplary formulation.

| Physical Test | Results |
|---|---|
| pH | 4.1 |
| Osmolality w/Glycerin (mOsm) | 615 |
| Density δ (g/mL) | 1.038 |
| Osmolality range | 400-850 |
| (mOsm)(preliminary) | |

The formulation of Example 10 can be prepared and characterized based on the procedure described in Table 27.

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A pharmaceutical composition comprising naloxone hydrochloride or a hydrate thereof and glycerin.

2. The pharmaceutical composition of claim 1, wherein the pharmaceutical composition is an aqueous solution.

3. The pharmaceutical composition of claim 1 or 2, wherein the pharmaceutical composition comprises propylene glycol.

4. The pharmaceutical composition of any one of claims 1 to 3, wherein the pharmaceutical composition comprises chlorobutanol.

5. The pharmaceutical composition of any one of claims 1 to 4, wherein the pharmaceutical composition comprises an acid.

6. The pharmaceutical composition of claim 5, wherein the acid is citric acid.

7. The pharmaceutical composition of any one of claims 1 to 6, wherein the pharmaceutical composition comprises a buffer.

8. The pharmaceutical composition of claim 7, wherein the buffer comprises citric acid and trisodium citrate dihydrate.

9. The pharmaceutical composition of claim 8, wherein the buffer is an acetate buffer.

10. The pharmaceutical composition of any one of claims 1 to 9, wherein the pharmaceutical composition comprises an isotonicity agent.

11. The pharmaceutical composition of claim 10, wherein the isotonicity agent is sodium chloride.

12. The pharmaceutical composition of any one of claims 1 to 11, wherein the composition comprises a stabilizing agent.

13. The pharmaceutical composition of claim 12, wherein the stabilizing agent is selected from the group consisting of EDTA and disodium ETDA.

14. The pharmaceutical composition of any one of claims 2 to 13, wherein the volume of the aqueous solution is from about 50 μL to about 200 μL.

15. The pharmaceutical composition of any one of claims 2 to 14, wherein the volume of the aqueous solution is from about 80 μL to about 150 μL.

16. The pharmaceutical composition of any one of claims 2 to 15, wherein the volume of the aqueous solution is from about 90 μL to about 120 μL.

17. The pharmaceutical composition of any one of claims 2 to 16, wherein the volume of the aqueous solution is about 100 μL of the aqueous solution.

18. The pharmaceutical composition of any one of claims 2 to 17, wherein the volume of the aqueous solution is 133 μL of the aqueous solution.

19. The pharmaceutical composition of any one of claims 2 to 18, wherein the concentration of naloxone hydrochloride or hydrate thereof is from about 2 mg per 100 μL to about 10 mg per 100 μL of the aqueous solution.

20. The pharmaceutical composition of any one of claims 2 to 19, wherein the concentration of naloxone hydrochloride or hydrate thereof is from about 4 mg per 100 μL to about 8 mg per 100 μL of the aqueous solution.

21. The pharmaceutical composition of any one of claims 2 to 20, wherein the concentration of naloxone hydrochloride or hydrate thereof is selected from the group consisting of about 4 mg per 100 μL, about 4.4 mg per 100 μL, about 6 mg per 100 μL, and about 8 mg per 133 μL of the aqueous solution of the aqueous solution.

22. The pharmaceutical composition of any one of claims 2 to 21, wherein the concentration of naloxone hydrochloride or hydrate thereof is about 4 mg per 100 μL of the aqueous solution.

23. The pharmaceutical composition of any one of claims 2 to 21, wherein the concentration of naloxone hydrochloride or hydrate thereof is about 4.4 mg per 100 μL of the aqueous solution.

24. The pharmaceutical composition of any one of claims 2 to 21, wherein the concentration of naloxone hydrochloride or hydrate thereof is about 6 mg per 100 μL of the aqueous solution.

25. The pharmaceutical composition of any one of claims 2 to 21, wherein the concentration of naloxone hydrochloride or hydrate thereof is about 8 mg per 133 μL of the aqueous solution.

26. The pharmaceutical composition of any one of claims 1 to 25, wherein the hydrate of naloxone hydrochloride is naloxone hydrochloride dihydrate.

27. The pharmaceutical composition of any one of claims 2 to 26, wherein the concentration of chlorobutanol is from about 0.1 mg per 100 μL to about 0.8 mg per 100 μL of the aqueous solution.

28. The pharmaceutical composition of any one of claims 2 to 27, wherein the concentration of chlorobutanol is from about 0.2 mg per 100 μL to about 0.6 mg per 100 μL of the aqueous solution.

29. The pharmaceutical composition of any one of claims 2 to 28, wherein the concentration of chlorobutanol is from about 0.3 mg per 100 μL to about 0.5 mg per 100 μL of the aqueous solution.

30. The pharmaceutical composition of any one of claims 2 to 29, wherein the concentration of chlorobutanol is from about 0.4 mg per 100 μL to about 0.5 mg per 100 μL of the aqueous solution.

31. The pharmaceutical composition of any one of claims 2 to 30, wherein the concentration of chlorobutanol is about 0.4 mg per 100 μL of the aqueous solution.

32. The pharmaceutical composition of any one of claims 2 to 31, wherein the concentration of chlorobutanol is about 0.45 mg per 100 μL of the aqueous solution.

33. The pharmaceutical composition of any one of claims 2 to 32, wherein the concentration of chlorobutanol is about 0.53 mg per 100 μL of the aqueous solution.

34. The pharmaceutical composition of any one of claims 2 to 33, wherein the concentration of the acid is from about 0.001 mg per 100 μL to about 0.01 mg per 100 μL of the aqueous solution.

35. The pharmaceutical composition of any one of claims 2 to 34, wherein the concentration of the acid is no more than about 0.15 mg per 100 μL of the aqueous solution.

36. The pharmaceutical composition of any one of claims 2 to 35, wherein the concentration of the acid is from about 0.001 mg per 100 μL to about 0.15 mg per 100 μL of the aqueous solution.

37. The pharmaceutical composition of any one of claims 2 to 36, wherein the concentration of the acid is from about 0.002 mg per 100 μL to about 0.009 mg per 100 μL of the aqueous solution.

38. The pharmaceutical composition of any one of claims 2 to 37, wherein the concentration of the acid is from about 0.004 mg per 100 μL to about 0.009 mg per 100 μL of the aqueous solution.

39. The pharmaceutical composition of any one of claims 2 to 38, wherein the concentration of the acid is from about 0.005 mg per 100 μL to about 0.009 mg per 100 μL of the aqueous solution.

40. The pharmaceutical composition of any one of claims 2 to 39, wherein the concentration of the acid is from about 0.006 mg per 100 μL to about 0.009 mg per 100 μL of the aqueous solution.

41. The pharmaceutical composition of any one of claims 2 to 40, wherein the concentration of the acid is from about 0.006 mg per 100 μL to about 0.008 mg per 100 μL of the aqueous solution.

42. The pharmaceutical composition of any one of claims 2 to 41, wherein the concentration of the acid is about 0.009 mg per 100 μL of the aqueous solution.

43. The pharmaceutical composition of any one of claims 2 to 42, wherein the concentration of the buffer is no more than about 0.15 mg per 100 μL of the aqueous solution.

44. The pharmaceutical composition of any one of claims 2 to 43, wherein the concentration of the buffer is from about 0.001 mg per 100 μL to about 0.15 mg per 100 μL of the aqueous solution.

45. The pharmaceutical composition of any one of claims 2 to 44, wherein the concentration of the buffer is from about 0.002 mg per 100 μL to about 0.02 mg per 100 μL of the aqueous solution.

46. The pharmaceutical composition of any one of claims 2 to 45, wherein the concentration of the buffer is from about 0.004 mg per 100 μL to about 0.015 mg per 100 μL of the aqueous solution.

47. The pharmaceutical composition of any one of claims 2 to 46, wherein the concentration of the buffer is from about 0.008 mg per 100 μL to about 0.012 mg per 100 μL of the aqueous solution.

48. The pharmaceutical composition of any one of claims 2 to 47, wherein the concentration of the buffer is from about 0.009 mg per 100 μL to about 0.011 mg per 100 μL of the aqueous solution.

49. The pharmaceutical composition of any one of claims 2 to 48, wherein the concentration of the buffer is about 0.01 mg per 100 μL of the aqueous solution.

50. The pharmaceutical composition of any one of claims 2 to 49, wherein the concentration of the buffer is about 0.019 mg per 100 μL of the aqueous solution.

51. The pharmaceutical composition of any one of claims 2 to 50, wherein the concentration of the buffer is about 0.02 mg per 100 μL of the aqueous solution.

52. The pharmaceutical composition of any one of claims 2 to 51, wherein the concentration of the isotonicity agent is no more than about 2 mg per 100 μL of the aqueous solution.

53. The pharmaceutical composition of any one of claims 2 to 52, wherein the concentration of the isotonicity agent is from about 0.1 mg per 100 μL to about 2 mg per 100 μL of the aqueous solution.

54. The pharmaceutical composition of any one of claims 2 to 53, wherein the concentration of the isotonicity agent is from about 0.4 mg per 100 μL to about 1.5 mg per 100 μL of the aqueous solution.

55. The pharmaceutical composition of any one of claims 2 to 54, wherein the concentration of the isotonicity agent is from about 0.5 mg per 100 μL to about 1 mg per 100 μL of the aqueous solution.

56. The pharmaceutical composition of any one of claims 2 to 55, wherein the concentration of the isotonicity agent is from about 0.7 mg per 100 μL to about 0.9 mg per 100 μL of the aqueous solution.

57. The pharmaceutical composition of any one of claims 2 to 56, wherein the concentration of the isotonicity agent is from about 0.8 mg per 100 μL to about 0.9 mg per 100 μL of the aqueous solution.

58. The pharmaceutical composition of any one of claims 2 to 57, wherein the concentration of the isotonicity agent is about 0.85 mg per 100 μL of the aqueous solution.

59. The pharmaceutical composition of any one of claims 2 to 58, wherein the concentration of the isotonicity agent is about 0.625 mg per 100 μL of the aqueous solution.

60. The pharmaceutical composition of any one of claims 2 to 59, wherein the concentration of the glycerin is from about 0.1 mg per 100 μL to about 2 mg per 100 μL of the aqueous solution.

61. The pharmaceutical composition of any one of claims 2 to 60, wherein the concentration of the glycerin is from about 0.4 mg per 100 μL to about 1.8 mg per 100 μL of the aqueous solution.

62. The pharmaceutical composition of any one of claims 2 to 61, wherein the concentration of the glycerin is from about 0.8 mg per 100 μL to about 1.6 mg per 100 μL of the aqueous solution.

63. The pharmaceutical composition of any one of claims 2 to 62, wherein the concentration of the glycerin is from about 1 mg per 100 μL to about 1.6 mg per 100 μL of the aqueous solution.

64. The pharmaceutical composition of any one of claims 2 to 63, wherein the concentration of the glycerin is from about 1.3 mg per 100 μL to about 1.5 mg per 100 μL of the aqueous solution.

65. The pharmaceutical composition of any one of claims 2 to 64, wherein the concentration of the glycerin is about 1.4 mg per 100 μL of the aqueous solution.

66. The pharmaceutical composition of any one of claims 2 to 65, wherein the osmolality of the composition is from about 300 mOsm to about 700 mOsm.

67. The pharmaceutical composition of any one of claims 2 to 66, wherein the osmolality of the composition is from about 400 mOsm to about 700 mOsm.

68. The pharmaceutical composition of any one of claims 2 to 67, wherein the osmolality of the composition is from about 400 mOsm to about 650 mOsm.

69. The pharmaceutical composition of any one of claims 2 to 68, wherein the osmolality of the composition is about 446 mOsm.

70. The pharmaceutical composition of any one of claims 2 to 69, wherein the osmolality of the composition is about 614 mOsm.

71. The pharmaceutical composition of any one of claims 2 to 70, wherein the osmolality of the composition is about 607 mOsm.

72. The pharmaceutical composition of any one of claims 2 to 71, wherein the osmolality of the composition is about 446 mOsm and the composition does not comprise glycerin.

73. The pharmaceutical composition of any one of claims 2 to 72, wherein the osmolality of the composition is about 614 mOsm and the composition comprises glycerin.

74. The pharmaceutical composition of any one of claims 2 to 73, wherein the osmolality of the composition is about 607 mOsm and the composition comprises glycerin.

75. The pharmaceutical composition of any one of claims 2 to 74, wherein the osmolality of the composition is about 614 mOsm, the concentration of naloxone hydrochloride dihydrate is about 4 mg per 100 μL of aqueous solution, and the composition comprises glycerin.

76. The pharmaceutical composition of any one of claims 2 to 75, wherein the osmolality of the composition is about 607 mOsm, the concentration of naloxone hydrochloride dihydrate is about 6 mg per 100 μL of aqueous solution, and the composition comprises glycerin.

77. The pharmaceutical composition of any one of claims 2 to 76, wherein the osmolality of the composition is about 607 mOsm, the concentration of naloxone hydrochloride dihydrate is about 8 mg per 133 μL of aqueous solution, and the composition comprises glycerin.

78. The pharmaceutical composition of any one of claims 1 to 77, wherein the pH of the composition is from about 3 to about 6.

79. The pharmaceutical composition of any one of claims 1 to 78, wherein the pH of the composition is from about 4 to about 5.

80. The pharmaceutical composition of any one of claims 1 to 79, wherein the pH of the composition is about 4.1.

81. The pharmaceutical composition of any one of claims 1 to 80, wherein the pH of the composition is about 4.0.

82. The pharmaceutical composition of any one of claims 1 to 81, wherein the composition is formulated for intranasal administration.

83. The pharmaceutical composition of any one of claims 1 to 82, wherein, when intranasally administered to a subject, the pH of the composition is the pH of the nasal mucosa of the subject.

84. The pharmaceutical composition of any one of claims 1 to 83, which yields, when intranasally administered to a subject, a naloxone Tₘₐₓ of less than 30 minutes.

85. The pharmaceutical composition of any one of claims 1 to 84, which yields, when intranasally administered to a subject, a naloxone Tₘₐₓ of less than 25 minutes.

86. The pharmaceutical composition of any one of claims 1 to 85, which yields, when intranasally administered to a subject, a naloxone Tₘₐₓ of less than 20 minutes.

87. The pharmaceutical composition of any one of claims 1 to 86, which yields, when intranasally administered to a subject, a mean naloxone plasma concentration of ≥0.2 ng/mL within 2.5 minutes in said subject.

88. The pharmaceutical composition of any one of claims 1 to 87, which yields, when intranasally administered to a subject, a mean naloxone plasma concentration of ≥1 ng/mL within 5 minutes in said subject.

89. The pharmaceutical composition of any one of claims 1 to 88, which yields, when intranasally administered to a subject, a mean naloxone plasma concentration of ≥3 ng/mL within 10 minutes in said subject.

90. A pharmaceutical composition comprising naloxone hydrochloride or a hydrate thereof, chlorobutanol, citric acid, trisodium citrate dihydrate, sodium chloride, and glycerin.

91. The pharmaceutical composition of claim 90, wherein the hydrate of naloxone hydrochloride is naloxone hydrochloride dihydrate.

92. The pharmaceutical composition of claim 90, wherein the concentration of naloxone hydrochloride dihydrate is selected from the group consisting of about 4 mg per 100 μL of composition, about 6 mg per 100 μL of composition, and about 8 mg per 133 μL of composition the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is selected from the group consisting of about 0.85 mg per 100 μL of composition and about 0.625 mg per 100 μL of composition, and the concentration of glycerin is about 1.4 mg per 100 μL of composition.

93. The pharmaceutical composition of claim 90, wherein the concentration of naloxone hydrochloride dihydrate is about 4 mg per 100 μL of composition, the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is about 0.85 mg per 100 μL of composition, and the concentration of glycerin is about 1.4 mg per 100 μL of composition.

94. The pharmaceutical composition of claim 90, wherein the concentration of naloxone hydrochloride dihydrate is selected from the group consisting of about 6 mg per 100 μL of composition, the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is selected from the group consisting of about 0.625 mg per 100 μL of composition, and the concentration of glycerin is about 1.4 mg per 100 μL of composition.

95. The pharmaceutical composition of claim 90, wherein the concentration of naloxone hydrochloride dihydrate is selected from the group consisting of about 8 mg per 133 μL of composition the concentration of chlorobutanol is about 0.45 mg per 100 μL of composition, the concentration of citric acid is about 0.009 mg per 100 μL of composition, the concentration of trisodium citrate dihydrate is about 0.01 mg per 100 μL of composition, the concentration of sodium chloride is selected from the group consisting of about 0.625 mg per 100 μL of composition, and the concentration of glycerin is about 1.4 mg per 100 μL of composition.

96. The pharmaceutical composition of any one of claims 90 to 95, wherein the pharmaceutical composition is an aqueous solution.

97. The pharmaceutical composition of any one of claims 90 to 96, wherein the volume of the aqueous solution is from about 50 μL to about 200 μL.

98. The pharmaceutical composition of any one of claims 90 to 97, wherein the volume of the aqueous solution is from about 80 μL to about 150 μL.

99. The pharmaceutical composition of any one of claims 90 to 98, wherein the volume of the aqueous solution is from about 90 μL to about 120 μL.

100. The pharmaceutical composition of any one of claims 90 to 99, wherein the volume of the aqueous solution is about 100 μL of the aqueous solution.

101. The pharmaceutical composition of any one of claims 90 to 100, wherein the volume of the aqueous solution is about 133 μL of the aqueous solution.

102. A method of treating an opioid overdose or symptom thereof in a subject in need thereof, comprising administering to the subject a pharmaceutical composition according to any one of claims 1 to 101.

103. The method of claim 102, wherein the pharmaceutical composition comprises a therapeutically effective amount of naloxone hydrochloride or a hydrate thereof.

104. The method of claim 102 or claim 103, wherein the hydrate of naloxone hydrochloride is naloxone hydrochloride dihydrate.

105. The method of claim 103 or claim 104, wherein the therapeutically effective amount is from about 2 mg to about 10 mg of the aqueous solution.

106. The method of any one of claims 102 to 105, wherein the therapeutically effective amount is from about 4 mg to about 8 mg.

107. The method of any one of claims 102 to 106, wherein the therapeutically effective amount is selected from the group consisting of about 4 mg, about 6 mg, and about 8 mg.

108. The method of any one of claims 102 to 107, wherein the therapeutically effective amount is about 4 mg.

109. The method of any one of claims 102 to 108, wherein the therapeutically effective amount is about 6 mg.

110. The method of any one of claims 102 to 109, wherein the therapeutically effective amount is about 8 mg.

111. The method of any one of claims 102 to 110, wherein upon nasal delivery of said pharmaceutical composition to said subject, less than about 20% of said pharmaceutical composition leaves the nasal cavity via drainage into the nasopharynx or externally.

112. The method of any one of claims 102 to 111, wherein upon nasal delivery of said pharmaceutical composition to said subject, less than about 10% of said pharmaceutical composition leaves the nasal cavity via drainage into the nasopharynx or externally.

113. The method of any one of claims 102 to 112, wherein upon nasal delivery of said pharmaceutical composition to said subject, less than about 5% of said pharmaceutical composition leaves the nasal cavity via drainage into the nasopharynx or externally.

114. The method of any one of claims 102 to 113, wherein the plasma concentration versus time curve of said naloxone hydrochloride or hydrate thereof in said subject has a Tₘₐₓ of between about 20 and about 30 minutes.

115. The method of any one of claims 102 to 114, wherein the administration yields a mean naloxone plasma concentration of ≥0.2 ng/mL within 2.5 minutes in said subject.

116. The method of any one of claims 102 to 115, wherein the administration yields a mean naloxone plasma concentration of ≥1 ng/mL within 5 minutes in said subject.

117. The method of any one of claims 102 to 116, wherein the administration yields a mean naloxone plasma concentration of ≥3 ng/mL within 10 minutes in said subject.

118. The method of any one of claims 102 to 117, wherein the subject exhibits one or more symptoms selected from the group consisting of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting.

119. The method of any one of claims 102 to 118, wherein the subject exhibits respiratory depression.

120. The method of any one of claims 102 to 119, wherein the opioid overdose or symptom thereof is caused by an opioid selected from the group consisting of codeine, morphine, methadone, fentanyl, carfentanyl, acetyl fentanyl, oxycodone hydrochloride, hydrocodone bitartrate, hydromorphone, oxymorphone, meperidine, propoxyphene, opium, heroin, tramadol, tapentadol, and narcotic-antagonist analgesics.

121. The method of claim 120, wherein the narcotic-antagonist analgesics is selected from the group consisting of nalbuphine, pentazocine, and butorphanol.

122. The method of any one of claims 102 to 121, wherein the subject is a mammal.

123. The method of any one of claims 102 to 122, wherein the subject is a human.

124. The method of any one of claims 102 to 123, wherein the subject is an opioid overdose subject or a suspected opioid overdose subject.

125. A device configured for intranasally administering of a pharmaceutical composition of any one of claims 1 to 101 to a subject, wherein the device is configured for delivery of one dose of the pharmaceutical composition to the subject.

126. The device of claim 125, wherein the dose is contained in a single reservoir.

127. The device of claim 125 or claim 126, wherein the device is adapted for single use.

128. The device of any one of claims 125 to 127, wherein the device is configured for delivery of the dose to the subject by a single actuation.

129. The device of any one of claims 125 to 128, wherein the device is not primed prior administering the dose to the subject.

130. The device of any one of claims 125 to 129, wherein the volume of said reservoir is not more than about 200 μL.

131. The device of any one of claims 125 to 130, wherein the volume of said reservoir is not more than about 140 μL.

132. The device of any one of claims 125 to 131, wherein the volume of the single dose in the reservoir is from about 90 μL to about 140 μL.

133. The device of any one of claims 125 to 132, wherein the volume of the single dose in the reservoir is from about 95 μL to about 135 μL.

134. The device of any one of claims 125 to 133, wherein the volume of the single dose in the reservoir is about 100 μL.

135. The device of any one of claims 125 to 134, wherein the volume of the single dose in the reservoir is about 133 μL.

136. The device of any one of claims 125 to 135, wherein the device is configured to deliver the single dose at a volume from about 90 μL to about 140 μL.

137. The device of any one of claims 125 to 136, wherein the device is configured to deliver the single dose at a volume from about 95 μL to about 135 μL.

138. The device of any one of claims 125 to 137, wherein the device is configured to deliver the single dose at a volume of about 100 μL.

139. The device of any one of claims 125 to 138, wherein the device is configured to deliver the single dose at a volume of about 133 μL.

140. The device of any one of claims 125 to 139, wherein the device is actuatable with one hand.

141. The device of any one of claims 125 to 140, wherein the device is configured such that the 90% confidence interval for dose delivered per actuation is ±about 2%.

142. The device of any one of claims 125 to 141, wherein the device is configured such that the 95% confidence interval for dose delivered per actuation is ±about 2.5%.

143. The device of any one of claims 125 to 142, wherein the device is configured such that the delivery time is less than about 25 seconds.

144. The device of any one of claims 125 to 143, wherein the device is configured such that the delivery time is less than about 20 seconds.

145. The device of any one of claims 125 to 144, wherein upon nasal delivery of said pharmaceutical composition to said subject, less than about 20% of said pharmaceutical composition leaves the nasal cavity via drainage into the nasopharynx or externally.

146. The device of any one of claims 125 to 145, wherein upon nasal delivery of said pharmaceutical composition to said subject, less than about 10% of said pharmaceutical composition leaves the nasal cavity via drainage into the nasopharynx or externally.

147. The device of any one of claims 125 to 146, wherein upon nasal delivery of said pharmaceutical composition to said subject, less than about 5% of said pharmaceutical composition leaves the nasal cavity via drainage into the nasopharynx or externally.

148. The device of any one of claims 125 to 147, wherein the plasma concentration versus time curve of the naloxone hydrochloride or hydrate thereof in said subject has a Tₘₐₓ of between about 10 and about 30 minutes.

149. The device of any one of claims 125 to 148, wherein the subject exhibits one or more symptoms selected from the group consisting of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing; erratic or stopped pulse, choking or gurgling sounds, blue or purple fingernails or lips, slack or limp muscle tone, contracted pupils, and vomiting.

150. The device of any one of claims 125 to 149, wherein the subject exhibits respiratory depression.

151. The device of any one of claims 125 to 150, wherein said respiratory depression is caused by the illicit use of opioids, or by an accidental misuse of opioids during medical opioid therapy.

152. The device of any one of claims 125 to 151, wherein said subject is free from respiratory depression for at least about 1 hour following treatment comprising essentially of delivery of said therapeutically effective amount of said opioid antagonist.

153. The device of any one of claims 125 to 152, wherein said subject is free from respiratory depression for at least about 2 hours following treatment comprising essentially of delivery of said therapeutically effective amount of said opioid antagonist.

154. The device of any one of claims 125 to 153, wherein said subject is free from respiratory depression for at least about 4 hours following treatment comprising essentially of delivery of said therapeutically effective amount of said opioid antagonist.

155. The device of any one of claims 125 to 154, wherein said subject is free from respiratory depression for at least about 6 hours following treatment comprising essentially of delivery of said therapeutically effective amount of said opioid antagonist.

156. The device of any one of claims 125 to 155, wherein said subject is in a lying, supine, or recovery position.

157. The device of any one of claims 125 to 156, wherein said single actuation yields a plasma concentration of ≥0.2 ng/mL within 2.5 minutes in said subject.

158. The device of any one of claims 125 to 157, wherein said single actuation yields a plasma concentration of ≥1 ng/mL within 5 minutes in said subject.

159. The device of any one of claims 125 to 158, wherein said single actuation yields a plasma concentration of ≥3 ng/mL within 10 minutes in said subject.

160. The device of any one of claims 125 to 159, wherein said single actuation yields a plasma concentration of ≥0.2 ng/mL within 2.5 minutes in said subject.

161. The device of any one of claims 125 to 160, wherein said single actuation yields a plasma concentration of ≥1 ng/mL within 5 minutes in said subject.

162. The device of any one of claims 125 to 161, wherein the subject is a mammal.

163. The device of any one of claims 125 to 162, wherein the subject is a human.

164. The device of any one of claims 125 to 163, wherein said subject is an opioid overdose subject or a suspected opioid overdose subject.

165. The device of any one of claims 125 to 164, wherein the pharmaceutical composition is administered as a spray of droplets to the subject.

166. The device of claim 165, wherein the Dv90 of the spray of droplets is from about 48 μm to about 80 μm.

167. The device of claim 165 or claim 166, wherein the Dv90 of the spray of droplets is from about 60 μm to about 80 μm.

168. The device of any one of claims 165 to 167, wherein the Dv90 of the droplets is from about 56.77 μm.

169. The device of any one of claims 165 to 168, wherein the Dv90 of the droplets is from about 55.10 μm.

170. The device of claim 165, wherein the Dv50 of the droplets is from about 20 μm to about m.

171. The device of claim 165 or claim 170, wherein the Dv50 of the droplets is from about 25 μm to about 40 μm.

172. The device of claim 165 or claim 170, wherein the Dv50 of the droplets is from about 22 μm to about 34 μm.

173. The device of claim 165 or claim 170, wherein the Dv50 of the droplets is about 24.72 μm.

174. The device of claim 165 or claim 170, wherein the Dv50 of the droplets is about 24.75 μm.

175. The device of claim 165, wherein the Dv10 of the droplets is from about 10 μm to about m.

176. The device of claim 165 or claim 175, wherein the Dv10 of the droplets is from about 12 μm to about 20 μm.

177. The device of claim 165 or claim 175, wherein the Dv10 of the droplets is from about 10 μm to about 17 μm.

178. The device of claim 165 or claim 175, wherein the Dv10 of the droplets is about 11.35 μm.

179. The device of claim 165 or claim 175, wherein the Dv10 of the droplets is about 11.47 μm.

180. The device of claim 165, claim 178, or claim 179, wherein the percent volume of droplets less than 10 μm is less than about 12%.

181. The device of claim 165, claim 178, or claim 179, wherein the percent volume of droplets less than 10 μm is less than about 10%.

182. The device of claim 165, claim 178, or claim 179, wherein the percent volume of droplets less than 10 μm is about 6.7%.

183. The device of claim 165, claim 178, or claim 179, wherein the percent volume of droplets less than 10 μm is about 6.3%.

184. The device of claim 165, wherein the device sprays a spray pattern with a Dmax of about 50 mm.

185. The device of claim 165, wherein the device sprays a spray pattern with a Dmax of about 40.2 mm.

186. The device of claim 165, wherein the device sprays a spray pattern with a Dmax of about 40.7 mm.

187. The device of claim 165, wherein the device sprays a spray pattern with an area of about 750 mm² to about 1500 mm².

188. The device of claim 165, wherein the device sprays a spray pattern with an area of about 1100 mm².

189. The device of claim 165, wherein the device sprays a spray pattern with an area of about 1110 mm².

190. The device of claim 165, wherein the device sprays a spray pattern with an ovality ratio of about 1.0 to about 2.5.

191. The device of claim 165, wherein the device sprays a spray pattern with an ovality ratio of about 1.2.

192. A method of treating an opioid overdose or a symptom thereof, comprising intranasally administering to the subject a pharmaceutical composition comprising greater than 4 mg of naloxone hydrochloride or a hydrate thereof and glycerin.

193. The method of claim 192, wherein the pharmaceutical composition is an aqueous solution.

194. The method of claim 192 or claim 193, wherein the pharmaceutical composition is administered to the subject in a single dose.

195. The method of any one of claims 192 to 194, wherein the concentration of naloxone hydrochloride is about 6 mg per 100 μL of aqueous solution.

196. The method of any one of claims 192 to 195, wherein the total mass of naloxone hydrochloride administered is not more than about 20 mg.

197. The method of any one of claims 192 to 196, wherein the total mass of the hydrate of naloxone hydrochloride administered is not more than about 20 mg.

198. The method of any one of claims 192 to 197, wherein the total administered volume is from about 30 μL to about 200 μL.

199. The method of any one of claims 192 to 198, wherein the total administered volume is from about 50 μL to about 150 μL.

200. The pharmaceutical composition of any one of claims 2 to 101, wherein the composition is an aqueous solution and the concentration of naloxone hydrochloride or hydrate thereof is about 10 mg per 100 μL of aqueous solution to about 16 mg per 100 μL of aqueous solution.

201. The pharmaceutical composition of claims 2 to 101, wherein the composition is an aqueous solution and the concentration of naloxone hydrochloride or hydrate thereof is about 12 mg per 100 μL of aqueous solution and the concentration of glycerin is about 1.4 mg 100 μL of aqueous solution.

202. The pharmaceutical composition of claims 2 to 101, wherein the composition is an aqueous solution and the concentration of naloxone hydrochloride or hydrate thereof is about 12 mg per 100 μL of aqueous solution and the concentration of glycerin is about 2.5 mg 100 μL of aqueous solution.

203. A pharmaceutical composition comprising naloxone hydrochloride or hydrate thereof and a polyol less than 300 Da.

204. The pharmaceutical composition of claim 203, wherein the polyol less than 300 Da is glycerin.

205. The pharmaceutical composition of claim 203, wherein the polyol less than 300 Da is propylene glycol.
